(19) United States
(12) Patent Application Publication
(10) Pub. No.: US 2015/0208933 A1
(43) Pub. Date: Jul. 30, 2015

(54) PULSE WAVE SENSOR

(71) Applicant: ROHM CO., LTD., Kyoto (JP)

(72) Inventors: Tsuyoshi Satomi, Kyoto (JP); Hiroki Miyabuchi, Kyoto (JP); Koji Terumoto, Kyoto (JP)

(21) Appl. No.: 14/427,725

(22) PCT Filed: Sep. 13, 2013
(86) PCT No.: PCT/JP2013/074865

§ 371 Date: Mar. 12, 2015

(30) Foreign Application Priority Data
Sep. 28, 2012 (JP) 2012-215864

(51) Int. Cl. A61B 5/024; A61B 5/00

(52) U.S. Cl. CPC A61B 5/02427; A61B 5/6815

(57) ABSTRACT

[Problem] To provide a pulse sensor capable of accurately measuring the pulse of a subject. [Solution] A pulse sensor (600) has: a housing (610) mounted to an external ear; an optical sensor unit (620) which is disposed upon the housing (610) and acquires pulse data by emitting light from a light-emitting portion onto the external ear and detecting, at a light-receiving unit, the intensity of the light that is transmitted through a living body and returns; and a buffer member (630) which is disposed between the housing (610) and the optical sensor unit (620).

### TECHNICAL FIELD

The present invention relates to pulse wave sensors.

### BACKGROUND ART

Conventionally, a pulse wave sensor achieves pulse wave measurement by use of a light emitter which irradiates a test subject's finger tip or the like with infrared light and a light receiver which detects the intensity of the infrared light that has passed through the living body.

Examples of the conventional technology mentioned above are seen in Patent Documents 1 and 2 identified below.

On the other hand, there have conventionally been proposed technologies for detecting the condition of sleep based on a test subject's pulse waves (see, for example, Patent Document 3).

### LIST OF CITATIONS

### Patent Literature

### Patent Document 1: JP-A-H5-212016

### Patent Document 2: WO 2002/062222

### Patent Document 3: JP-A-2003-79588

### SUMMARY OF THE INVENTION

### Technical Problem

However, conventional pulse wave sensors are basically designed to measure pulse waves while the test subject is at rest, and with them it is difficult to measure pulse waves with high accuracy while the test subject is in activity.

Moreover, with the conventional structure, which achieves pulse wave measurement at a finger tip, the test subject's activities need to be restricted so that the pulse wave sensor will not drop off the finger tip during pulse wave measurement. Moreover, pulse wave measurement at a finger tip also has the disadvantage of being prone to be affected by noise resulting from the test subject's motion.

Moreover, conventional pulse wave sensors are basically designed to measure pulse waves indoors, and with them it is difficult to measure pulse waves with high accuracy outdoors.

On the other hand, conventional sleep sensors are designed to detect the condition of the test subject's sleep based on a single source of living body information (such as pulse waves), and their detection accuracy leaves room for further improvement. Moreover, conventional sleep sensors are designed to operate on their own, and are not supposed to be used to build a physical condition management system or a home appliance control system.

It is an object of one of different aspects of the present invention disclosed herein to provide a pulse wave sensor that allows accurate measurement of a test subject's pulse waves.

### Means for Solving the Problem

According to one aspect disclosed herein, a pulse wave sensor has a housing which is worn on the outer ear; an optical sensor which is provided in the housing and which acquires pulse wave data by irradiating the outer ear with light from a light emitter and detecting with a light receiver the intensity of the light returning after passing through the living body; and a damping member which is provided between the housing and the optical sensor (Configuration 1).

The pulse wave sensor of Configuration 1 can be so configured as to further have a close-contact member which enhances the ease of wearing on the outer ear (Configuration 2).

The pulse wave sensor of Configuration 2 can be so configured that the optical sensor is arranged at a position where the optical sensor is covered by the close-contact member, which transmits light (Configuration 3).

The pulse wave sensor of Configuration 3 can be so configured that the damping member is arranged between the housing and the optical sensor with the damping member compressed in its height direction (Configuration 4).

The pulse wave sensor of Configuration 4 can be so configured that the damping member is compressed by the contracting force of the close-contact member which covers the optical sensor (Configuration 5).

The pulse wave sensor of Configuration 4 or 5 can be so configured that the damping member is compressed by the binding force of leads which are laid from opposite ends of the optical sensor (Configuration 6).

The pulse wave sensor of any of Configurations 4 to 6 can be so configured that the damping member is compressed by the contracting force of an elastic member which couples the housing and the optical sensor together (Configuration 7).

The pulse wave sensor of any of Configurations 4 to 7 can be so configured that the damping member is compressed by the locking force of a protruding member which couples the housing and the optical sensor together.

The pulse wave sensor of any of Configurations 4 to 8 can be so configured that the damping member, when uncompressed, has a height of 2.5±1.0 cm (Configuration 9).

The pulse wave sensor of any of Configurations 4 to 9 can be so configured as to further have a light-shielding member which prevents outside light from entering the optical sensor (Configuration 10).

The pulse wave sensor of Configuration 10 can be so configured that the close-contact member transmits light at the light emission wavelength only in a part of the close-contact member covering the optical sensor to serve as a measurement window, and elsewhere functions as the light-shielding member (Configuration 11).

The pulse wave sensor of any of Configurations 1 to 11 can be so configured that the damping member is formed of urethane sponge (Configuration 12).

The pulse wave sensor of any of Configurations 1 to 12 can be so configured that the light receiver is arranged closer to the external ear canal than the light emitter is (Configuration 13).

The pulse wave sensor of any of Configurations 1 to 13 can be so configured that the output wavelength of the light emitter is in a visible region of the spectrum, about 600 nm or less (Configuration 14).

According to another aspect disclosed herein, a pulse wave sensor has a housing which is worn on the outer ear; an optical sensor which is provided in the housing and which acquires pulse wave data by irradiating the outer ear with light from a light emitter and detecting with a light receiver the intensity of the light returning after passing through the living body; and a close-contact member which enhances the closeness of contact between the optical sensor and the outer ear (Configuration 15).

According to yet another aspect disclosed herein, a pulse wave sensor has a housing which is worn on the outer ear; an optical sensor which is provided in the housing and which acquires pulse wave data by irradiating the outer ear with light from a light emitter and detecting with a light receiver the intensity of the light returning after passing through the living body; and a light-shielding member which prevents outside light from entering the optical sensor (Configuration 16).

### Advantageous Effects of the Invention

With a pulse wave sensor disclosed herein, it is possible to measure a test subject's pulse waves with high accuracy irrespective of the test subject's motion (at rest or in activity), and irrespective of the place of pulse wave measurement (indoors or outdoors). This helps widen the scope of use of a pulse wave sensor.

### BRIEF DESCRIPTION OF DRAWINGS

FIG. 1 is a schematic diagram illustrating the principle of pulse wave measurement on the wrist;

FIG. 2 is a waveform chart showing how the level of light attenuation (the degree of light absorption) through the living body varies with time;

FIG. 3 is a block diagram of an optical sensor 11 according to a first embodiment of the present invention;

FIG. 4 is a sectional view showing a first configuration example of the optical sensor 11;

FIG. 5 is a sectional view showing a second configuration example of the optical sensor 11;

FIG. 6 is a waveform chart showing the correlation between offset distance ΔH and signal strength;

FIG. 7 is a waveform chart showing the correlation between chip-to-chip distance W1 and signal strength;

FIG. 8A is a sectional view showing a third configuration example of the optical sensor 11;

FIG. 8B is a sectional view showing a fourth configuration example of the optical sensor 11;

FIG. 8C is a sectional view showing a fifth configuration example of the optical sensor 11;

FIG. 8D is a sectional view showing a sixth configuration example of the optical sensor 11;

FIG. 9 is a sectional view showing a seventh configuration example of the optical sensor 11;

FIG. 10 is a layout diagram showing an arrangement of the optical sensor 11 in a wrist watch-type pulse wave sensor 1;

FIG. 11 is a waveform chart showing the correlation between arrangement of the optical sensor 11 and signal strength;

FIG. 12 is a layout diagram showing an arrangement of the optical sensor 11 in an earring-type pulse wave sensor 1;

FIG. 13 is a circuit diagram showing a first configuration example of the filter 12;

FIG. 14 is a circuit diagram showing a second configuration example of the filter 12;

FIG. 15 is an output waveform chart of the filter 12;

FIG. 16 is a block diagram showing a pulse wave sensor according to a second embodiment of the present invention;

FIG. 17 is a sectional view schematically showing the mechanism by which body motion noise is produced;

FIG. 18 is a sectional view schematically showing an example of the structure of a pulse wave sensor;

FIG. 19 is a sectional view schematically showing an example of the structure of a pulse wave sensor;

FIG. 20 is a circuit diagram showing a third configuration example of the filter 12;

FIG. 21 is a chart showing measurement results with a test subject walking (6 km/h);

FIG. 22 is a chart showing measurement results with a test subject jogging (8 km/h);

FIG. 23 is a chart showing measurement results with a test subject jogging (10 km/h);

FIG. 24 is a chart showing measurement results with a test subject running (12 km/h);

FIG. 25 is a chart showing measurement results with a test subject running (14 km/h);

FIG. 26 is a chart showing measurement results with a test subject running (16 km/h);

FIG. 27 is a table for comparison between constant lighting and pulse lighting;

FIG. 28 is a circuit diagram showing a configuration example of the pulse driver 17;

FIG. 29 is a schematic diagram illustrating detection (demodulation) applied to a pulse wave signal;

FIG. 30 is a chart showing the light-emission and -reception characteristics of the optical sensor 11;

FIG. 31 is a table for comparison of measurement results between an old and a new type;

FIG. 32 is a chart showing results of measurement outdoors;

FIG. 33 is a schematic diagram illustrating the principle of pulse wave measurement on the ear;

FIG. 34 is an external view of a pulse wave sensor according to a third embodiment of the present invention;

FIG. 35 is a block diagram of a pulse wave sensor according to the third embodiment;

FIG. 36A is a front view schematically showing an example of how an earphone 1X of a first design is worn on the outer ear E;

FIG. 36B is a front view schematically showing an example of how an earphone 1X of a second design is worn on the outer ear E;

FIG. 36C is a front view schematically showing an example of how an earphone 1X of a third design is worn on the outer ear E;

FIG. 36D is a front view schematically showing an example of how an earphone 1X of a fourth design is worn on the outer ear E;

FIG. 37 is a system diagram showing a modified example (an earplug structure) of a pulse wave sensor;

FIG. 38 is a system diagram showing an example of application to a hearing aid;

FIG. 39 is a block diagram showing a configuration example of a sleep sensor;

FIG. 40 is a schematic diagram showing a configuration example of a home appliance control system employing the sleep sensor 501;

FIG. 41A is a schematic diagram showing a first example of how the sleep sensor 501 (of a forehead-worn type) is worn;

FIG. 41B is a schematic diagram showing a second example of how the sleep sensor 501 (of an ear-worn type) is worn;

FIG. 42 is an exterior view of a pulse wave sensor according to a fourth embodiment of the present invention:

FIG. 43 is a schematic diagram showing a first compression method of the damping member 630;

FIG. 44 is a schematic diagram showing a second compression method of the damping member 630;

FIG. 45 is a schematic diagram showing a third compression method of the damping member 630;

FIG. 46 is a schematic diagram showing a fourth compression method of the damping member 630;

FIG. 47 is a chart showing measurement results with no earpiece, with no sponge, with a test subject traveling at 8 km/h;

FIG. 48 is a chart showing measurement results with no earpiece, with no sponge, with a test subject traveling at 12 km/h;

FIG. 49 is a chart showing measurement results with no earpiece, with no sponge, with a test subject traveling at 16 km/h;

FIG. 50 is a chart showing measurement results with an earpiece, with no sponge, with a test subject traveling at 8 km/h;

FIG. 51 is a chart showing measurement results with an earpiece, with no sponge, with a test subject traveling at 12 km/h;

FIG. 52 is a chart showing measurement results with an earpiece, with no sponge, with a test subject traveling at 16 km/h;

FIG. 53 is a chart showing measurement results with an earpiece, with a 1 cm thick sponge, with a test subject traveling at 8 km/h;

FIG. 54 is a chart showing measurement results with an earpiece, with a 1 cm thick sponge, with a test subject traveling at 12 km/h;

FIG. 55 is a chart showing measurement results with an earpiece, with a 1 cm thick sponge, with a test subject traveling at 16 km/h;

FIG. 56 is a chart showing measurement results with an earpiece, with a 2 cm thick sponge, with a test subject traveling at 8 km/h;

FIG. 57 is a chart showing measurement results with an earpiece, with a 2 cm thick sponge, with a test subject traveling at 12 km/h;

FIG. 58 is a chart showing measurement results with an earpiece, with a 2 cm thick sponge, with a test subject traveling at 16 km/h;

FIG. 59 is a table summarizing measurement results;

FIG. 60 is an external view showing a first modified example of the fourth embodiment;

FIG. 61 is an external view showing a second modified example of the fourth embodiment; and

FIG. 62 is an external view showing a third modified example of the fourth embodiment.

### DESCRIPTION OF EMBODIMENTS

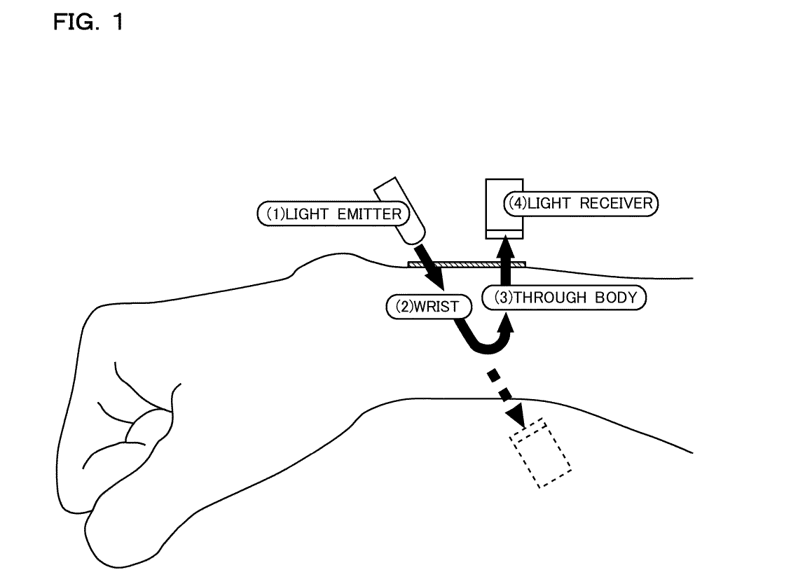

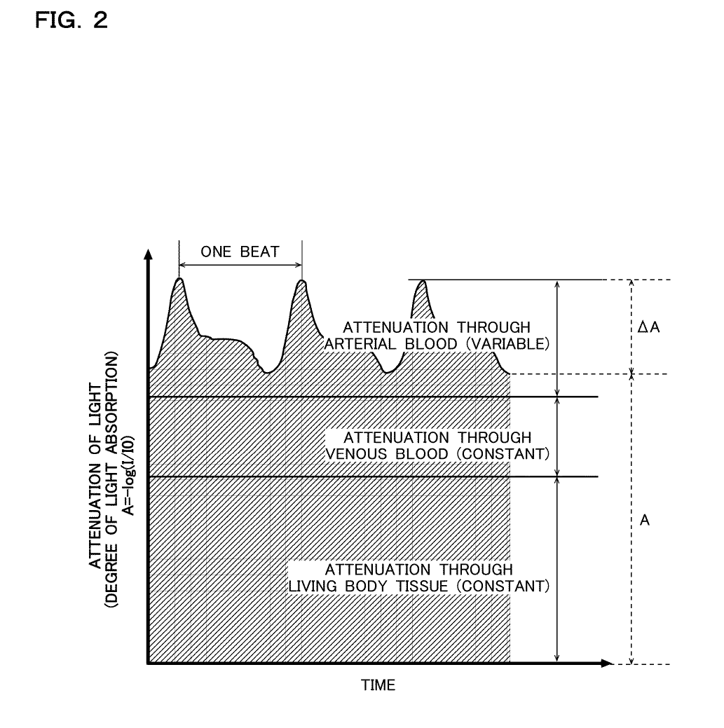

FIG. 1 is a schematic diagram illustrating the principle of pulse measurement on the wrist, and FIG. 2 is a waveform chart showing how the level of light attenuation (the degree of light absorption) in the living body varies with time.

In pulse measurement by volume pulse wave monitoring (plethysmography), for example, as shown in FIG. 1, a part (in FIG. 1, a wrist) of the living body pressed against a measurement window is irradiated with light emitted from a light emitter (such as an LED (light-emitting diode), and the intensity of the light that emerges out of the living body after passing through it is detected by a light receiver (such as a photodiode or a phototransistor). Here, as shown in FIG. 2, while the level of light attenuation (the degree of light absorption) in living body tissue and in venous blood (ascribable to deoxygenated hemoglobin Hb) is constant, the level of light attenuation (the degree of light absorption) in arterial blood (ascribable to oxygenated hemoglobin HbO₂) is variable along the time axis due to pulsation. Accordingly, the so-called “living body window” present in a visible to near-infrared region of the spectrum (the wavelength region in which light passes through the living body easily) can be exploited to measure change in the degree of light absorption in peripheral arteries, and in this way it is possible to measure volume pulse waves on a non-invasive basis.

Although in FIG. 1, for the sake of simple illustration, the pulse wave sensor (the light emitter and the light receiver) is shown to be worn on the back (outside) of the wrist, this is not meant to limit the wearing position of the pulse wave sensor; the pulse wave sensor may be worn on the front (inside) of the wrist or on any other part (e.g., on the tip of a finger, on the third joint of a finger, on the forehead, between the eyebrows (on the glabella), on the tip of the nose, on a cheek, under an eye, on a temple, or on an earlobe).

### <What Pulse Waves Reveal>

Pulse waves are under the control of the heart and the autonomic nerve system; thus they do not always behave steadily but exhibit different variations (fluctuations) according to the state of a test subject. Thus, by analyzing variations (fluctuations) in pulse waves, it is possible to acquire various kinds of information on the physical condition of the test subject. For example, the heart rate reveals the test subject's motor ability, mental tenseness, etc.; variations in the heart rate reveal the test subject's fatigue, quality of sleep, intensity of stress, etc. Moreover, acceleration pulse waves determined by differentiating pulse waves twice with respect to the time axis reveal the test subject's blood vessel age, level of atherosclerosis, etc.

### Pulse Wave Sensor

### First Embodiment

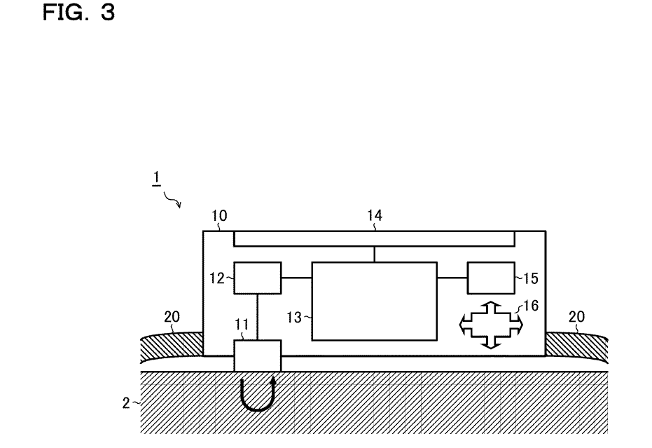

FIG. 3 is a block diagram showing a pulse wave sensor according to a first embodiment of the present invention. The pulse wave sensor 1 of the first embodiment has a bracelet structure (wrist watch structure) composed of a main unit 10 and a belt 20, the belt 20 being attached to opposite ends of the main unit 10 so as to be worn around a living body 2 (specifically, a wrist). Examples of the material for the belt 20 include leather, metal, and resin.

The main unit 10 includes an optical sensor 11, a filter 12, a controller 13, a display 14, a communicator 15, and a power supply 16.

The optical sensor 11 is provided on the reverse face of the main unit 10 (the face facing the living body 2). The living body 2 is irradiated with light from a light emitter, and the intensity of the light that has passed through the living body is detected with a light receiver; in this way, pulse wave data are acquired. In the pulse wave sensor 1 of the first embodiment, the optical sensor 11 adopts, instead of a configuration where the light emitter and the light receiver are arranged on opposite sides of the living body 2 across it (a so-called transmission type configuration; see the broken-line arrow in FIG. 1), a configuration where the light emitter and the light receiver are both arranged on the same side of the living body 2 (a so-called reflection type configuration; see the solid-line arrows in FIG. 1). The present inventors have, through experiments, confirmed that the latter configuration allows satisfactory pulse wave measurement when performed on the wrist. Specific structures of the optical sensor 11 will be described in detail later.

The filter 12 applies filtering and amplification to the output signal of the optical sensor 11 (the detection signal of the light receiver) and delivers the result to the controller 13. Specific circuit configurations of the filter 12 will be described in detail later.

The controller 13 controls the operation of the entire pulse wave sensor 1 in a concentrated fashion, and also applies various kinds of signal processing to the output signal of the filter 12 to acquire various kinds of information (fluctuations in pulse waves, heart rate, variations in heart rate, acceleration pulse waves, etc.) associated with pulse waves. The controller 13 can suitably comprise a CPU (central processing unit) or the like.

The display 14 is provided on the obverse face of the main unit 10 (the face facing away from the living body 2), and outputs display information (including date-and-time information, pulse wave measurement results, etc.). Thus, the display 14 corresponds to the dial of a wrist watch. The display 14 can suitably comprise a liquid crystal display panel or the like.

The communicator 15 transmits the measurement data of the pulse wave sensor 1 to an external device (such as a personal computer or a cellular telephone) on a wireless or wired basis. In particular, with a configuration where the measurement data of the pulse wave sensor 1 are wirelessly transmitted to an external device, there is no need for wired connection between the pulse wave sensor 1 and the external device; this makes it possible, for example, to transmit measurement data on a real-time basis without restricting the test subject's activities. In a case where the pulse wave sensor 1 is given a watertight structure, from the perspective of completely eliminating external terminals, it is preferable to adopt wireless communication for external transmission of measurement data. In a case where wireless transmission is adopted, it is possible to suitably use a wireless communication module IC complying with Bluetooth (a registered trademark) or the like.

The power supply 16 includes a battery and a DC/DC converter. The power supply 16 converts an input voltage from the battery to a desired output voltage, and feeds it to different parts of the pulse wave sensor 1. A battery-operated pulse wave sensor 1 like this requires no cable connection from an external power supply during pulse wave measurement, and thus allows pulse wave measurement without restricting the test subject's activities. As the battery, it is preferable to use a secondary battery (such as a lithium-ion secondary battery or an electric double-layer capacitor), which allows repeated recharging. A configuration employing a secondary battery as the battery eliminates the need for troublesome battery replacement, and thus helps make the pulse wave sensor 1 more convenient to use. Power feeding from outside for battery charging can be achieved by contact power feeding, such as by use of a USB (universal serial bus) cable, or by non-contact power feeding, such as by electromagnetic induction, electric-field coupling, or magnetic resonance. In a case where the pulse wave sensor 1 is given a watertight structure, from the perspective of completely eliminating external terminals, it is preferable to adopt non-contact power feeding for power feeding from outside.

With the pulse wave sensor 1 having a bracelet structure as described above, unless the test subject intentionally removes the optical sensor 11 from the wrist, the pulse wave sensor 1 is hardly likely to drop off the wrist during pulse wave measurement. Thus, it is possible to measure pulse waves without restricting the test subject's activities.

Moreover, with the pulse wave sensor 1 having a bracelet structure, the test subject is hardly conscious of wearing the pulse wave sensor 1. Thus, even in a case where pulse wave measurement lasts for a long period (several days to several months), the test subject can go through it without feeling excessive stress.

In particular, with a pulse wave sensor 1 provided with a display 14 that can display not only results of pulse wave measurement but also date-and-time information etc. (i.e., a pulse wave sensor 1 having a wrist-watch structure), the test subject can wear the pulse wave sensor 1 as a wrist watch on a day-to-day basis. Thus, it is possible to further alleviate the awkwardness from wearing the pulse wave sensor 1, and thus to develop a new group of users.

It is preferable that the pulse wave sensor 1 be given a watertight structure. Such a structure allows pulse wave measurement with no trouble resulting from the pulse wave sensor 1 getting wet with water (rain) or sweat. In a case where the pulse wave sensor 1 is shared by a number of people (e.g., when it is used as an item to rent at a sports gym), it has only to be washed whole to be kept clean.

### <Optical Sensor (Structure)>

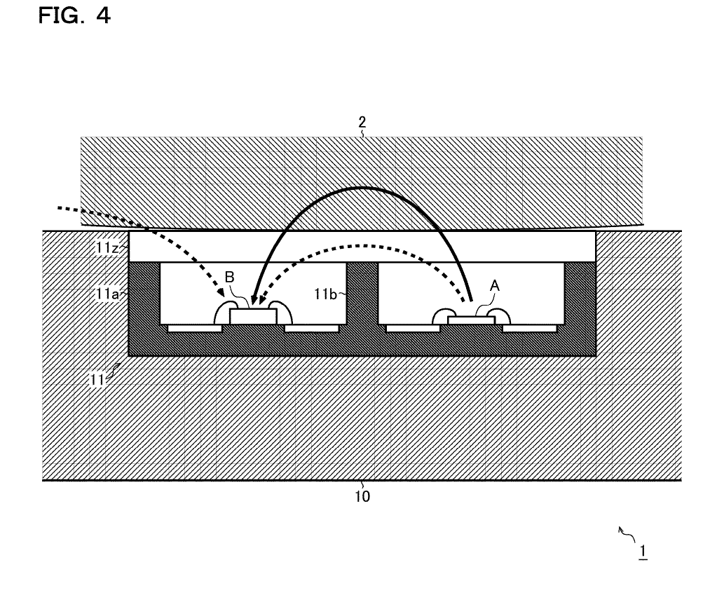

FIG. 4 is a sectional view schematically showing a first configuration example of the optical sensor 11. The optical sensor 11 of the first configuration example has a case 11a, a light-shielding wall 11b, a light-transmitting plate 11z, a light emitter x, and a light receiver y.

The case 11a is a box-shaped member in which the light emitter x and the light receiver y are housed. The case 11a is buried in the main unit 10 such that the light-transmitting plate 11z, which stops the open face of the case 11a, is flush with the obverse face of the main unit 10 (the face facing the living body 2).

The light-shielding wall 11b is a member which divides the case 11a into a first region, where the light emitter x is mounted, and a second region, where the light receiver y is mounted. Providing the light-shielding wall 11b helps prevent light from passing directly from the light emitter x to the light receiver y, and thus helps enhance the detection accuracy of pulse wave data. It is preferable that the case 11a and the light-shielding wall 11b be molded integrally.

The light-transmitting plate 11z is a light-transmitting member which stops the open face of the case 11a. Providing the light-transmitting plate 11z helps prevent soiling of the light emitter x and the light receiver y (as with dust), and thus makes it possible to use bare chips (a light-emitting and a light-emitting chip), i.e., chips that are not sealed in resin or the like, as the light emitter x and the light receiver y.

With the optical sensor 11 of the first configuration example, it is possible to acquire pulse wave data of a test subject by irradiating the living body 2 with light from the light emitter x and then detecting with the light receiver y the intensity of the light that has passed through the living body 2.

However, with the optical sensor 11 of the first configuration example, due to the presence of the light-transmitting plate 11z between, at one side, the living body 2 and, at the other side, the light emitter x and the light receiver y, light may pass directly from the light emitter x to the light receiver y through the light-transmitting plate 11z. Moreover, with the optical sensor 11 of the first configuration example, when the contact between the optical sensor 11 and the living body 2 becomes loose, outside light may leak into the light receiver y. Outside light entering the light receiver y without passing through the living body 2 lowers the detection accuracy (S/N ratio) of pulse wave data; thus, for enhanced pulse wave data detection accuracy, it is important to solve the problems mentioned just above.

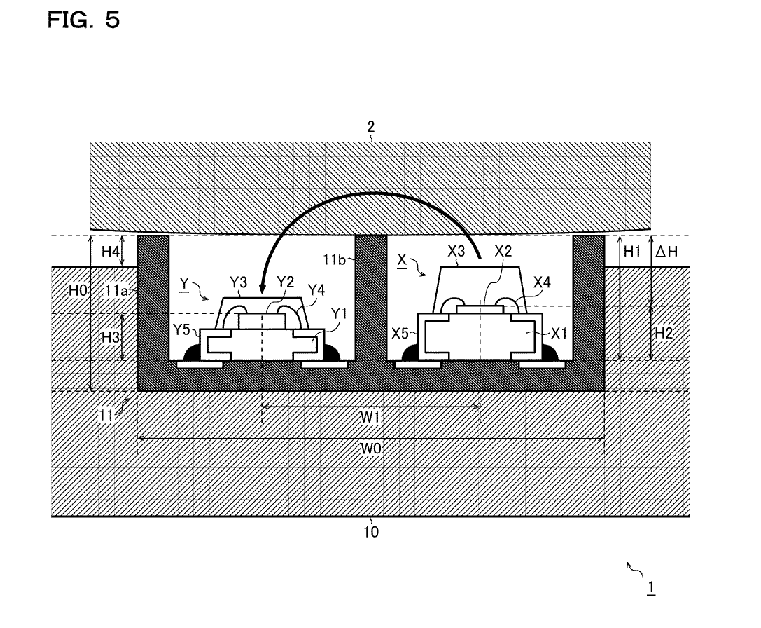

FIG. 5 is a sectional view schematically showing a second configuration example of the optical sensor 11. The optical sensor 11 of the second configuration example has a case 11a, a light-shielding wall 11b, a light emitter X, and a light receiver Y; thus, the optical sensor 11 of the second configuration example lacks the light-transmitting plate 11z present in the previous configuration example.

The case 11a is a box-shaped member in which the light emitter X and the light receiver Y are housed. The external dimensions (height H0, width W0, and depth D0) of the case 11a are, e.g., H0=1.5 mm, W0=4.5 mm, and D0=3.0 mm. The case 11a is buried in the main unit 10 such that the former protrudes from the latter by a predetermined dimension H4 (e.g., H4=0.3 mm) With this structure, the protruding part of the case 11a prevents outside light from leaking into the light receiver Y, and this helps enhance the detection accuracy of pulse wave data.

The light-shielding wall 11b is a member which divides the case 11a into a first region, where the light emitter X is mounted, and a second region, where the light receiver Y is mounted. As in the first embodiment described previously, providing the light-shielding wall 11b helps prevent light from passing directly from the light emitter X to the light receiver Y, and thus helps enhance the detection accuracy of pulse wave data. It is preferable that the case 11a and the light-shielding wall 11b be molded integrally.

The light emitter X has a substrate X1, a light-emitting chip X2, a seal X3, wires X4, and conductors X5. The substrate X1 is a member on which the light-emitting chip X2 is mounted. The light-emitting chip X2 is a light-emitting element (e.g., a bare chip of a green LED) which outputs light of a predetermined wavelength. The seal X3 is a light-transmitting member which seals the light-emitting chip X2. The wires X4 are members that electrically connect the light-emitting chip X2 to the conductors X5. The conductors X5 are electrically conductive members that are formed to extend from the top face to the bottom face of the substrate X1, and are soldered to a wiring pattern formed on the floor face of the case 11a.

The light receiver Y has a substrate Y1, a light-receiving chip Y2, a seal Y3, wires Y4, and conductors Y5. The substrate Y1 is a member on which the light-receiving chip Y2 is mounted. The light-receiving chip Y2 is a photoelectric conversion element (e.g., a bare chip of a phototransistor sensitive to light in a near-infrared to visible region of the spectrum) which converts light in a predetermined wavelength region into an electrical signal. The seal Y3 is a light-transmitting member which seals the light-receiving chip Y2. The wires Y4 are members that electrically connect the light-receiving chip Y2 to the conductors Y5. The conductors Y5 are electrically conductive members that are formed from the top face to the bottom face of the substrate Y1, and are soldered to a wiring pattern formed on the floor face of the case 11a.

Thus, in the optical sensor 11 of the second configuration example, used as the light emitter X and the light receiver Y are not bare chips but packaged semiconductor devices. Accordingly, there is no need to stop the open face of the case 11a with a transparent plate. Thus, it is possible to prevent light from passing directly from the light emitter X to the light receiver Y through a transparent plate, and thus to enhance the detection accuracy of pulse wave data.

Moreover, in the optical sensor 11 of the second configuration example, between the height H1 of the light-shielding wall 11b and the height H2 of the light emitter X, the relationship H1>H2 holds. Here, the height H1 of the light-shielding wall 11b refers to the distance from the floor face of the case 11a to the top end of the light-shielding wall 11b (e.g., H1=1.4 mm) On the other hand, the height H2 of the light emitter X refers to the distance from the floor face of the case 11a to the light emission face of the light-emitting chip X2 (e.g., H2=0.5 mm) However, considering that the light-emitting chip X2 is far thinner than the substrate X1, the thickness of the substrate X1 may be taken as the height H2 of the light emitter X.

With a dimension design satisfying the above relationship, light can be blocked effectively so as not to directly pass from the light emitter X to the light receiver Y, and this helps enhance the detection accuracy of pulse wave data.

However, if the height H2 of the light emitter X is set excessively small relative to the height H1 of the light-shielding wall 11b, the light emitted from the light emitter X is scattered or attenuated before reaching the living body 2, reducing the intensity of the light detected by the light receiver Y and thus lowering the detection accuracy of pulse wave data. Thus, the offset distance ΔH (=H1−H2) calculated by subtracting the height H2 of the light emitter X from the height H1 of the light-shielding wall 11b is subject to an optimal design range.

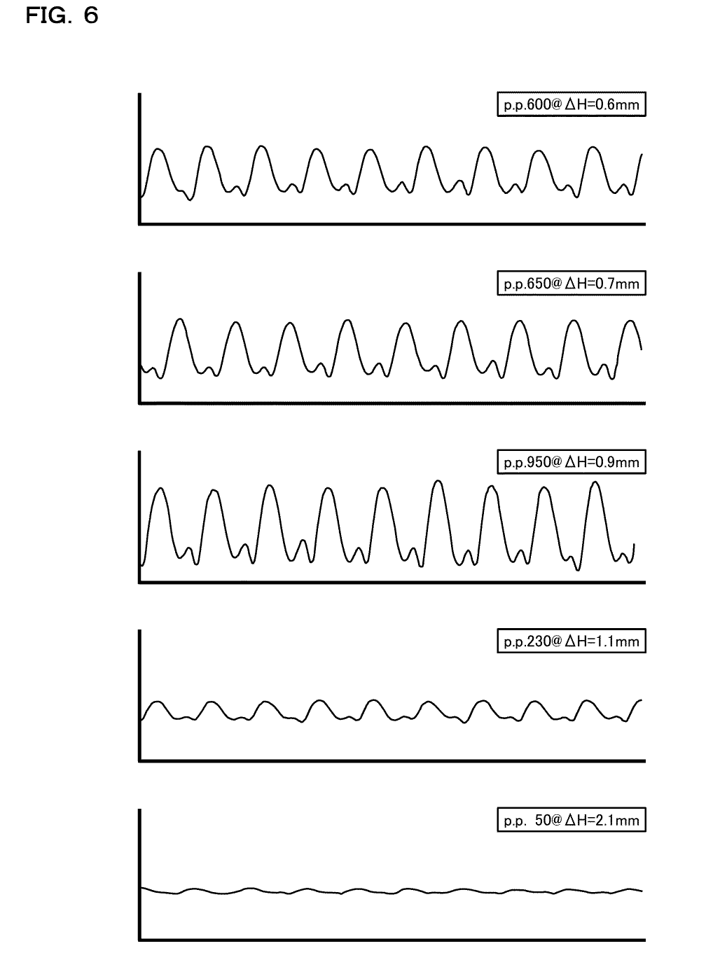

FIG. 6 is a waveform chart showing the correlation between the offset distance ΔH and the signal strength (the peak-to-peak value of the light reception signal), showing plots of the received waveform as observed when ΔH=0.6 mm, 0.7 mm, 0.9 mm, 1.1 mm, and 2.1 mm respectively, from top. FIG. 6 reveals that, when the offset distance ΔH equals 0.9 mm, the signal strength is at the maximum. From these test results, it can be concluded that it is preferable that the offset distance ΔH be in a design range of 0 mm<ΔH<2 mm (and more preferably in a design range of 0.6 mm≦ΔH≦1.4 mm).

For example, in a design where a light emitter X having a seal X3 with a thickness of 0.6 mm is used and the offset distance ΔH is set at 0.9 mm, the light emitter X can be designed to have a thickness such that the top face of the seal X3 lies at a height level 0.3 mm lower than the top end of the light-shielding wall 11b.

Moreover, in the optical sensor 11 of the second configuration example, between the height H2 of the light emitter X and the height H3 of the light receiver Y, the relationship H2>H3 holds. Here, the height H3 of the light receiver Y refers to the distance from the floor face of the case 11a to the light reception face of the light-receiving chip Y2 (e.g., H3=0.3 mm) However, considering that the light-receiving chip Y2 is far thinner than the substrate Y1, the thickness of the substrate Y1 may be taken as the height H3 of the light receiver Y.

With a dimension design satisfying the above relationship, outside light is less likely to reach the light receiver Y, and this helps enhance the detection accuracy of pulse wave data.

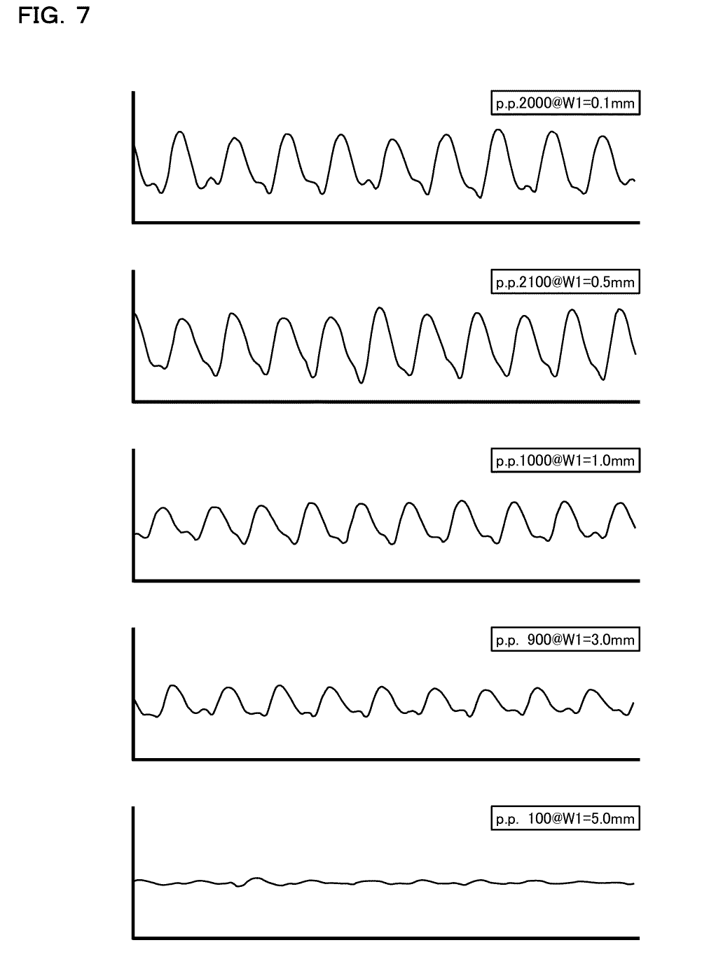

Next, with reference to FIG. 7, how the signal strength varies with the chip-to-chip distance W1 between the light emitter X and the light receiver Y will be studied. FIG. 7 is a waveform chart showing the correlation between the chip-to-chip distance W1 and the signal strength, showing plots of the received waveform as observed when W1=0.1 mm, 0.5 mm, 1.0 mm, 3.0 mm, and 5.0 mm respectively, from top. FIG. 7 reveals that, when the chip-to-chip distance W1 equals 0.5 mm, the signal strength is at the maximum. From these test results, it can be concluded that it is preferable that the chip-to-chip distance W1 be in a design range of 0.1 mm≦W1≦3.0 mm (and more preferably in a design range of 0.2 mm≦W2≦0.8 mm.

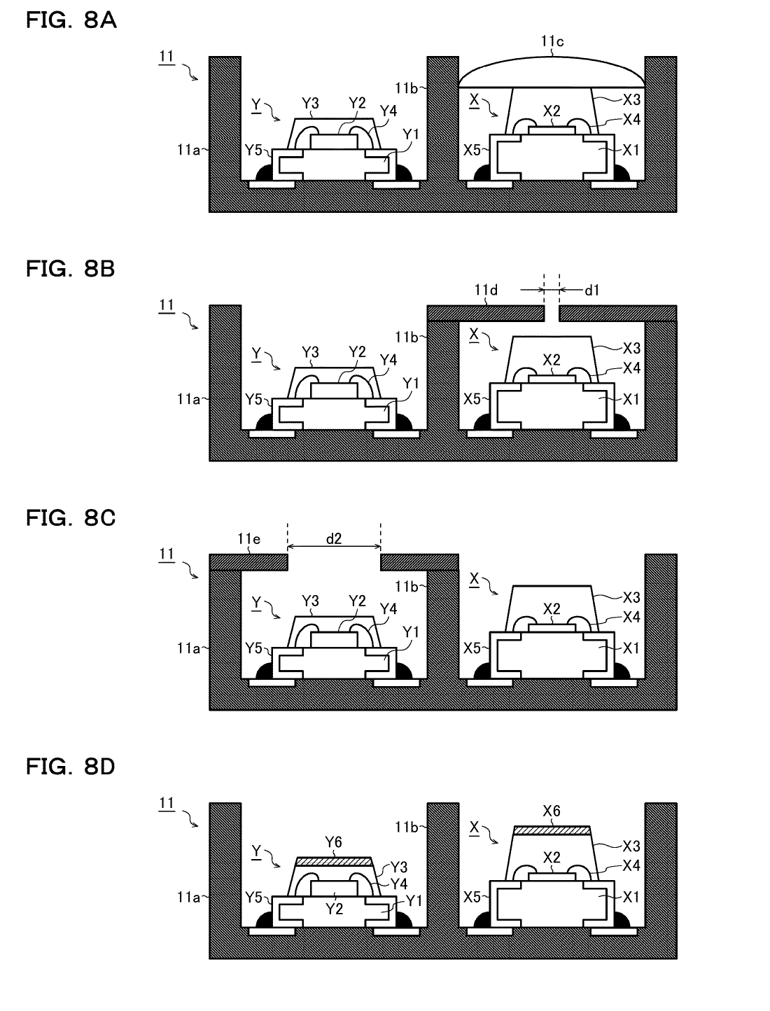

Next, with reference to FIGS. 8A to 8D, modified examples of the optical sensor 11 will be described. FIGS. 8A to 8D are sectional views schematically showing a third to a sixth configuration example, respectively, of the optical sensor 11. The third to sixth configuration examples are largely similar to the second configuration example described previously, but include different additional components for enhancement of the detection accuracy of pulse wave data.

Specifically, in the optical sensor 11 of the third configuration example (FIG. 8A), there is provided a condenser lens 11c over the light emitter X. Providing the condenser lens 11c permits the light emitted from the light emitter X to be condensed before being shone on the living body 2; this makes it possible to increase the intensity of the light detected by the light receiver Y, and thereby to enhance the detection accuracy of pulse wave data.

In the optical sensor 11 of the fourth configuration example (FIG. 8B), the first region, where the light emitter X is mounted, is covered by a lid member 11d having an opening d1 smaller than the light emission region of the light emitter X. For example, in a case where the light emission region of the light emitter X is a 0.7 mm by 0.7 mm square region, the opening d1 can be formed in the shape of a circle with a diameter of 0.5 mm or in the shape of a 0.5 mm by 0.5 mm square. Providing the lid member 11d prevents diffusion of the light emitted from the light emitter X, and prevents light from passing directly from the light emitter X to the light receiver Y; it is thus possible to enhance the detection accuracy of pulse wave data.

In the optical sensor 11 of the fifth configuration example (FIG. 8C), the second region, where the light receiver Y is mounted, is covered by a lid member 11e having an opening d2 larger than the light reception region of the light receiver Y. For example, in a case where the light reception region of the light receiver Y is a 0.7 mm by 0.7 square region, the opening d2 can be formed in the shape of a circle with a diameter of 1.0 mm or in the shape of a 1.0 mm by 1.0 mm square. Providing the lid member 11e prevents outside light from leaking into the light receiver Y, and thus it is possible to enhance the detection accuracy of pulse wave data.

In the optical sensor 11 of the sixth configuration example (FIG. 8D), at least one of the light emitter X and light receiver Y has a color filter X6 or Y6 which selectively transmits a predetermined wavelength component (around the peak output wavelength of the light emitter X). Providing the color filter X6 or Y6 makes it possible to remove unnecessary wavelength components, and thus to enhance the detection accuracy of pulse wave data.

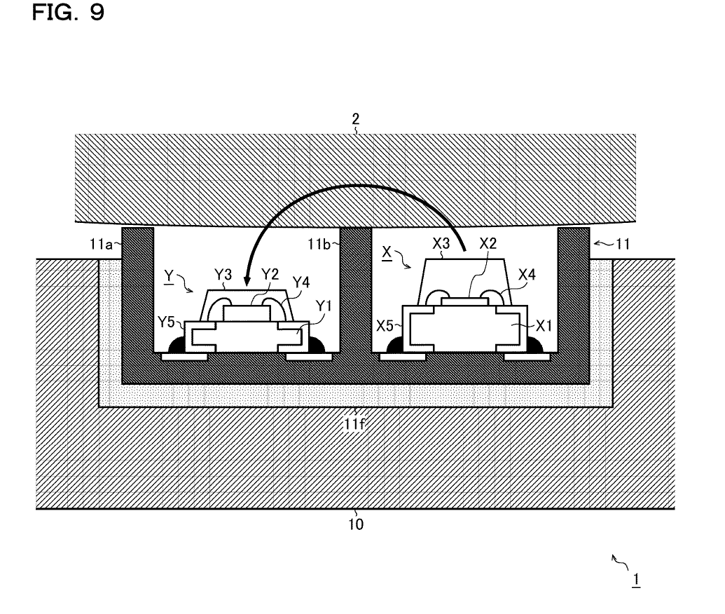

Next, with reference to FIG. 9, yet another modified example of the optical sensor 11 will be described. FIG. 9 is a sectional view schematically showing a seventh configuration example of the optical sensor 11. The seventh configuration example is largely similar to the second configuration example described previously, but is more elaborately configured for enhancement of the detection accuracy of pulse wave data.

The optical sensor 11 of the seventh configuration example has a damping member 11f between the main unit 10 and the case 11a. As the damping member 11f, rubber, synthetic sponge, or the like can be suitably used. This structure helps achieve closer contact between the optical sensor 11 and the living body 2, and thus makes it possible to measure pulse waves stably.

The additional components in the third to sixth configuration examples (FIGS. 8A to 8D) and the seventh configuration example (FIG. 9) may each be implemented singly, or may be implemented in any combination.

### <Optical Sensor (Arrangement)>

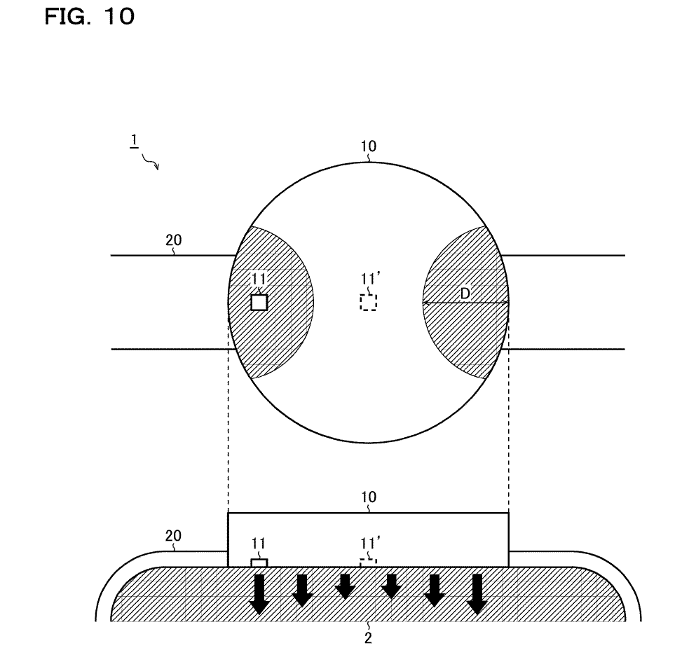

FIG. 10 is a layout diagram showing the arrangement of an optical sensor 11 in a wrist watch-type pulse wave sensor 1. In the wrist watch-type pulse wave sensor 1, an optical sensor 11 is held in a main unit 10 (e.g., with a diameter of 28 mm), and a belt 20 is connected to opposite ends of the main unit 10. When the wrist watch-type pulse wave sensor 1 is worn on a living body 2 (wrist), a pressing force (see the bold arrows in FIG. 10) is applied to the living body 2 as the belt 20 is tightened.

With respect to such wrist watch-type pulse wave sensors 1, the present inventors have found that the pressing force applied from the main unit 10 to the living body 2 has a particular distribution pattern so that, according to the arrangement position of the optical sensor 11, the closeness of contact between the optical sensor 11 and the living body 2 (and hence the signal strength of the light reception signal) varies.

Through intensive studies, the present inventors have found out the following: it is possible to enhance the signal strength of the light reception signal by arranging the optical sensor 11 near the force application point where the pressing force applied to the living body 2 is strongest, more specifically, inside the region (the hatched region in FIG. 10) where D≦10 mm holds, with D representing the distance from the connection point between the main unit 10 and the belt 20 to the arrangement position of the optical sensor 11 (the center position of the optical sensor 11).

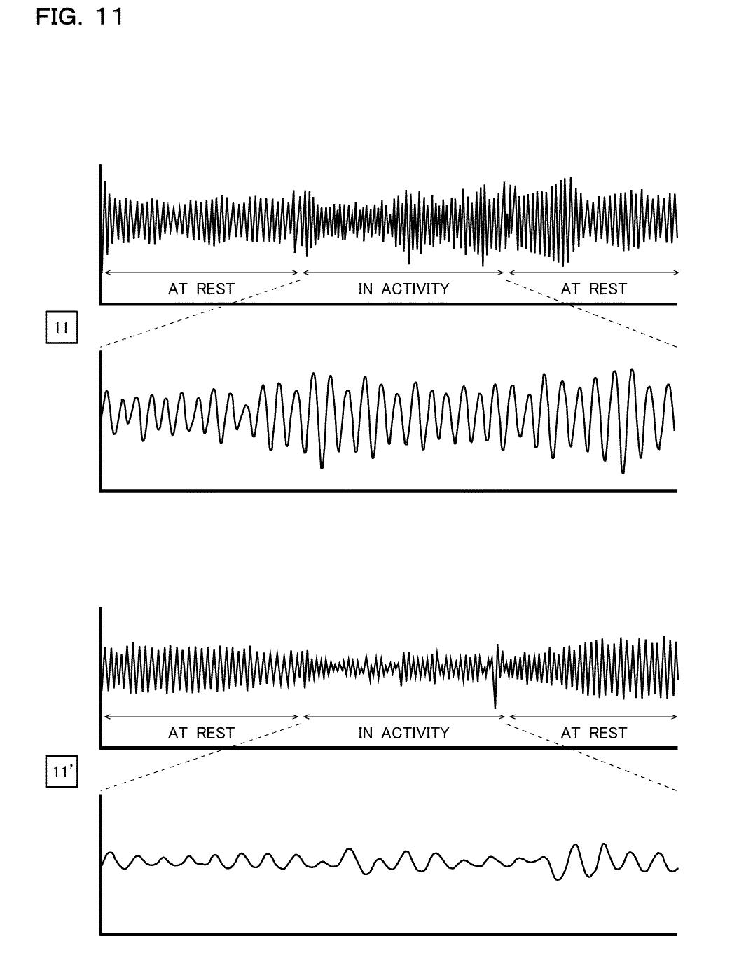

FIG. 11 is a waveform chart showing the correlation between the arrangement of the optical sensor 11 and the signal strength. The upper half shows a plot of the light reception signal of the optical sensor 11 arranged in an end part of the main unit 10 (inside the hatched region in FIG. 10), and the lower half shows a plot of the light reception signal of the optical sensor 11 arranged in a central part of the main unit 10 (outside the hatched region in FIG. 10). A comparison of the two plots will reveal that, with the optical sensor 11 arranged in an end part of the main unit 10, owing to the enhanced closeness of contact with the living body 2, it is possible to measure pulse waves accurately not only with the test subject at rest but also with the test subject in activity.

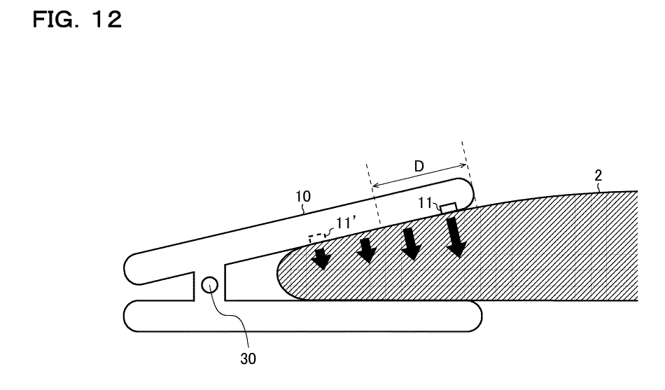

The above finding applies not only to a wrist watch-type pulse wave sensor 1 but also to an earring-type pulse wave sensor 1 as shown in FIG. 12.

FIG. 12 is a layout diagram showing the arrangement of an optical sensor 11 in an earring-type pulse wave sensor 1. In the earring-type pulse wave sensor 1, an optical sensor 11 is held in a main unit 10 (e.g., with a total length of 24 mm from a first end to a second end), with a spring hinge 30 connected to the first end and the second end left as an open end. The main unit 10 is a member which, when the earring-type pulse wave sensor 1 is worn on a living body 2 (earlobe), is given a pressing force toward the living body 2 (see the bold arrows in FIG. 12) by the spring hinge 30.

Here, the force application point where the pressure toward the living body 2 is strongest is the second end (open end) of the main unit 10. Accordingly, by arranging the optical sensor 11 inside the region where D≦10 mm holds, with D representing the distance from the second end (open end) of the main unit 10 to the arrangement position of the optical sensor 11 (the center position of the optical sensor 11), it is possible to enhance the closeness of contact between the optical sensor 11 and the living body 2, and thereby to enhance the signal strength of the light reception signal.

Although FIGS. 10 and 12 show, as an example, a configuration where a single optical sensor 11 is provided on the obverse face of the main unit 10, this is not meant to limit the number of optical sensors 11 provided; a plurality of optical sensors 11 may be provided inside a region near the force application point where the pressing force toward the living body 2 is strongest.

### <Filter>

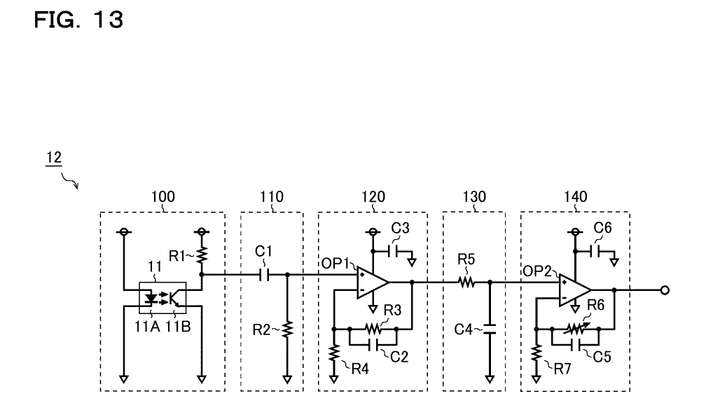

FIG. 13 is a circuit diagram showing a first configuration example of the filter 12. The filter 12 of the first configuration example has a current/voltage converter circuit 100, a first-order CR high-pass filter circuit 110 (hereinafter referred to as the HPF (high-pass filter) circuit 110), an amplifier circuit 120, a first-order CR low-pass filter circuit 130 (hereinafter referred to as the LPF (low-pass filter) circuit 130), and an amplifier circuit 140.

The current/voltage converter circuit 100 is a circuit which converts a current signal output from the optical sensor 11 into a voltage signal, and includes a resistor R1 (e.g., 200 kΩ). An anode of a light-emitting diode 11A provided in the optical sensor 11 is connected to a node to which a supply voltage VDD is applied (a supply voltage VDD application node). A cathode of the light-emitting diode 11A is connected to a node at a ground voltage (a ground node). A collector of a phototransistor 11B provided in the optical sensor 11 is connected via the resistor R1 to a supply voltage VDD application node. An emitter of the phototransistor 11B is connected to a ground node.

The HPF circuit 110 is a circuit which eliminates a low-frequency component superimposed on the output signal of the current/voltage converter circuit 100, and includes a capacitor C1 (e.g., 0.1 μF) and a resistor R2 (e.g., 4.7 MΩ). A first terminal of the capacitor C1 is connected to the collector of the phototransistor 11B. A second terminal of the capacitor C1 is connected via the resistor R2 to a ground node. The HPF circuit 110 configured as described above is designed to have a cut-off frequency of 0.34 Hz.

The amplifier circuit 120 is a circuit which amplifies the output signal of the HPF circuit 110, and includes an operational amplifier OP1, a resistor R3 (e.g., 100 kΩ), a resistor R4 (e.g., 10 kΩ), a capacitor C2 (e.g., 0.01 ΩF), and a capacitor C3 (e.g., 0.1 μF). A non-inverting input terminal (+) of the operational amplifier OP1 is connected to the second terminal of the capacitor C1. An inverting input terminal (−) of the operational amplifier OP1 is connected via the resistor R3 to an output terminal of the operational amplifier OP1, and is also connected via the resistor R4 to a ground node. A first power terminal of the operational amplifier OP1 is connected to a supply voltage VDD application node. A second power terminal of the operational amplifier OP1 is connected to a ground node. The capacitor C2 is connected in parallel with the resistor R3. The capacitor C3 is connected between the first power terminal of the operational amplifier OP1 and a ground node.

The LPF circuit 130 is a circuit which eliminates a high-frequency component superimposed on the output signal of the amplifier circuit 120, and includes a resistor R5 (e.g., 100 kΩ) and a capacitor C4 (e.g., 1.0 μF). A first terminal of the resistor R5 is connected to the output terminal of the operational amplifier OP1. The first terminal of the resistor R5 is connected to the output terminal of the operational amplifier OP1. The second terminal of the resistor R5 is connected via the capacitor C4 to a ground node. The LPF circuit 130 configured as described above is designed to have a cut-off frequency of 1.6 Hz.

The amplifier circuit 140 is a circuit which amplifies the output signal of the LPF circuit 130, and includes an operational amplifier OP2, a variable resistor R6 (e.g., 500 kΩ), a resistor R7 (e.g., 10 kΩ), a capacitor C5 (e.g., 0.01 μF), and a capacitor C6 (e.g., 0.1 μF). A non-inverting input terminal (+) of the operational amplifier OP2 is connected to the second terminal of the resistor R5. An inverting input terminal (−) of the operational amplifier OP2 is connected via the variable resistor R6 to an output terminal of the operational amplifier OP2, and is also connected via the resistor R7 to a ground node. A first power terminal of the operational amplifier OP2 is connected to a supply voltage VDD application node. A second power terminal of the operational amplifier OP2 is connected to a ground node. The capacitor C5 is connected in parallel with the variable resistor R6. The capacitor C6 is connected between the first power terminal of the operational amplifier OP2 and a ground node.

With the filter 12 of the first configuration example, it is possible, with a simple circuit configuration, to eliminate noise components superimposed on the output signal of the optical sensor 11, and thereby to enhance the detection accuracy of pulse wave data.

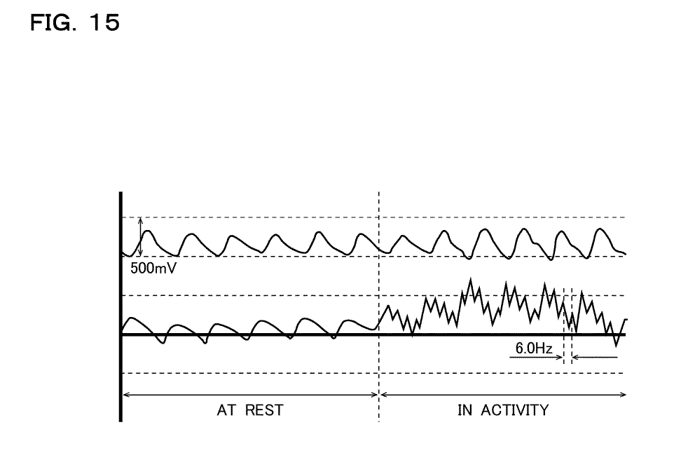

However, the filter 12 of the first configuration example sometimes cannot sufficiently eliminate the test subject's body motion noise (a noise component of about 6.0 Hz due to the test subject's motion), and thus leaves room for further improvement for high-accuracy detection of pulse waves on a test subject in activity (see the lower half of FIG. 15).

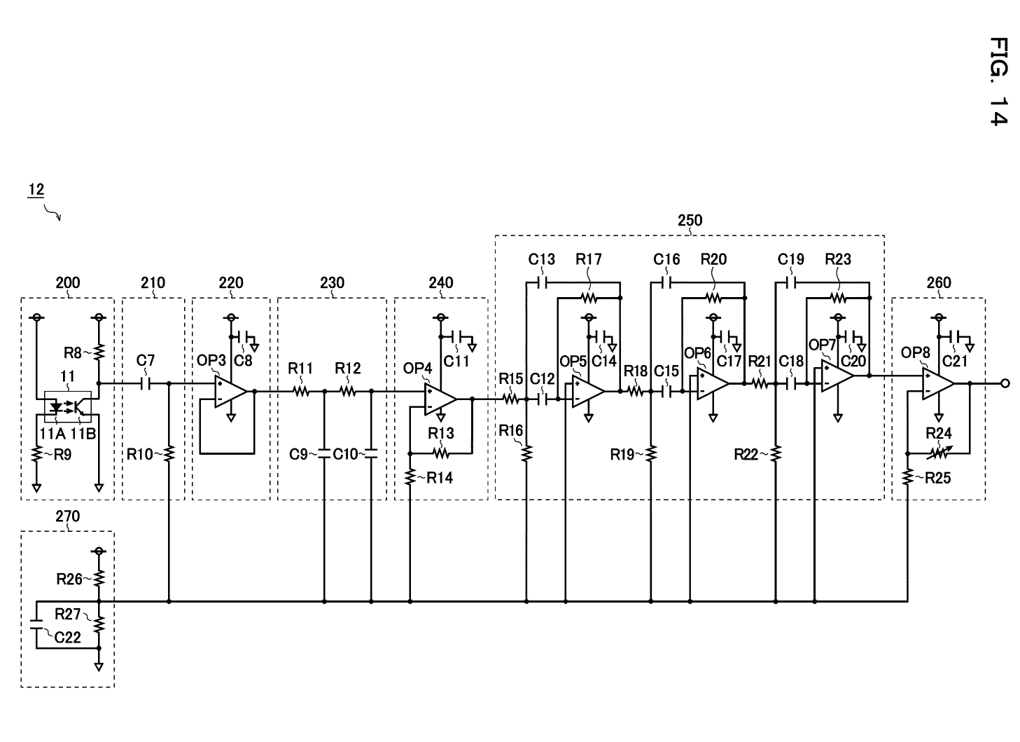

FIG. 14 is a circuit diagram showing a second configuration example of the filter 12. The filter 12 of the second configuration example has a current/voltage converter circuit 200, a first-order CR high-pass filter circuit 210 (hereinafter referred to as the HPF circuit 210), a voltage follower circuit 220, a second-order CR low-pass filter circuit 230 (hereinafter referred to as the LPF circuit 230), an amplifier circuit 240, a sixth-order band-pass filter circuit 250 (hereinafter referred to as the BPF (band-pass filter) circuit 250), an amplifier circuit 260, and an intermediate voltage generator circuit 270.

The current/voltage converter circuit 200 is a circuit which converts a current signal output from the optical sensor 11 into a voltage signal, and includes a resistor R8 (e.g., 200 kΩ) and a resistor R9 (e.g., 430Ω). An anode of a light-emitting diode 11A provided in the optical sensor 11 is connected to a node to which a supply voltage VDD is applied (a supply voltage VDD application node). A cathode of the light-emitting diode 11A is connected via the resistor R9 to a node at a ground voltage (a ground node). A collector of a phototransistor 11B provided in the optical sensor 11 is connected via the resistor R8 to a supply voltage VDD application node. An emitter of the phototransistor 11B is connected to a ground node.

The HPF circuit 210 is a circuit which eliminates a low-frequency component superimposed on the output signal of the current/voltage converter circuit 200, and includes a capacitor C7 (e.g. 1.0 μF) and a resistor R10 (e.g., 240 kΩ). A first terminal of the capacitor C7 is connected to the collector of the phototransistor 11B. A second terminal of the capacitor C7 is connected via the resistor R10 to a node to which an intermediate voltage VM is applied (an intermediate voltage VM application node). The HPF circuit 210 configured as described above is designed to have a cut-off frequency of 0.66 Hz.

The voltage follower circuit 220 is a circuit which delivers the output signal of the HPF circuit 110 to a succeeding stage, and includes an operational amplifier OP3 and a capacitor C8 (e.g., 0.1 μF). A non-inverting input terminal (+) of the operational amplifier OP3 is connected to the second terminal of the capacitor C7. An inverting input terminal (−) of the operational amplifier OP3 is connected to an output terminal of the operational amplifier OP3. A first power terminal of the operational amplifier OP3 is connected to a supply voltage VDD application node. A second power terminal of the operational amplifier OP3 is connected to a ground node. The capacitor C8 is connected between the first power terminal of the operational amplifier OP3 and a ground node.

The LPF circuit 230 is a circuit which eliminates a high-frequency component superimposed on the output signal of the voltage follower circuit 220, and includes a resistor R11 (e.g. 620 kΩ), a resistor R12 (e.g., 620 kΩ), a capacitor C9 (e.g., 1.0 μF), and a capacitor C10 (e.g., 0.1 μF). A first terminal of the resistor R11 is connected to the output terminal of the operational amplifier OP3. A second terminal of the resistor R11 is connected to a first terminal of the resistor R12, and is also connected via the capacitor C9 to an intermediate voltage VM application node. A second terminal of the resistor R12 is connected via the capacitor C10 to an intermediate voltage VM application node. The LPF circuit 230 configured as described above is designed to have a cut-off frequency of 0.26 Hz.

The amplifier circuit 240 is a circuit which amplifies the output signal of the LPF circuit 230, and includes an operational amplifier OP4, a resistor R13 (e.g., 10 kΩ), a resistor R14 (e.g., 1 kΩ), and a capacitor C11 (e.g., 0.1 μF). A non-inverting input terminal (+) of the operational amplifier OP4 is connected to the second terminal of the resistor R12. An inverting input terminal (−) of the operational amplifier OP4 is connected via the resistor R13 to an output terminal of the operational amplifier OP4, and is also connected via the resistor R14 to an intermediate voltage VM application node. A first power terminal of the operational amplifier OP4 is connected to a supply voltage VDD application node. A second power terminal of the operational amplifier OP4 is connected to a ground node. The capacitor C11 is connected between the first power terminal of the operational amplifier OP4 and a ground node.

The BPF circuit 250 is a circuit which eliminates both a low-frequency component and a high-frequency component superimposed on the output signal of the amplifier circuit 240, and includes operational amplifiers OP5 to OP7, a resistor R15 (e.g., 75 kΩ), a resistor R16 (e.g., 2 MΩ), a resistor R17 (e.g., 150 kΩ), a resistor R18 (e.g., 130 kΩ), a resistor R19 (e.g., 91 kΩ), a resistor R20 (e.g., 620 kΩ), a resistor R21 (e.g., 43 kΩ), a resistor R22 (e.g., 30 kΩ), a resistor R23 (e.g., 200 kΩ), a capacitor C12 (e.g., 1 μF), a capacitor C13 (e.g., 1 μF), a capacitor C14 (e.g., 0.1 μF), a capacitor C15 (e.g., 1 μF), a capacitor C16 (e.g., 1 μF), a capacitor C17 (e.g., 0.1 μF), a capacitor C18 (e.g., 1 μF), a capacitor C19 (e.g., 1 μF), and a capacitor C20 (e.g., 0.1 μF).

A first terminal of the resistor R15 is connected to the output terminal of the operational amplifier OP4. A second terminal of the resistor R15 is connected via the resistor R16 to an intermediate voltage VM application node. A non-inverting input terminal (+) of the operational amplifier OP5 is connected to an intermediate voltage VM application node. An inverting input terminal (−) of the operational amplifier OP5 is connected via the capacitor C12 to the second terminal of the resistor R15, and is also connected via the resistor R17 to an output terminal of the operational amplifier OP5. A first power terminal of the operational amplifier OP5 is connected to a supply voltage VDD application node. A second power terminal of the operational amplifier OP5 is connected to a ground node. The capacitor C13 is connected between the second terminal of the resistor R15 and the output terminal of the operational amplifier OP5. The capacitor C14 is connected between the first power terminal of the operational amplifier OP5 and a ground node.

A first terminal of the resistor R18 is connected to the output terminal of the operational amplifier OP5. A second terminal of the resistor R18 is connected via the resistor R19 to an intermediate voltage VM application node. A non-inverting input terminal (+) of the operational amplifier OP6 is connected to an intermediate voltage VM application node. An inverting input terminal (−) is connected via the capacitor C15 to a second terminal of the resistor R18, and is also connected via the resistor R20 to an output terminal of the operational amplifier OP6. A first power terminal of the operational amplifier OP6 is connected to a supply voltage VDD application node. A second power terminal of the operational amplifier OP6 is connected to a ground node. The capacitor C16 is connected between the second terminal of the resistor R18 and the output terminal of the operational amplifier OP6. The capacitor C17 is connected between the first power terminal of the operational amplifier OP6 and a ground node.

A first terminal of the resistor R21 is connected to the output terminal of the operational amplifier OP6. A second terminal of the resistor R21 is connected via the resistor R22 to an intermediate voltage VM application node. A non-inverting input terminal (+) of the operational amplifier OP7 is connected to an intermediate voltage VM application node. An inverting input terminal (−) of the operational amplifier OP7 is connected via the capacitor C18 to the second terminal of the resistor R21, and is also connected via the resistor R23 to an output terminal of the operational amplifier OP7. A first power terminal of the operational amplifier OP7 is connected to a supply voltage VDD application node. A second power terminal of the operational amplifier OP7 is connected between the second terminal of the resistor R21 and the output terminal of the operational amplifier OP7. The capacitor C19 is connected between the second terminal of the resistor R21 and the output terminal of the operational amplifier OP7. The capacitor C20 is connected between the first power terminal of the operational amplifier OP7 and a ground node.

The BPF circuit 250 configured as described above is designed to have a pass band of 0.80 Hz to 2.95 Hz.

The amplifier circuit 260 is a circuit which amplifies the output signal of the BPF circuit 250, and includes an operational amplifier OP8, a variable resistor R24 (e.g., 1 MΩ), a resistor R25 (e.g., 1 kΩ), and a capacitor C21 (e.g., 0.1 μF). A non-inverting input terminal (+) of the operational amplifier OP8 is connected to an output terminal of the operational amplifier OP7. An inverting input terminal (−) of the operational amplifier OP8 is connected via the variable resistor R24 to an output terminal of the operational amplifier OP8, and is also connected via the resistor R25 to an intermediate voltage VM application node. A first power terminal of the operational amplifier OP8 is connected to a supply voltage VDD application node. A second power terminal of the operational amplifier OP8 is connected to a ground node. The capacitor C21 is connected between the first power terminal of the operational amplifier OP8 and a ground node.

The intermediate voltage generator circuit 260 is a circuit which generates the intermediate voltage VM (=VDD/2) by dividing the supply voltage VDD to one-half (½), and includes a resistor R26 (e.g., 1 kΩ), a resistor R27 (e.g., 1 kΩ), and a capacitor C22 (0.1 μF). A first terminal of the resistor R26 is connected to a supply voltage VDD application node. A second terminal of the resistor R26 and a first terminal of the resistor R27 are both connected to an intermediate voltage VM application node. A second terminal of the resistor R27 is connected to a ground node. The capacitor C22 is connected in parallel with the resistor R27.

The filter 12 of the second configuration example can properly eliminate the test subject's body motion noise, and thus allows high-accuracy detection of pulse waves not only with the test subject at rest but also with the test subject in activity (e.g., while walking) (see the upper half of FIG. 15).

In the filter 12 of the second configuration example, the HPF circuit 210, the LPF circuit 230, the amplifier circuit 240, the BPF circuit 250, and the amplifier circuit 260 all operate relative to the intermediate voltage VM (VDD/2) as a reference voltage. Thus, the output signal of the filter 12 has a waveform in which the amplitude varies upward and downward relative to the intermediate voltage VM. Accordingly, with the filter 12 of the second configuration example, it is possible to accurately detect pulse wave data while preventing saturation of the output signal (its sticking to the supply voltage VDD or the ground voltage).

### Pulse Wave Sensor

### Second Embodiment

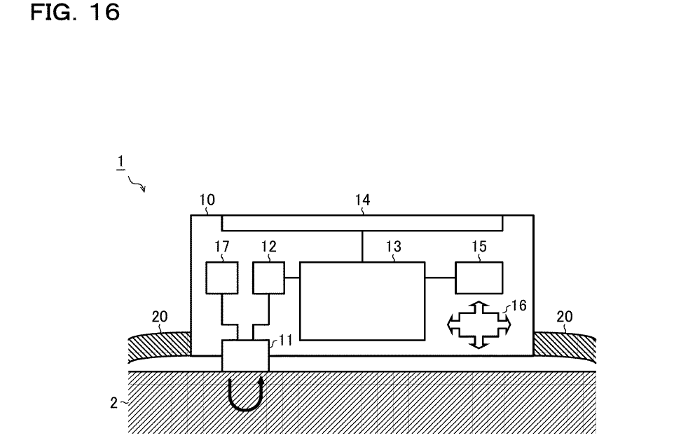

FIG. 16 is a block diagram showing a pulse wave sensor according to a second embodiment of the present invention. The pulse wave sensor 1 of the second embodiment has a configuration similar to that in the first embodiment, but is modified, to achieve higher accuracy in pulse wave measurement on a test subject in activity and outdoors, in the following aspects: a body motion suppression structure is adopted; in addition, a different driving method is adopted in the optical sensor 11. The modification in the driving method of the optical sensor 11 involves use, as the light-emitter in the optical sensor 11, of a pulse driver 17 which pulse-drives the light emitter of the optical sensor 11 with higher luminance than outside light, and incorporation of a detector circuit which applies detection (demodulation) to the output signal of the optical sensor 11. Specific configurations of the pulse driver 17 and of the filter 12 will be described in detail later.

### <Development of In-Activity Measurement Technology>

As mentioned previously, with a wrist watch-type pulse wave sensor 1, it is possible to accurately measure pulse waves not only with the test subject at rest but also when the test subject in comparatively light activity (e.g., while walking) (see the upper half of FIG. 15). However, when the test subject is in more strenuous activity (while jogging or running), body motion noise may hamper precise pulse wave measurement, leaving room for still further improvement.

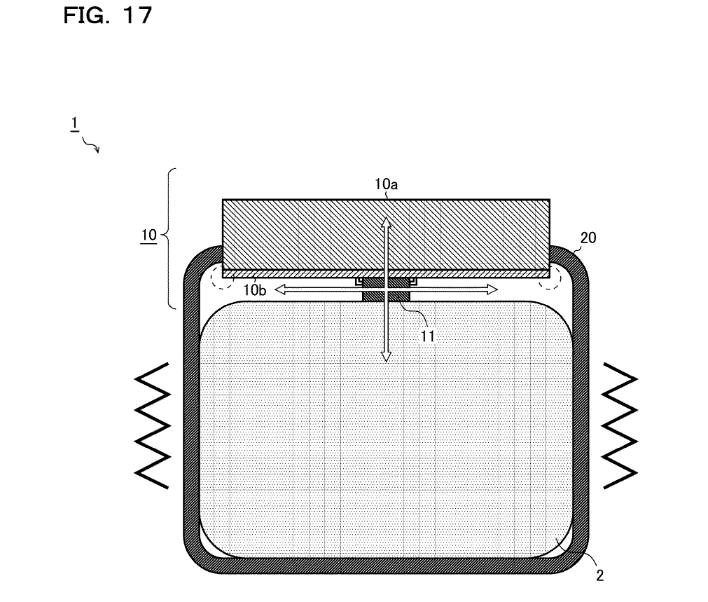

The body motion noise mentioned above will now be studied with reference to FIG. 17. FIG. 17 is a sectional view schematically showing the mechanism by which body motion noise is produced. With a pulse wave sensor 1 that does not adopt the motion noise suppression structure described below (for convenience′ sake, occasionally referred to as the old-type pulse wave sensor 1 in the following description), when a minute change in the body (such as a tension or a crease in the skin, or a motion of a muscle) resulting from the test subject's motion causes vibration to be transmitted via the belt 20 worn around the living body (wrist) 2 to the body 10a of the pulse wave sensor 1, the vibration propagates as it is, i.e., hardly attenuated, to a printed circuit board 10b attached to the body 10a. This produces a large variation in the optical distance from the optical sensor 11 mounted on the printed circuit board 10b to the living body (wrist) 2, and appears in the form of body motion noise in the output signal of the optical sensor 11.

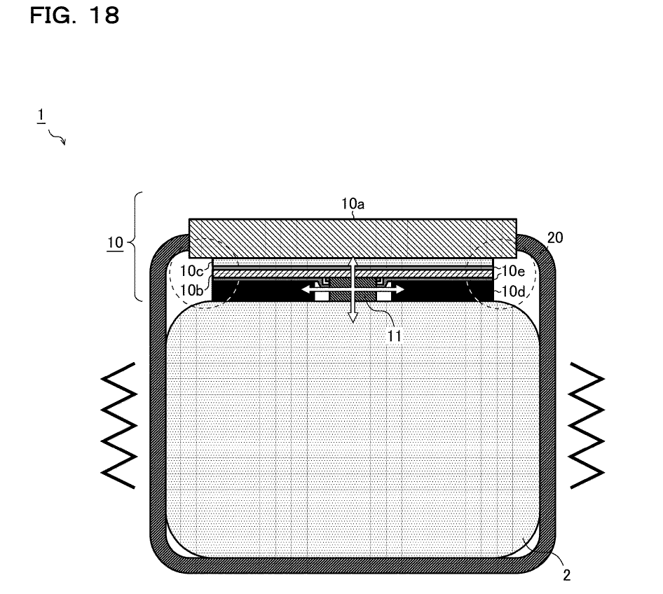

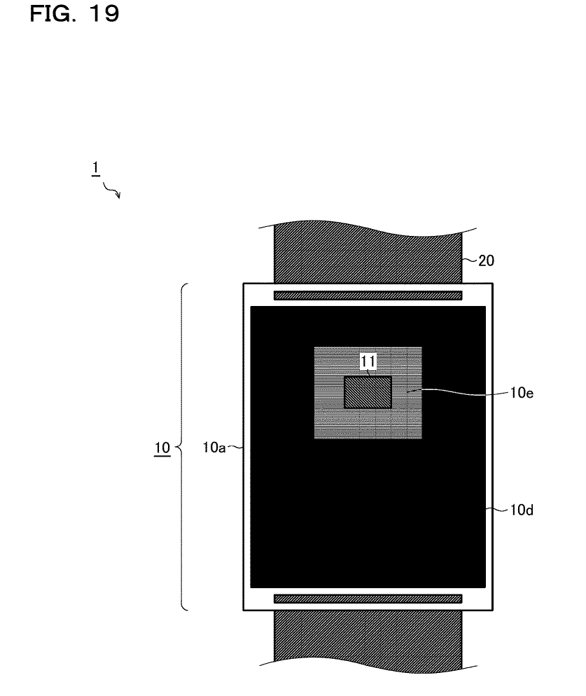

FIGS. 18 and 19 are a sectional view and a plan view, respectively, schematically showing a configuration example of a pulse wave sensor 1 that adopts a motion noise suppression structure (for convenience′ sake, occasionally referred to as the new-type pulse wave sensor 1 in the following description) (the plan view being one showing the pulse wave sensor 1 as seen from under its bottom face on which the optical sensor 11 is mounted).

In the new-type pulse wave sensor 1, the main unit 10 includes a body 10a, a printed circuit board 10b, a damping member 10c, a close-contact member 10d, and a protective member 10e.

The body 10a is a housing which holds components (such as the optical sensor 11) constituting the pulse wave sensor 1. A belt 20 is attached to opposite ends of the body 10a, and is worn around a living body (wrist) 2. It is preferable to give the body 10a a low-center-of-gravity structure by avoiding a multiple-layer structure or by arranging in a part close to the living body (wrist) 2 a member with a comparatively large weight (such as a battery). With a low-center-of-gravity structure, the body 10a is less likely to vibrate even when the test subject is in activity; this helps reduce variation in the optical distance from the optical sensor 11 to the living body (wrist) 2, and thus helps reduce body motion noise.

The printed circuit board 10b is a member on which electronic circuit components such as the optical sensor 11 are mounted, and is attached to the bottom face of the body 10a (the face facing the living body (wrist) 2). The printed circuit board 10b is designed in a size smaller than the body 10a as seen in a plan view so that the belt 20 and the printed circuit board 10b are attached to the body 10a with such a gap (about 5 mm) left in between as to prevent mutual contact. With this structure, even when the test subject is in activity, vibration is less likely to propagate directly from the belt 20 to the printed circuit board 10b; this helps reduce variation in the optical distance between the optical sensor 11 and the living body (wrist) 2, and thus helps reduce body motion noise.

The damping member 10c is a highly vibration-absorbent (flexible, or elastic) member which is provided between the printed circuit board 10b and the body 10a (hence between the optical sensor 11 and the body 10a). Usable for the damping member 10c is a gel material such as a shock-absorbent gel, or sponge or rubber. Providing the damping member 10c helps alleviate propagation of vibration from the body 10a to the optical sensor 11; this helps reduce variation in the optical distance between the optical sensor 11 and the living body (wrist) 2, and thus helps reduce body motion noise.

The close-contact member 10d is a highly close-contact member which is provided around the optical sensor 11 to keep it in close contact with the living body (wrist) 2. Usable as the close-contact member 10d is double-sided adhesive tape or an adhesive pad. The close-contact member 10d is designed to have a thickness approximately equal to or somewhat smaller than that of the optical sensor 11. Providing the close-contact member 10d helps improve the closeness of contact between the optical sensor 11 and the living body (wrist) 2; this helps reduce variation in the optical distance between the optical sensor 11 and the living body (wrist) 2, and thus helps reduce body motion noise. It is preferable that the close-contact member 10d be arranged with a gap (about 5 mm) left from the optical sensor 11. With this structure, it is easier for the optical sensor 11 to receive the light returning from the living body (wrist) 2, and this helps enhance the accuracy of pulse wave measurement. The close-contact member 10d also functions as a light-shielding member for preventing outside light from leaking into the optical sensor 11. From the viewpoint of the light-shielding function, it is preferable that the close-contact member 10d be black in color to absorb light more easily.

The protective member 10e is a member which covers at least one of the obverse and reverse faces of the printed circuit board 10b to protect electronic circuit components (such as the optical sensor 11) from impact and soiling. Usable as the protective member 10e is electrically insulating tape or a resin coating. Like the close-contact member 10d, it is preferable that the protective member 10e be black in color.

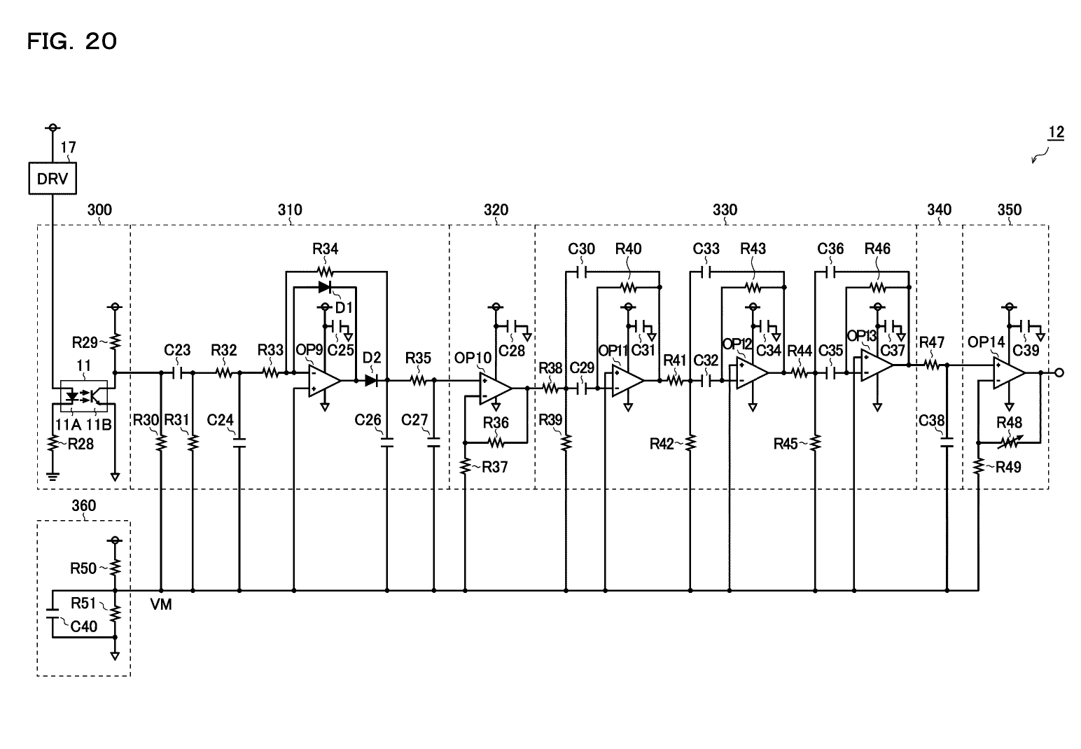

FIG. 20 is a circuit diagram of a third configuration example of the filter 12. The filter 12 of the third configuration example has a current/voltage converter circuit 300, a detector circuit 310, an amplifier circuit 320, a sixth-order operational amplifier multiple-feedback band-path filter 330 (hereinafter referred to as the BPF (band-pass filter) circuit 330), a first-order low-pass filter circuit 340 (hereinafter referred to as the LPF (low-pass filter) circuit 340), an amplifier circuit 350, and an intermediate voltage generator circuit 360.

The current/voltage converter circuit 300 is a circuit which converts a current signal output from the optical sensor 11 into a voltage signal, and includes a resistor R28 (e.g., 430Ω) and a resistor R29 (e.g., 200 kΩ). An anode of a light-emitting diode 11A (corresponding to the light emitter) provided in the optical sensor 11 is connected via a pulse driver 17 to a node to which a supply voltage VDD (e.g., +3.3 V) is applied (a supply voltage VDD application node). A cathode of the light-emitting diode 11A is connected via the resistor R28 to a node to which a ground voltage GND2 is applied (a ground voltage GND2 application node). A collector of a phototransistor 11B (corresponding to the light receiver) provided in the optical sensor 11 is connected via the resistor R29 to a supply voltage VDD application node. An emitter of the phototransistor 11B is connected to a node to which a ground voltage GND is applied (a ground voltage GND application node).

The detector circuit (demodulator circuit) 310 is a circuit which applies detection (demodulation) to the output signal of the current/voltage converter circuit 300, and includes an operational amplifier OP9, a resistor R30 (e.g., 10 kΩ), a resistor R31 (e.g., 160 kΩ), a resistor R32 (e.g., 16 kΩ), a resistor R33 (e.g., 10 kΩ), a resistor R34 (e.g., 10 kΩ), a resistor R35 (e.g., 620 kΩ), a capacitor C23 (e.g., 1.0 μF), a capacitor C24 (e.g., 10 nF), a capacitor C25 (e.g., 0.1 μF), a capacitor C26 (e.g., 1.0 μF), a capacitor C27 (e.g., 1.0 μF), and diodes D1 and D2. A collector of the phototransistor 11B is connected via the resistor R30 to a node to which an intermediate voltage VM is applied (an intermediate voltage VM application node). A first terminal of the capacitor C23 is connected to the collector of the phototransistor 11B. A second terminal of the capacitor C23 is connected via the resistor R31 to an intermediate voltage VM application node. A first terminal of the resistor R32 is connected to the second terminal of the capacitor C23. A second terminal of the resistor R32 is connected via the capacitor C24 to an intermediate voltage VM application node. An inverting input terminal (−) of the operational amplifier OP9 is connected via the resistor R33 to the second terminal of the resistor R32. A non-inverting input terminal (+) of the operational amplifier OP9 is connected to an intermediate voltage VM application node. A first power terminal of the operational amplifier OP9 is connected to a supply voltage VDD application node. A second power terminal of the operational amplifier OP9 is connected to a ground voltage GND application node. An anode of the diode D1 and a first terminal of the resistor R34 are both connected to the inverting input terminal (−) of the operational amplifier OP9. A cathode of the diode D1 and an anode of the diode D2 are both connected to an output terminal of the operational amplifier OP9. A second terminal of the resistor R34 is connected to a cathode of the diode D2. The capacitor C25 is connected between the first power terminal of the operational amplifier OP9 and a ground voltage GND application node. The capacitor C26 is connected between the cathode of the diode D2 and an intermediate voltage VM application node. A first terminal of the resistor R35 is connected to the cathode of the diode D2. A second terminal of the resistor R35 is connected via the capacitor C27 to an intermediate voltage VM application node. The operation of the detector circuit 310, along with the operation of the pulse driver 17, will be described in detail later.

The amplifier circuit 320 is a circuit which amplifies the output signal of the detector circuit 310, and includes an operational amplifier OP10, a resistor R36 (e.g., 100 kΩ), a resistor R37 (e.g., 10 kΩ), and a capacitor C28 (e.g., 0.1 μF). A non-inverting input terminal (+) of the operational amplifier OP10 is connected to the second terminal of the resistor R35. An inverting input terminal (−) of the operational amplifier OP10 is connected via the resistor R36 to an output terminal of the operational amplifier OP10, and is also connected via the resistor R37 to an intermediate voltage VM application node. A first power terminal of the operational amplifier OP10 is connected to a supply voltage VDD application node. A second power terminal of the operational amplifier OP10 is connected to a ground voltage GND application node. The capacitor C28 is connected between the first power terminal of the operational amplifier OP10 and a ground voltage GND application node.

The BPF circuit 330 is a circuit which eliminates both a low-frequency component and a high-frequency component from the output signal of the amplifier circuit 320, and includes operational amplifiers OP11 to OP13, a resistor R38 (e.g., 75 kΩ), a resistor R39 (e.g., 2 MΩ), a resistor R40 (e.g., 150 kΩ), a resistor R41 (e.g., 130 kΩ), a resistor R42 (e.g., 91 kΩ), a resistor R43 (e.g., 620 kΩ), a resistor R44 (e.g., 43 kΩ), a resistor R45 (e.g., 30 kΩ), a resistor R46 (e.g., 200 kΩ), a capacitor C29 (e.g., 1.0 μF), a capacitor C30 (e.g., 1.0 μF), a capacitor C31 (e.g., 0.1 μF), a capacitor C32 (e.g., 1.0 μF), a capacitor C33 (e.g., 1.0 μF), a capacitor C34 (e.g., 0.1 μF), a capacitor C35 (e.g., 1.0 μF), a capacitor C36 (e.g., 1.0 μF), and a capacitor C37 (e.g., 0.1 μF).

A first terminal of the resistor R38 is connected to the output terminal of the operational amplifier OP10. A second terminal of the resistor R38 is connected via the resistor R39 to an intermediate voltage VM application node. A non-inverting input terminal (+) of the operational amplifier OP11 is connected to an intermediate voltage VM application node. An inverting input terminal (−) of the operational amplifier OP11 is connected via the capacitor C29 to the second terminal of the resistor R38, and is also connected via the resistor R40 to an output terminal of the operational amplifier OP11. A first power terminal of the operational amplifier OP11 is connected to a supply voltage VDD application node. A second power terminal of the operational amplifier OP11 is connected to a ground voltage GND application node. The capacitor C30 is connected between the second terminal of the resistor R38 and the output terminal of the operational amplifier OP11. The capacitor C31 is connected between the first power terminal of the operational amplifier OP11 and a ground voltage GND application node.

A first terminal of the resistor R41 is connected to the output terminal of the operational amplifier OP11. A second terminal of the resistor R41 is connected via the resistor R42 to an intermediate voltage VM application node. A non-inverting input terminal (+) of the operational amplifier OP12 is connected to an intermediate voltage VM application node. An inverting input terminal (−) of the operational amplifier OP12 is connected via the capacitor C32 to the second terminal of the resistor R41, and is also connected via the resistor R43 to an output terminal of the operational amplifier OP12. A first power terminal of the operational amplifier OP12 is connected to a supply voltage VDD application node. A second power terminal of the operational amplifier OP12 is connected to a ground voltage GND application node. The capacitor C33 is connected between the second terminal of the resistor R41 and the output terminal of the operational amplifier OP12. The capacitor C34 is connected between the first power terminal of the operational amplifier OP12 and a ground voltage GND application node.

A first terminal of the resistor R44 is connected to the output terminal of the operational amplifier OP12. A second terminal of the resistor R44 is connected via the resistor R45 to an intermediate voltage VM application node. A non-inverting input terminal (+) of the operational amplifier OP13 is connected to an intermediate voltage VM application node. An inverting input terminal (−) of the operational amplifier OP13 is connected via the capacitor C35 to the second terminal of the resistor R44, and is also connected via the resistor R46 to an output terminal of the operational amplifier OP13. A first power terminal of the operational amplifier OP13 is connected to a supply voltage VDD application node. A second power terminal of the operational amplifier OP13 is connected to a ground voltage GND application node. The capacitor C36 is connected between the second terminal of the resistor R44 and the output terminal of the operational amplifier OP13. The capacitor C37 is connected between the first power terminal of the operational amplifier OP13 and a ground voltage GND application node.

The operational amplifier multiple-feedback BPF circuit 330 configured as described above has a pass band of 0.7 Hz to 3.0 Hz.

The LPF circuit 340 is a circuit which eliminates a high-frequency component from the output signal of the BPF circuit 330, and includes a resistor R47 (e.g., 110 kΩ) and a capacitor C38 (e.g., 1.0 μF). A first terminal of the resistor R47 is connected to the output terminal of the operational amplifier OP13. A second terminal of the resistor R47 is connected via the capacitor C38 to an intermediate voltage VM application node. The LPF circuit 340 configured as described above is designed to have a cut-off frequency of 1.45 Hz.

The amplifier circuit 350 is a circuit which amplifies the output signal of the LPF circuit 340, and includes an operational amplifier OP14, a variable resistor R48 (e.g., 1 MΩ), a resistor R49 (e.g., 1 kΩ), and a capacitor C39 (e.g., 0.1 μF). A non-inverting input terminal (+) of the operational amplifier OP14 is connected to the second terminal of the resistor R47. An inverting input terminal (−) of the operational amplifier OP14 is connected via the variable resistor R48 to an output terminal of the operational amplifier OP14, and is also connected via the resistor R49 to an intermediate voltage VM application node. A first power terminal of the operational amplifier OP14 is connected to a supply voltage VDD application node. A second power terminal of the operational amplifier OP14 is connected to a ground voltage GND application node. The capacitor C39 is connected between the first power terminal of the operational amplifier OP14 and a ground voltage GND application node.

The intermediate voltage generator circuit 360 is a circuit which generates the intermediate voltage VM (=VDD/2) by dividing the supply voltage VDD to one-half, and includes a resistor R50 (e.g., 1 kΩ), a resistor R51 (e.g., 1 kΩ), and a capacitor C40 (1.0 μF). A first terminal of the resistor R50 is connected to a supply voltage VDD application node. A second terminal of the resistor R50 and a first terminal of the resistor R51 are both connected to an intermediate voltage VM application node. A second terminal of the resistor R51 is connected to a ground voltage GND application node. The capacitor C40 is connected in parallel with the resistor R51.

With the filter 12 of the third configuration example, it is possible to effectively eliminate body motion noise from the output signal (pulse wave data) of the optical sensor 11.

In the filter 12 of the third configuration example, the detector circuit 310, the amplifier circuit 320, the BPF circuit 330, the LPF circuit 340, and the amplifier circuit 350 all operate relative to the intermediate voltage VM (=VDD/2) as a reference voltage, and thus the output signal of the filter 12 has a waveform in which the amplitude varies upward and downward relative to the intermediate voltage VM. Accordingly, with the filter 12 of the third configuration example, it is possible to accurately detect pulse wave data while preventing saturation of the output signal (its sticking to the supply voltage VDD or the ground voltage GND).

With the new-type pulse wave sensor 1 that adopts a combination of the body motion noise suppression structure (FIGS. 18 and 19) and the filter 12 (FIG. 20) described above, it is possible to detect pulse waves with high accuracy not only with the test subject at rest but also with the test subject is in activity (while walking, jogging, or running).

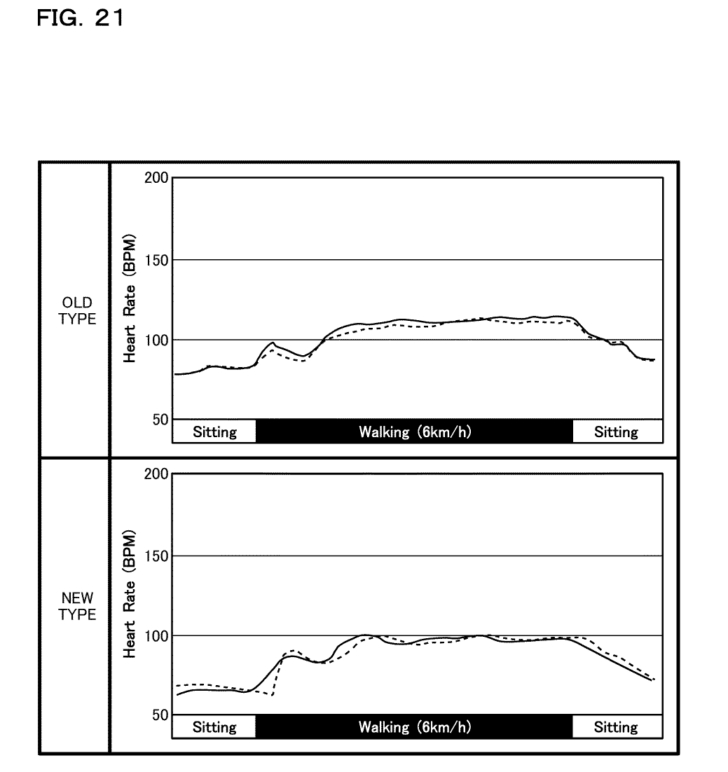

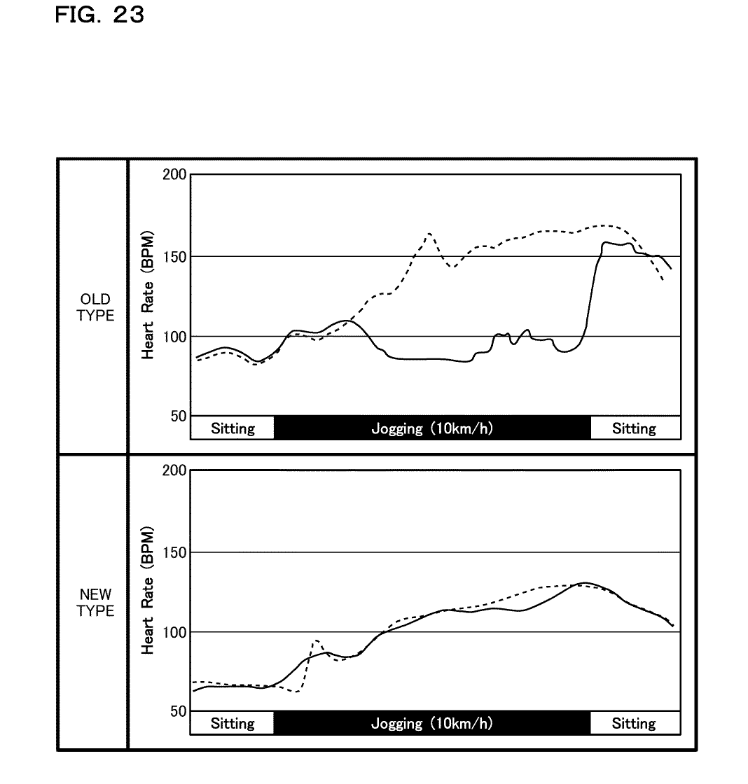

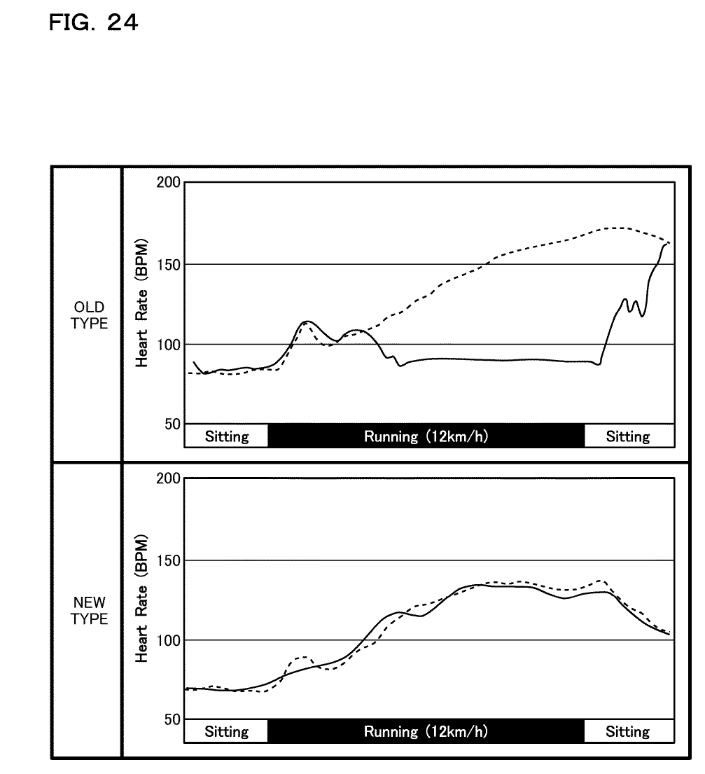

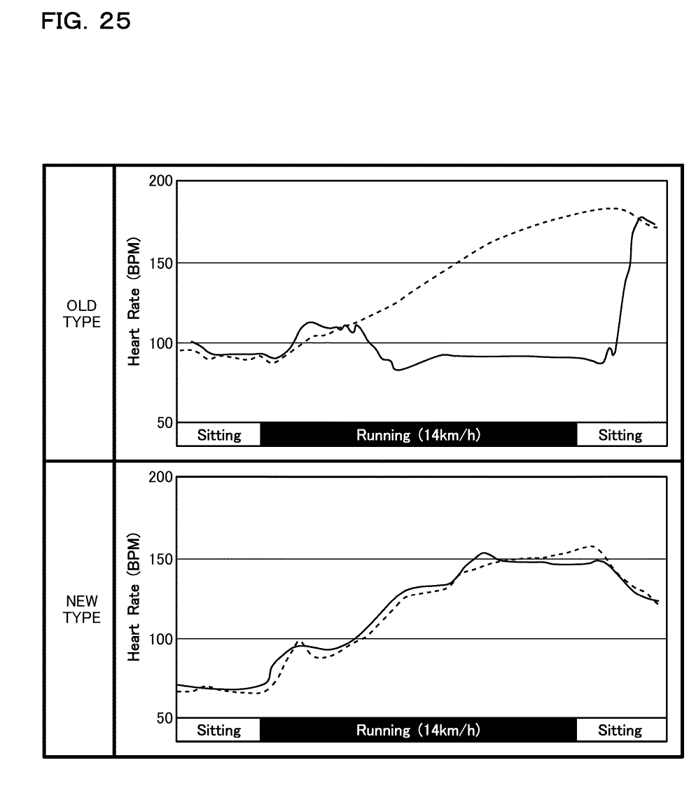

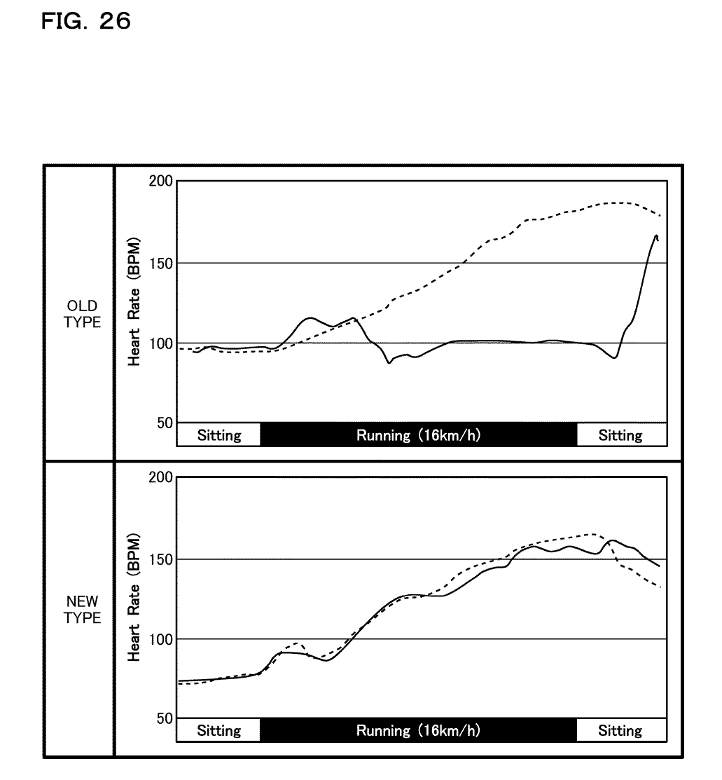

FIGS. 21 to 26 are charts showing results of measurement (of each figure, the upper half showing a plot with the old type and the lower half showing a plot with the new type) with the test subject walking (6 km/h), jogging (8 km/h and 10 km/h), and running (12 km/h and 14 km/h) respectively. In the charts, solid lines represent measurement results with the pulse wave sensor 1 (either new-type or old-type), and broken lines represent, for comparison, measurement results with a heart rate meter (commercially available) of a type that is worn using a chest belt. The activities (walking, jogging, and running) mentioned above were all done indoors, on a treadmill.

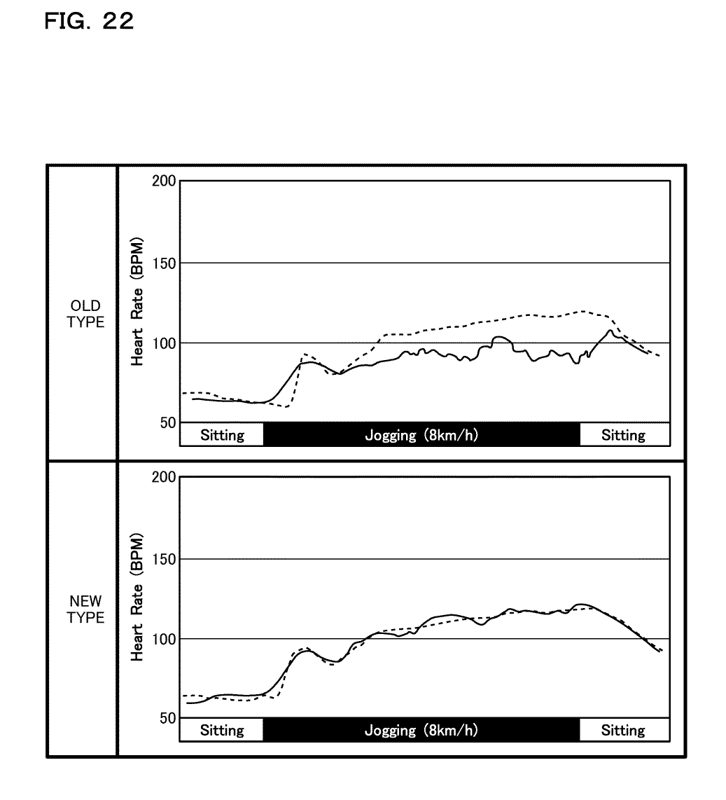

With the test subject walking, the old-type pulse wave sensor 1 yielded measurement results that exhibited a correlation with those obtained with the chest belt-worn heart rate meter (the upper half of FIG. 21). In contrast, with the test subject jogging or running, the influence of body motion noise was so great that the old-type pulse wave sensor 1 yielded measurement results that deviated from those obtained with the chest belt-worn heart rate meter (the upper half of each of FIGS. 22 to 26).

In contrast, the new-type pulse wave sensor 1 was confirmed to yield measurement results that exhibited a correlation with those obtained with the chest belt-worn heart rate meter not only with the test subject walking but also with the test subject jogging or running (the lower half of each of FIGS. 21 to 26).

### <Development of Outdoor Measurement Technology>

To allow accurate pulse wave measurement outdoors (in sunlight, which acts as extraneous disturbing light), the new-type pulse wave sensor 1 described above has the pulse driver 17 which pulse-drives the light emitter (the light-emitting diode 11A) in the optical sensor 11 with higher luminance than outside light, and in addition the filter 12 includes the detector circuit 230 which applies detection to the output signal of the optical sensor 11 to extract a pulse wave signal (see FIG. 20 referred to previously).

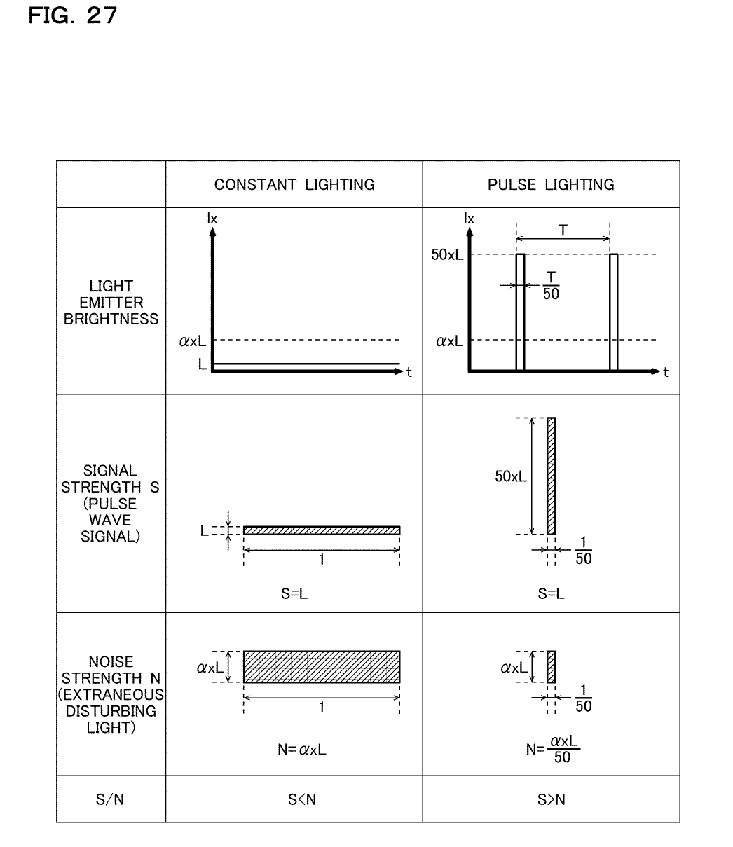

Now, the significance of changing the lighting method of the optical sensor 11 from constant lighting from pulse lighting (duty driving) will be described in detail with reference to FIG. 27. FIG. 27 is a table of comparison between constant lighting and pulse lighting, and shows, from top, the luminance of the light emitter, the signal strength S (pulse wave signal), the noise strength N (extraneous disturbing light), and the S/N (signal-to-noise) ratio.

In constant lighting, the signal strength S per unit time is given by, when the brightness of the light emitter equals L (e.g., driven at 1.5 mA), S=L (=L×1). On the other hand, the noise strength N is given by, when the brightness of extraneous disturbing light equals (α×L), N=(α×L). Accordingly, when α>1, the noise strength N is higher than the signal strength S (S<N); thus, it is not possible to obtain a satisfactory S/N ratio.

By contrast, in pulse lighting (e.g., at a driving frequency of 100 Hz and a duty ratio of 1/50), the signal strength S per unit time is given by, when the brightness of the light emitter equals (50×L) (e.g., driven at 75 mA), S=L (=(50×L)×(1/50)). On the other hand, the noise strength N is given by, when the brightness of extraneous disturbing light equals (α×L), N=(α×L)/50. In this way, by combining pulse lighting with higher luminance in the light emitter, it is possible, while keeping the signal strength S at a level comparable with that conventionally obtained, to reduce the noise strength N in accordance with the duty ratio in the light emitter, and as a result it is possible to improve the S/N ratio. The duty ratio can be set at 1/10 to 1/100, and it is preferable that the duty ratio be set at, e.g., 1/50 as mentioned above. When the duty ratio is set at 1/10, the brightness of the light emitter can be set at (10×L); when the duty ratio is set at 1/100, the brightness of the light emitter can be set at (100×L).

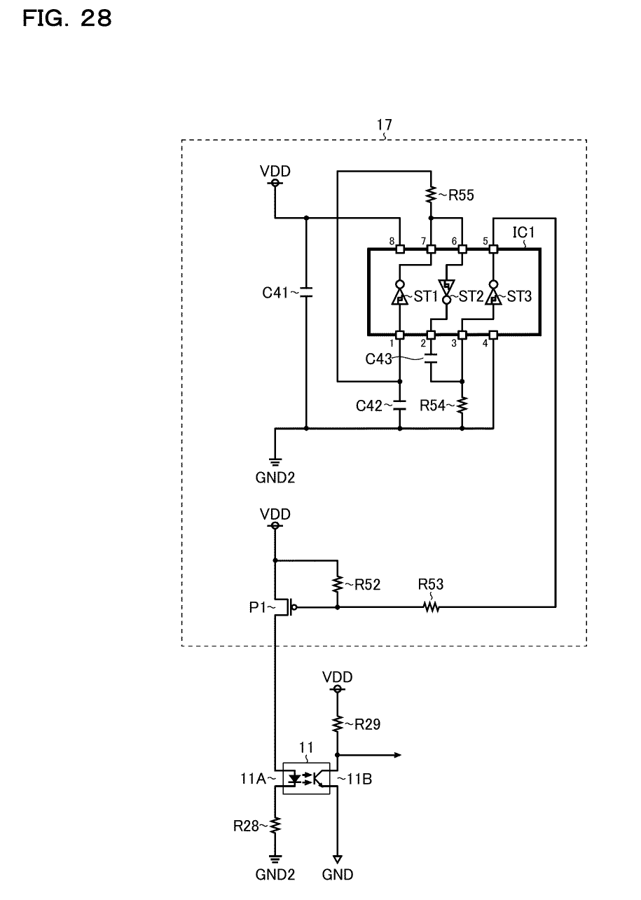

FIG. 28 is a circuit diagram showing a configuration example of the pulse driver 17. The pulse driver 17 of this configuration example includes a semiconductor device IC1, a P-channel MOS (metal oxide semiconductor) field-effect transistor P1, resistors R52 to R55, and capacitors C41 to C43.

The semiconductor device IC1 has three Schmitt triggers ST1 to ST3 and eight external terminals (pin-1 to pin-8). Pin-1 is connected to an input terminal of the Schmitt trigger ST1. Pin-2 is connected to an output terminal of the Schmitt trigger ST2. Pin-3 is connected to an input terminal of the Schmitt trigger ST3. Pin-4 is a ground terminal, and is connected, outside the semiconductor device IC1, to a node to which a ground voltage GND2 is applied (a ground voltage GND2 application node). Pin-5 is connected to an output terminal of the Schmitt trigger ST3. Pin-6 is connected to an input terminal of the Schmitt trigger ST2. Pin-7 is connected to an output terminal of the Schmitt trigger ST1. Pin-8 is a power terminal, and is connected, outside the semiconductor device IC1, to a node to which a supply voltage VDD is applied (a supply voltage VDD application node).

A source of the transistor P1 is connected to a supply voltage VDD application node. A drain of the transistor is connected to the anode of the light-emitting diode 11A. A gate of the transistor P1 is connected via the resistor R52 to a supply voltage VDD application node, and is also connected via the resistor R53 to pin-5 of the semiconductor device IC1. A first terminal of the resistor R54 is connected to pin-3 of the semiconductor device IC1. A second terminal of the resistor R54 is connected to a ground voltage GND2 application node. A first terminal of the resistor R55 is connected to pin-1 of the semiconductor device IC1. A second terminal of the resistor R55 is connected to pin-6 and pin-7 of the semiconductor device IC1. The capacitor C41 is connected between a supply voltage VDD application node and a ground voltage GND2 application node. The capacitor C42 is connected between pin-1 of the semiconductor device IC1 and a ground voltage GND2 application node. The capacitor C43 is connected between pin-2 and pin-3 of the semiconductor device IC1.

The pulse driver 17 configured as described above repeats turning on and off the transistor P1 at a predetermined driving frequency and a predetermined duty ratio to pulse-drive the current through the light-emitting diode 11A in the optical sensor 11. Used as the light-emitting diode 11A is a high-luminance device (with a peak forward current of 100 mA).

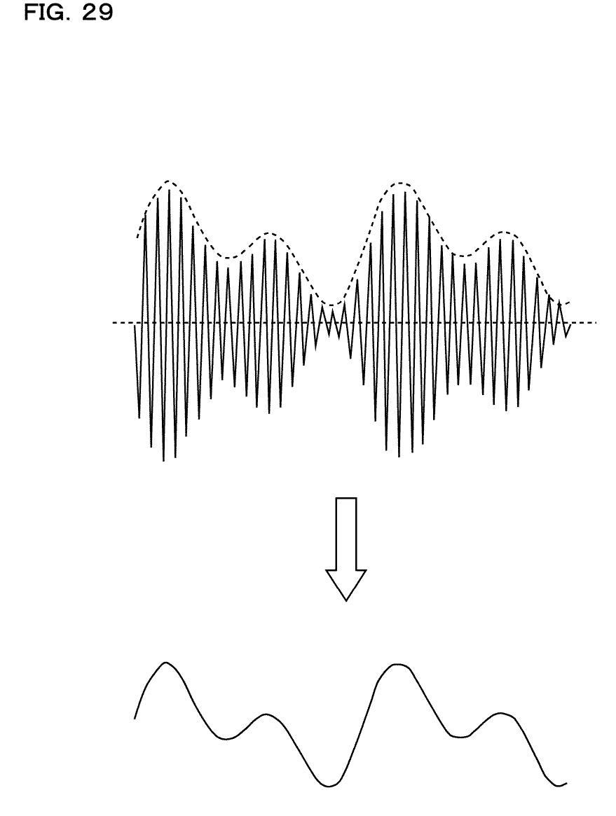

FIG. 29 is a schematic diagram illustrating the detection (demodulation) applied to a pulse wave signal in the detector circuit 310. The upper half of FIG. 28 shows the input signal to the detector circuit 310, and the lower half of FIG. 29 shows the output signal from the detector circuit 310. As shown in FIG. 20 referred to previously, the detector circuit 310 incorporated in the filter 12 is a so-called inverting half-wave rectification detector circuit; it extracts from a pulse-driven input signal, by extracting its envelope curve, an output signal and outputs this to the circuit at the succeeding stage.

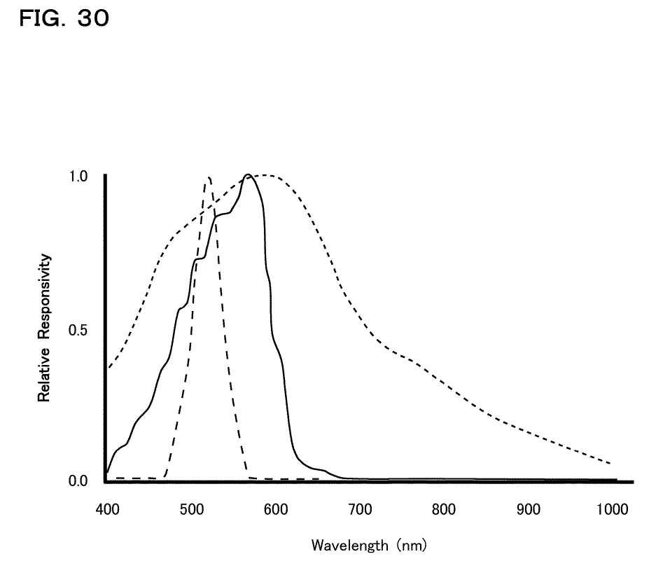

FIG. 30 is a chart showing the light-emission and -reception characteristics of the optical sensor 11. In FIG. 30, the horizontal axis indicates wavelength and the vertical axis indicates relative sensitivity. In the diagram, the solid line represents the wavelength characteristics (light-reception characteristics) of a new-type phototransistor, and the short-segment broken line represents the wavelength characteristics (light-reception characteristics) of an old-type phototransistor; the long-segment broken line represents the wavelength characteristics (light-emission characteristics) of an light-emitting diode. As shown in FIG. 30, in the new-type pulse wave sensor 1, the new-type phototransistor used as the light receiver is designed to have wavelength characteristics (light-reception characteristics) that match the wavelength characteristics (light-emission characteristics) of the light-emitting diode used as the light emitter. By optimizing the wavelength characteristics of the light emitter and the light receiver in this way, it is possible to cut down sensitivity in unnecessary bands, and thereby to reduce the influence of outside light (sunlight).

With the pulse wave sensor 1 adopting the combination of pulse lighting (FIGS. 20 and 27 to 29) and the wavelength characteristics optimization (FIG. 30) described above, it is possible to detect pulse waves with high accuracy not only indoors but also outdoors, where extraneous disturbing light is abundant.

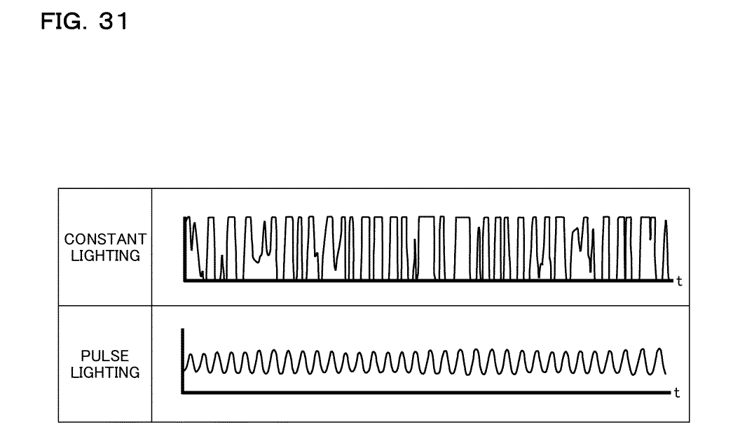

FIG. 31 is a table of comparison of measurement results between the new and old types as taken outdoors, with the test subject at rest (in a standing posture). The upper half of FIG. 31 shows results of pulse wave measurement outdoors (at 40000 lux) with the old-type pulse wave sensor 1, and the lower half of FIG. 31 shows results of pulse wave measurement outdoors (at 80000 lux) with the new-type pulse wave sensor 1. As shown in FIG. 31, with the new-type pulse wave sensor 1, the pulse wave signal is saturated under the influence of outside light (sunlight), making it impossible to measure pulse waves accurately. By contrast, with the new-type pulse wave sensor 1, it is possible to avoid saturation of the pulse wave signal and measure pulse waves accurately.

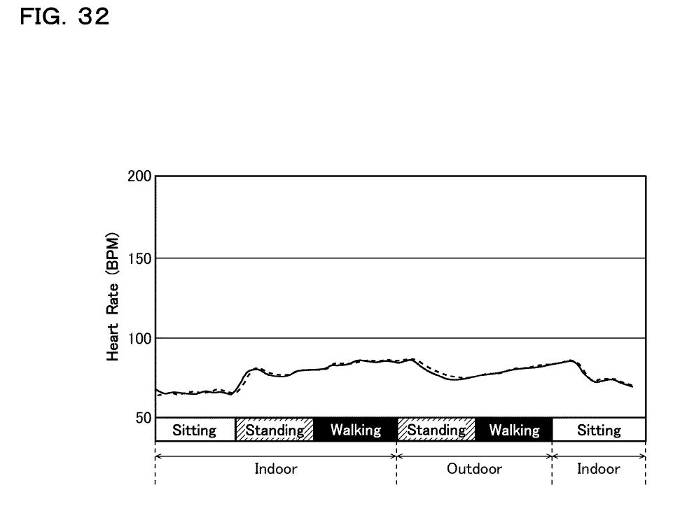

FIG. 32 is a chart showing results of pulse wave measurement outdoors with the new-type pulse wave sensor 1. In the chart, the solid line represents measurement results with the new-type pulse wave sensor 1, and the broken line represents, for comparison, measurement results with a chest-belt-worn heart rate meter (commercially available). As shown in FIG. 31, it was confirmed that, with the new-type pulse wave sensor 1, it is possible to obtain measurement results that correlate with those taken with a chest-belt-worn heart rate meter not only indoors but also outdoors (at 80000 lux), both with the test subject at rest (in a sitting or standing posture) and with the test subject walking.

The outdoor measurement technology (pulse lighting and wavelength characteristics optimization) described above can be applied not only to a wrist watch-type pulse wave sensor 1 but also to pulse wave sensors with any other structures (such as finger ring-type, eye mask-type, and an earplug-type).

### <Pulse Wave Measurement on an Ear>

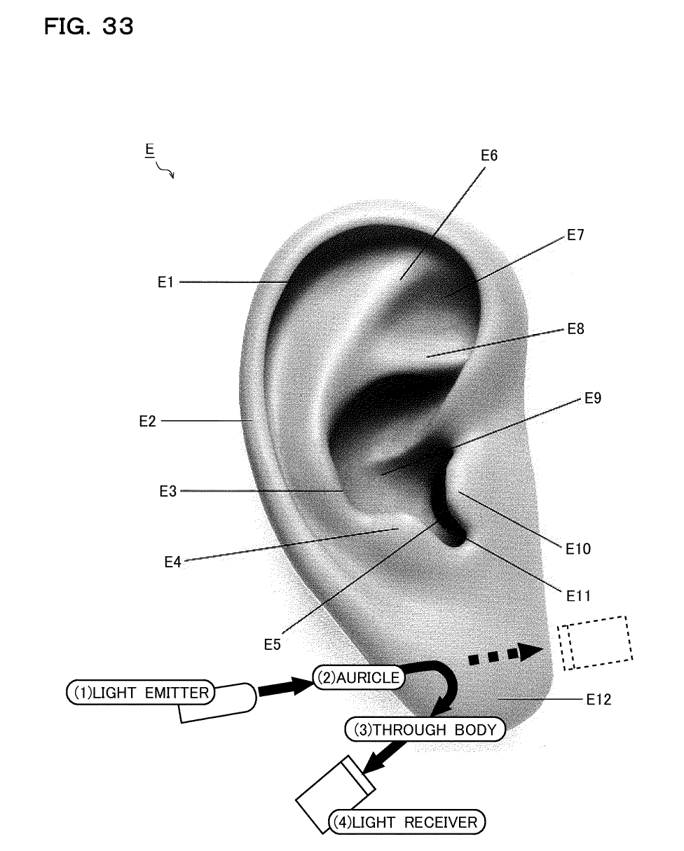

FIG. 33 is a schematic diagram illustrating the principle of pulse wave measurement on an ear. While the first and second embodiments described previously deal with configurations for pulse wave measurement chiefly on a wrist, a pulse wave sensor can be worn on any other part of the body than a wrist. Accordingly, the third embodiment of the present invention described below deals with a configuration for pulse wave measurement on an ear. When pulse wave measurement is performed on an ear, the pulse wave sensor (the light emitter and the light receiver) can be worn on any part of the outer ear E (e.g., scaphoid fossa E1, helix E2, antihelix E3, antitragus E4, external acoustic meatus (external ear canal) E5, superior antihelical crus E6, triangular fossa E7, inferior antihelical crus E8, concha auriculae E9, tragus E10, intertragic notch E11, or lobule E12).

### Pulse Wave Sensor

### Third Embodiment

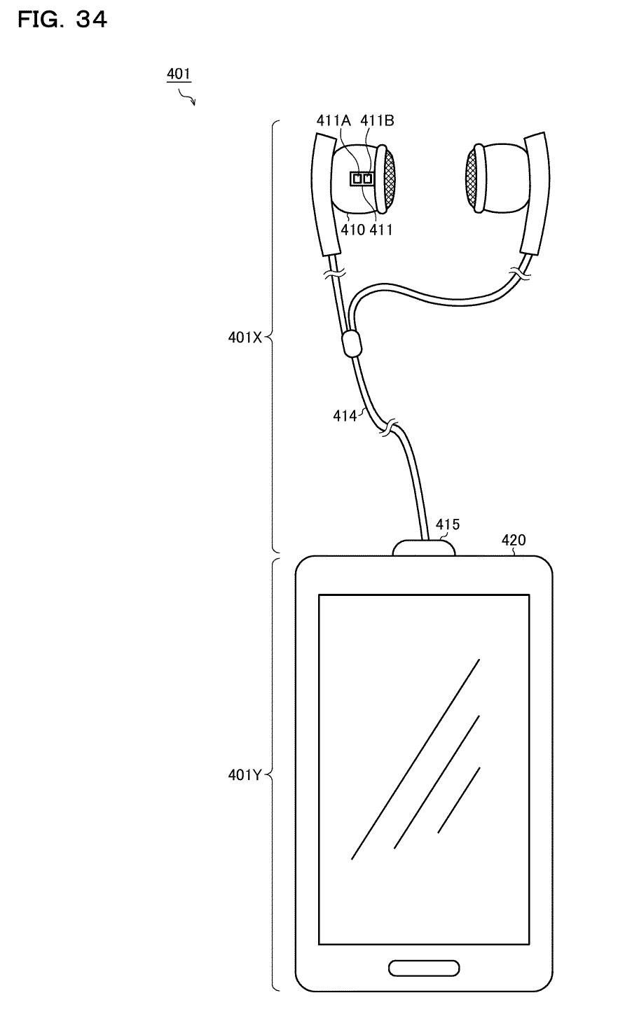

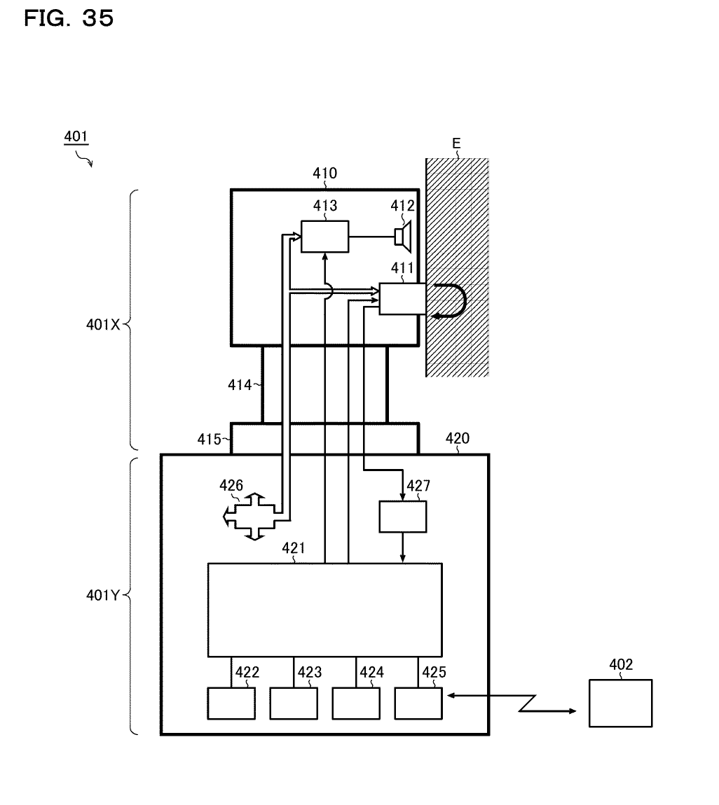

FIGS. 34 and 35 are an external view and a block diagram, respectively, of a pulse wave sensor according to a third embodiment of the present invention. The pulse wave sensor 401 of the third embodiment has an earphone (headphone) 401X and a main unit 401Y, and is offered as a portable audio player equipped with a pulse wave measurement function. Here, the concept of audio players covers not only devices dedicated to audio playback but also cellular telephone terminals, smartphones, portable game terminals, etc. equipped with an audio playback function.

The earphone 401X is of an inner ear type, meaning that it is, when in use, worn on a user's outer ear (in particular, auricle), and includes a housing 410, an optical sensor 411, a speaker 412, a driver 413, a cord 414, and a connector 415.

The housing 410 is a member which houses the optical sensor 411, the speaker 412, and the driver 413. The housing 410 has a shape that fits the pit surrounded by the tragus E10 and the antitragus E4 (the cymba conchae in the concha auriculae E9). The housing 410 may be of an open type or of a closed type.

The optical sensor 411 is arranged on a side face of the housing 410. Light from a light emitter 411A is shone on a predetermined part of the outer ear E, and the intensity of the light that returns after passing through the living body is detected with a light receiver 411B; thereby pulse wave data is acquired. Although FIG. 34 shows a configuration where a single optical sensor 411 is provided in one housing out of two for the right and left ears respectively, this is not meant to limit the number of optical sensors 411 provided; a plurality of optical sensors 411 may be provided in one of the housings 410, or a single optical sensor 411 or a plurality of optical sensors 411 may be provided in each of the housings 410. With a configuration where a single optical sensor 411 is provided, compared with a configuration where a plurality of optical sensors 411 are provided, priority can be given to power saving, cost reduction, etc. On the other hand, with a configuration where a plurality of optical sensors 411 are incorporated, it is possible to add up the outputs of the individual sensors to enhance the S/N ratio, or to selectively use the output of the sensor with the highest S/N ratio, thereby to enhance the detection accuracy of pulse waves. In a case where a plurality of optical sensors 411 are used selectively, by cutting off the supply of electric power to any unused optical sensor 411, it is possible to prevent a waste of electric power.

The pulse wave sensor 401 of the third embodiment adopts, instead of a configuration where the light emitter 411A and the light receiver 411B are arranged on opposite sides of the living body across it (a so-called transmission type configuration; see the broken-line arrow in FIG. 33), a configuration where the light emitter 411A and the light receiver 411B are both arranged on the same side of the living body 2 (a so-called reflection type configuration; see the solid-line arrows in FIG. 33). Moreover, the present inventors have, through experiments, confirmed that the latter configuration allows satisfactory pulse wave measurement when performed on the outer ear E. For specific structures of the optical sensor 411, the same structures as those of the optical sensor 11 in the first and second embodiments can be adopted, and therefore no overlapping description will be repeated.

The speaker 412 converts an audio signal (electrical signal) delivered from the main unit 401Y via the driver 413 into an acoustic wave and outputs it. The speaker 412 is typically driven dynamically, but may instead be driven in any other manner (such as magnetically, with a balanced armature, piezoelectrically, with a crystal, or electrostatically).

The driver 413 generates a drive signal for the speaker 412 based on the audio signal (electrical signal) delivered from the main unit 401Y.

The cord 414 is a member for electrically connecting between the housing 410 of the earphone 401X and the main unit 401Y. The cord 414 includes a signal transmission lead and a power supply lead.

The connector 415 is attached to one end of the cord 414, and is a member for disconnectably connecting the earphone 401X and the main unit 401Y together.

Instead of the cord 414 and the connector 415, wireless communication modules may be provided respectively in the housing 410 of the earphone 401X and in the main unit 401Y so that the two units are connected together wirelessly. In particular, when the main unit 401Y is given a watertight structure, from the perspective of completely eliminating external terminals from the main unit 401Y, it is preferable that the two units be connected together wirelessly. In that case, no electric power can be supplied from the main unit 401Y to the housing 410 of the earphone 401X, and accordingly a separate power supply needs to be provided in the housing 410 of the earphone 401X.

The main unit 401Y includes a housing 420, a controller 421, an operation panel 422, a display 423, a storage 424, a communicator 425, a power supply 426, and a filter 427. In a case where the main unit 401Y is a cellular telephone terminal equipped with an audio playback function, it further includes, in addition to those enumerated above, a microphone, a speaker, a telephone network interface, etc.

The housing 420 is a member which houses the controller 421, the operation panel 422, the display 423, the storage 424, the communicator 425, the power supply 426, and the filter 427. It is preferable that the housing 420 be given a watertight structure to prevent damage from immersion in water or the like.

The controller 421 controls the operation of the entire pulse wave sensor 401 in a centralized fashion not only to achieve both an audio playback function and a pulse wave measurement function individually but also to combine the two functions synergistically to produce an added value. As the controller 421, a CPU or the like can be suitably used. How the controller 421 specifically operates will be described in detail later.

The operation panel 422 is a human interface which accepts input operations (for turning the power on and off, controlling the sound volume, selecting music, and so forth) by the user (test subject). As the operation panel 422, various keys and buttons or a touch panel or the like can be suitably used.

The display 423 is provided on the obverse face of the main unit 401Y, and outputs display information (including information on audio playback and results of pulse wave measurement). As the display 423, a liquid crystal display panel or the like can be suitably used.

The storage 424 includes ROM (read-only memory) which stores, on a non-volatile basis, various programs read and executed by the controller 421; RAM (random-access memory) which is volatile and is used as an area for program execution by the controller 421; and integrated (or removable) flash memory in which the user (test subject) can store, on a non-volatile basis, arbitrary music data.

The storage 424 also includes RAM, EEPROM (electrically erasable programmable ROM), or the like which stores, on a volatile or non-volatile basis, pulse wave data (raw data, or processed data having undergone various kinds of processing) obtained by the controller 421. With a configuration including a means for storing pulse wave data as described above, it is possible, for example, to externally transmit the data accumulated in the storage 424 in bulk at predetermined time intervals; this permits the communicator 425 to be left in a stand-by state intermittently, and thus helps extend the battery-operated period of the pulse wave sensor 401.

The communicator 425 transmits to an external information terminal 402 (such as a data server or a personal computer) the measurement data of the pulse wave sensor 401 (raw data, processed data having undergone various kinds of processing, or the data stored in the storage 424) on a wireless or wired basis. In particular, with a configuration where the measurement data of the pulse wave sensor 401 are transmitted wirelessly to the information terminal 402, there is no need for wired connection between the pulse wave sensor 401 and the information terminal 402; this makes it possible, for example, to transmit measurement data on a real-time basis without restricting the user's (test subject's) activities. In particular, in a case where the main unit 401Y is given a watertight structure, from the perspective of completely eliminating external terminals from the main unit 401Y, it is preferable to adopt wireless communication as a method for external transmission of measurement data. In a case where measurement data are transmitted wirelessly to an information terminal 2 at a short distance (several meters to several tens of meters), the communicator 425 can suitably comprise a Bluetooth (a registered trademark) wireless communication module or the like. In a case where measurement data are transmitted to an information terminal 402 at a distant place over the Internet or the like, the communicator 425 can suitably comprise a wireless LAN (local area network) module or the like.

The power supply 426 includes a battery and a DC/DC converter; it converts an input voltage from the battery into a desired output voltage, and supplies it to different parts of the pulse wave sensor 401. A battery-operated pulse wave sensor 401 like this does not require connection by a cable for the supply of electric power from outside during pulse wave measurement, and thus allows pulse wave measurement without restricting the user's (test subject's) activities. As the battery just mentioned, it is preferable to use a secondary battery (such as a lithium-ion secondary battery or an electric double-layer capacitor), which allows repeated recharging. Using a secondary battery as the battery eliminates the need for troublesome battery replacement, and thus helps make the pulse wave sensor 1 more convenient to use. Power feeding from outside for battery charging can be achieved by contact power feeding, such as by use of a USB cable, or by non-contact power feeding, such as by electromagnetic induction, electric-field coupling, or magnetic resonance. In a case where the pulse wave sensor 401 is given a watertight structure, from the perspective of completely eliminating external terminals from the main unit 401Y, it is preferable to adopt non-contact power feeding for power feeding from outside.

The filter 427 applies filtering and amplification to the output signal of the optical sensor 411 (the detection signal of the light receiver) and delivers the result to the controller 421. A filter may be provided in the housing 410 of the earphone 401X, but considering that noise is likely to be superimposed on the signal being transmitted from the housing 410 of the earphone 401X via the cord 414 to the main unit 401Y, it is preferable to provide the filter 427 in the main unit 401Y. For specific circuit configurations of the filter 427, the same configurations as those of the filter in the first and second embodiments can be adopted, and therefore no overlapping description will be repeated.

As described above, the pulse wave sensor 401 of the third embodiment has a housing 410 which is worn on the outer ear E, and an optical sensor 411 which is provided in the housing 410 and which acquires pulse wave data by irradiating the outer ear E with light from a light emitter 411A and detecting with a light receiver 411B the light that returns after passing through the living body.

With this configuration, unless the user (test subject) intentionally removes the pulse wave sensor 401 from the outer ear E, the pulse wave sensor 401 is unlikely to drop off the outer ear E during pulse wave measurement. Thus, it is possible to measure pulse waves without restricting the user's (test subject's) activities.

In particular, the outer ear E is a part of the body subject to less motion than a finger or an arm; thus, the output signal of the optical sensor 411 is less likely to be affected by body motion noise, and this permits pulse wave measurement with high accuracy.

Moreover, with the pulse wave sensor 401 that incorporates the optical sensor 411 in the earphone 401X which is worn on the outer ear E chiefly for the purpose of listening to sound, the user (test subject) can wear, on a day-to-day basis, the pulse wave sensor 401 as a portable audio player equipped with a pulse wave measurement function. This helps alleviate the awkwardness from wearing the pulse wave sensor 401, making it possible to widen the scope of use and to develop a new group of users.

Moreover, the controller 421, which controls the operation of the entire pulse wave sensor 401 in a centralized fashion, not only achieves both an audio playback function and a pulse wave measurement function individually but also, with a view to combining the two functions synergistically to produce an added value, is furnished with a function of controlling the output operation of the speaker 412 according to pulse wave data.

Specifically, the controller 421 applies various kinds of signal processing to the output signal of the filter 427, thereby acquires various kinds of information on pulse waves (fluctuations in pulse waves, heart rate, variations in heart rate, acceleration pulse waves, etc.), and feeds results of their analysis back to audio playback operation.

For example, based on results of analysis of pulse wave data, the controller 421 determines the user's (test subject's) physical and mental condition, sleep condition, etc.; then based on results of such determination, the controller 421 automatically adjusts the sound volume, selects music, turns the power on or off, and so forth. With this configuration, it is possible to realize audio playback operation that cannot be realized with a dedicated portable audio player.

Although FIGS. 34 and 35 show a configuration where the earphone 401X and the main unit 401Y are provided as separate units, this is not meant to limit the configuration of the pulse wave sensor 401; the earphone 401X and the main unit 401Y may be configured integrally. In that case, the cord 414 and the connector 415 are no longer necessary.

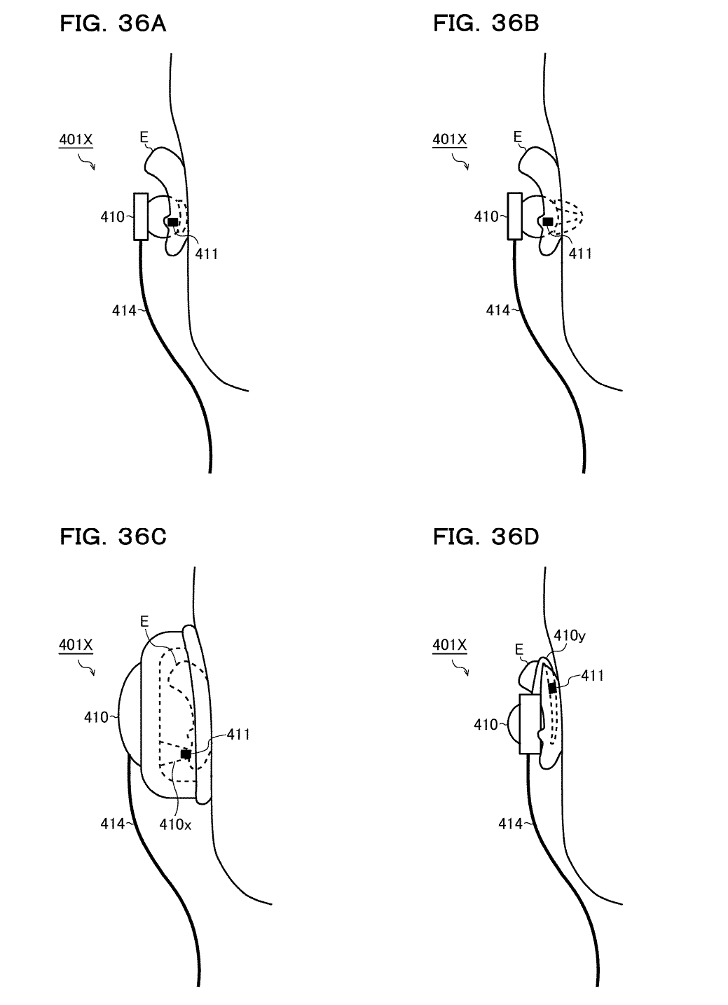

Also as to how the earphone 401X is shaped and how it is worn on the outer ear E, many variations are possible as shown in FIGS. 36A to 36D. FIGS. 36A to 36D are front views schematically showing a first to a fourth design, respectively, of the earphone 401X and how the earphone 401X of each design is worn on the outer ear E.

For example, the earphone 401X of the first design (FIG. 36A) is, like the previously described one shown in FIG. 34, of an inner ear type, and its housing 410 has a shape (e.g., spherical or cylindrical) that fits the pit surrounded by the tragus E10 and the antitragus E4 (the cymba conchae in the concha auriculae E9). In the earphone 401X of the first design, the optical sensor 411 rests in (abuts on the inside of) the pit.

The earphone 401X of the second design (FIG. 36B) is of an earplug type (canal type) in which, during its use, an earpiece formed of silicone or urethane foam is inserted deep into the external ear canal E5, and its housing 410, like that in the first design (FIG. 36A), has a shape that fits the pit surrounded by the tragus E10 and the antitragus E4 (the cymba conchae in the concha auriculae E9). In the earphone 401X of the second design, as in the first design, the optical sensor 411 rests in (abuts on the inside of) the pit.

The earphone 401X of the third design is of a headphone type which is provided with a housing 410 so shaped as to cover the entire auricle E. A right and a left housing 410 (for the right and left ears respectively) are so configured as to be held across the test subject's head with the help of a headband worn over the head or a neckband worn around a rear part of the neck (neither is illustrated). In the earphone 401X of the third design, the housing 410 has a protruding member 410x which holds the optical sensor 411 on the face (inner side face) of the housing 410 facing the auricle E. The protruding member 410x protrudes toward the auricle E, and the optical sensor 411 is mounted, for example, at its tip. Accordingly, in the earphone 401X of the third design, the optical sensor 411 abuts on a part (e.g., lobule E12) of the outer ear that faces the tip of the protruding member 410x. In the earphone 401X of the third design, the housing 410 covering the entire auricle E also functions as a light-shielding member for covering the optical sensor 411. With this configuration, it is possible to perform pulse wave measurement stably without being affected by outside light.

The earphone 401X of the fourth design (FIG. 36D) is of a hooked-on-ear type which has a clip member 410y, which is hooked on the auricle E. The clip member 410y holds the optical sensor 11 in a part thereof abutting on the auricle E. Accordingly, in the earphone 401X of the fourth design, the optical sensor 411 abuts on a part of the auricle E at or around the back of the superior antihelical crus E6, triangular fossa E7, inferior antihelical crus E8, or concha auriculae E9.

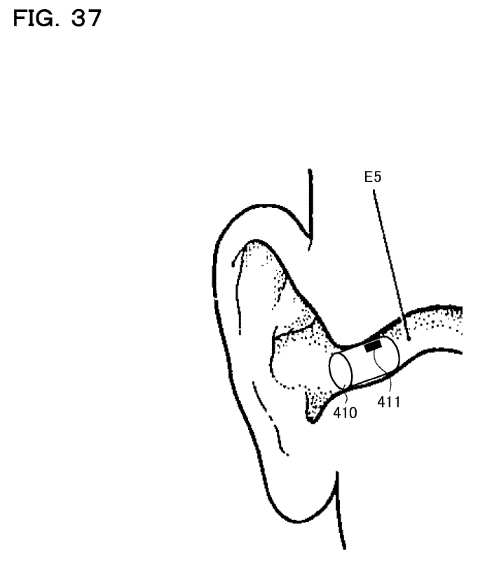

Although the above description deals with, as examples, configurations where the optical sensor 411 is provided in an earphone or a headphone, this is not meant to limit the configuration of the pulse wave sensor 401; for example, as in a modified example shown in FIG. 37, a configuration is possible where an optical sensor 411 is held in a housing 410 having an earplug structure so that pulse waves are measured inside the external ear canal E5. In that case, the housing 410 is inserted deep into the external ear canal E5 so as to stop it, and the optical sensor 411 abuts on the inner wall face of the external ear canal E5. With a pulse wave sensor 401 having an earplug structure like this, the function of the earplug itself can be exploited to relax the test subject, and thus the test subject can go through pulse wave measurement without feeling excessive stress. With this feature, a pulse wave sensor 401 having an earplug structure can be suitably used as a sleep soundness sensor (a sensor for evaluating the test subject's sleep condition based on pulse waves information).

Irrespective of which of the configurations described above is adopted, it is preferable that the light receiver 411B be arranged closer to the external ear canal E5 (or deeper in the external ear canal E5) than the light emitter 411A is. With this configuration, outside light is less likely to leak into the light receiver 411B, and this helps enhance the detection accuracy of pulse wave data.

### <Application to Hearing Aids>

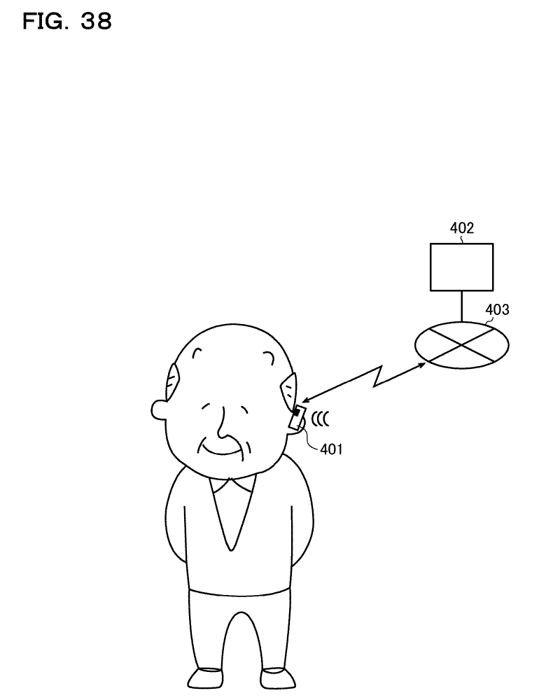

FIG. 38 is a system diagram showing an example of application to a hearing aid. The pulse wave sensor 401 in FIG. 38 is offered as a hearing aid equipped with a pulse wave measurement function. As a specific configuration of the pulse wave sensor 401, one similar to that shown in FIG. 35 can be adopted, but here such components (such as a sound collecting microphone) as are needed to function not as a portable audio player but as a hearing aid need to be incorporated.

Moreover, an information terminal 402 as a destination of transmission of pulse wave data and results of their analysis (such as well-being information) is supposed to be installed at a distant place. Accordingly, in an application to a hearing aid, it is preferable that the pulse wave sensor 401 be provided with a communicator (such as a wireless LAN module) for establishing connection with the information terminal 402 (such as a data server at a medical facility or a personal computer owned by a test subject's family living at a distant place) over a network 403.

Users (test subjects) who need a hearing aid include those who require health monitoring and well-being check from a distant place. However, it is not always easy for aged people to properly wear and maintain a plurality of electronic devices (here, a hearing aid and a pulse wave sensor) individually.

By contrast, the pulse wave sensor 401 offered as a hearing aid equipped with a pulse wave measurement function is itself a hearing aid for the user (test subject), and thus leaves the user unconscious of pulse wave measurement. This helps alleviate the burden of wearing and maintaining it. Moreover, by monitoring the pulse wave data and the results of their analysis transmitted from the pulse wave sensor 401 on the information terminal 402 at a distant place, it is possible to promptly deal with an abnormality in the user's (test subject's) health condition.

Naturally, the configuration for measuring pulse waves on the outer ear E can be applied to pulse wave sensors that are not equipped with an additional function such as an audio playback function or a hearing aid function.

### <Sleep Sensor>

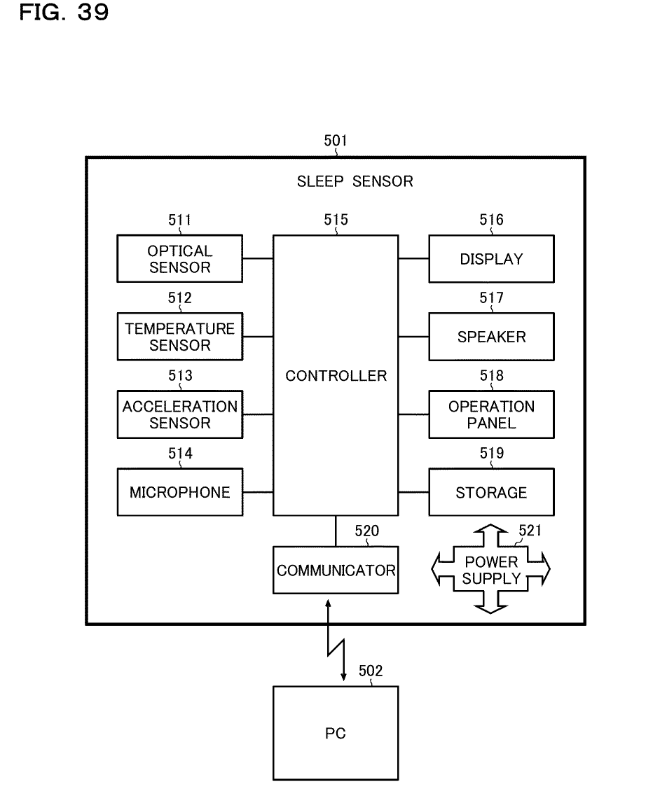

FIG. 39 is a block diagram showing a configuration example of a sleep sensor (an example of application as a physical condition management system). The sleep sensor 501 of this configuration example has an optical sensor 511, a temperature sensor 512, an acceleration sensor 513, a microphone 514, a controller 515, a display 516, a speaker 517, an operation panel 518, a storage 519, a communicator 520, and a power supply 521.

The optical sensor 511 acquires measurement data on the test subject's pulse waves and blood oxygen saturation level by irradiating the test subject's living body with light and detecting the intensity of the light returning after passing through the living body. The optical sensor 511 can be configured like those in the first to third embodiments described previously, and therefore no overlapping description will be repeated.

The temperature sensor 512 acquires measurement data on the test subject's body temperature and body surface temperature.

The acceleration sensor 513 acquires measurement data on the test subject's body motion.

The microphone 514 acquires measurement data on the sound and voice produced by the test subject and the ambient sound around the test subject.

The controller 515 controls the operation of the entire sleep sensor 501 in a centralized fashion. As the controller 515, a CPU or the like can be suitably used.

The display 516 outputs images (including characters and the like) according to the test subject's sleep condition. As the display 516, a liquid crystal display panel or the like can be suitably used.

The speaker 517 outputs sound (including alerting sounds and the like) according to the test subject's sleep condition.

The operation panel 518 is a human interface which accepts input operations (such as for turning the power on and off) by the test subject. As the operation panel 518, various keys and buttons, a touch panel, or the like can be suitably used.

The storage 519 includes ROM which stores, on a non-volatile basis, various programs read and executed by the controller 515; and RAM which is volatile and is used as an area for program execution by the controller 515.

The storage 519 further includes RAM, EEPROM, or the like which stores, on a volatile or non-volatile basis, measurement data obtained by the pulse wave sensor 1 (raw data, or processed data having undergone various kinds of processing). With a configuration including a means for storing pulse wave data as described above, it is possible, for example, to externally transmit the data accumulated in the storage 519 at in bulk predetermined time intervals; this permits the communicator 520 to be left in a stand-by state intermittently, and thus helps extend the battery-operated period of the sleep sensor 501.

The communicator 520 transmits to an external information terminal 502 (such as a data server or a personal computer) the measurement data obtained by the sleep sensor 501 (raw data, processed data having undergone various kinds of processing, or the data stored in the storage 519) on a wireless or wired basis. In particular, with a configuration where the measurement data acquired by the sleep sensor 501 are transmitted wirelessly to the information terminal 502, there is no need for wired connection between the pulse wave sensor 501 and the information terminal 502; this makes it possible, for example, to transmit measurement data on a real-time basis without restricting the test subject's activities. In particular, in a case where the sleep sensor 501 is given a watertight structure, from the perspective of completely eliminating external terminals from the sleep sensor 501, it is preferable to adopt wireless communication as a method for external transmission of measurement data. In a case where measurement data are transmitted wirelessly to an information terminal 502 at a short distance (several meters to several tens of meters), the communicator 502 can suitably comprise a Bluetooth (a registered trademark) wireless communication module or the like. In a case where measurement data is transmitted to an information terminal 502 at a distant place over the Internet or the like, the communicator 520 can suitably comprise a wireless LAN (local area network) module or the like.

The power supply 521 includes a battery and a DC/DC converter; it converts an input voltage from the battery into a desired output voltage, and supplies it to different parts of the sleep sensor 501. A battery-operated sleep sensor 501 like this does not require connection by a cable for the supply of electric power from outside during sleep condition monitoring, and thus allows sleep condition monitoring without restricting the user's (test subject's) activities. As the battery just mentioned, it is preferable to use a secondary battery (such as a lithium-ion secondary battery or an electric double-layer capacitor), which allows repeated recharging. Using a secondary battery as the battery eliminates the need for troublesome battery replacement, and thus helps make the pulse wave sensor 1 more convenient to use. Power feeding from outside for battery charging can be achieved by contact power feeding, such as by use of a USB cable, or by non-contact power feeding, such as by electromagnetic induction, electric-field coupling, or magnetic resonance. In a case where the sleep sensor 501 is given a watertight structure, from the perspective of completely eliminating external terminals from the sleep sensor 501, it is preferable to adopt non-contact power feeding for power feeding from outside.

By building a physical condition management system including a sleep sensor 501 which is worn by a test subject and an information terminal 502 which analyzes and takes a log of measurement data acquired by the sleep sensor 501 as described above, it is possible, without giving the sleep sensor 501 itself unnecessarily high functionality, to monitor the test subject's day-to-day sleep condition and perform proper physical condition management. Moreover, by acquiring data from a large number of test subjects and collecting them on the information terminal 502, it is possible to perform a statistical analysis or the like.

For the reason given above, it is preferable to leave a detailed analysis of measurement data acquired by the sleep sensor 501 to an external information terminal 502; nevertheless, it is very useful to furnish the controller 515 with a function of analyzing the test subject's sleep condition based on measurement data acquired by the sleep sensor 501 and accordingly driving the display 516 and the speaker 517.

For example, the controller 515 can be configured to determine whether the test subject is in REM or non-REM sleep based on measurement data (heart rate, variations in heart rate, etc.) on the test subject's pulse waves and accordingly drive the display 516 and the speaker 517. For example, by outputting wake-up music or environmental sound (such as songs of birds and murmuring of a stream) from the speaker 517 when the test subject is found in REM sleep, it is possible to provide the test subject with comfortable awakening. The controller 515 can instead be configured to determine the depth of the test subject's sleep based on measurement data on the test subject's pulse waves and accordingly drive the display 516 and the speaker 517.

The controller 515 can be configured to determine whether the test subject has an apnea syndrome (the quality of sleep) based on measurement data on the test subject's blood oxygen saturation level and accordingly drive the display 516 and the speaker 517. For example, by sounding an alarm from the speaker 517 when the test subject has an attack of apnea, it is possible to forcibly wake up the test subject or notify a person nearby of the abnormality in the test subject.

The controller 515 can be configured to determine the depth of the test subject's sleep based on measurement data on the test subject's body temperature or body surface temperature to accordingly drive the display 516 and the speaker 517. For example, by outputting wake-up music or environmental sound from the speaker 517 when the test subject comes to have shallower sleep and a raised body temperature, it is possible to provide the test subject with comfortable awakening.

The controller 515 can be configured to determine the depth of the test subject's sleep based on measurement data on the test subject's body motion to accordingly drive the display 516 and the speaker 517. For example, by outputting wake-up music or environmental sound from the speaker 517 when the test subject comes to have shallower sleep and exhibit more body motion, by outputting wake-up music or environmental sound from the speaker 517 when the test subject comes to have shallower sleep and a raised body temperature, it is possible to provide the test subject with comfortable awakening.

The controller 515 can be configured to determine the test subject's condition (snoring and teeth grinding (bruxism)) based on measurement data on the sound and voice produced by the test subject and the ambient sound around the test subject to accordingly drive the display 516 and the speaker 517. For example, by sounding an alarm when the test subject is snoring hard, it is possible to forcibly wake up the test subject or notify a person nearby of the abnormality in the test subject.

Although the examples described above deal with configurations where the display 516 and the speaker 517 incorporated in the sleep sensor 501 are driven and controlled according to the test subject's sleep condition, this is not meant to limit the target of driving and control by the controller 515; it is also conceivable to remote-control a home electric appliance provided outside the sleep sensor 501.

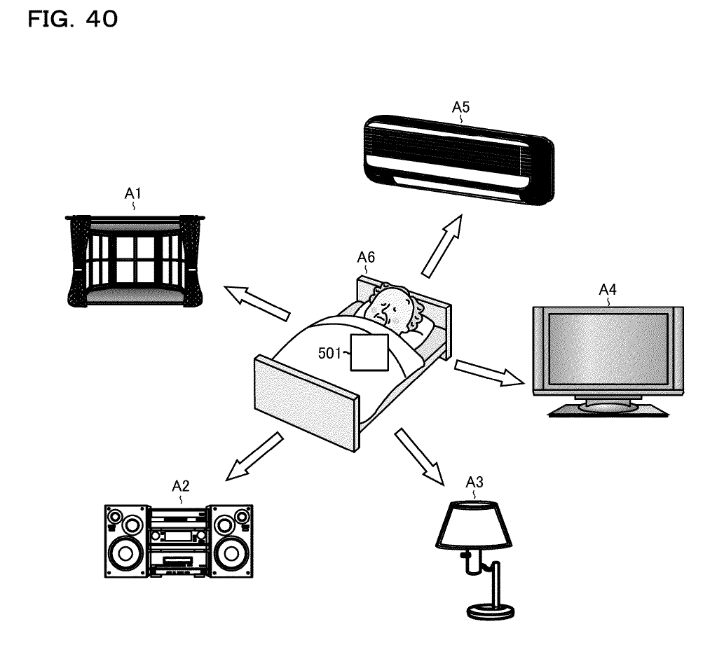

FIG. 40 is a schematic diagram showing a configuration example of a home electric appliance control system that employs the sleep sensor 501. In the home electric appliance control system of this configuration example, an electrically-operated curtain A1, an audio appliance A2, a lighting appliance A3, a television A4, an air conditioner A5, and a bed appliance (such as an electrically-operated bed or a pneumatic mattress) A6 are controlled according to the test subject's sleep condition as determined by use of the sleep sensor 501.

With the home electric appliance control system of this configuration example, for example, as the test subject wakes up, the electrically-operated curtain A1 is drawn open, the audio appliance A2 plays wake-up music, the lighting appliance A3 is lighted, the television A4 selects a news channel, the air conditioner A5 conditions the bed room at a comfortable temperature, and the bed appliance A6 adjusts itself into a setting that allows the test subject to rise with ease (by adjusting the reclining angle of the electrically-operated bed or adjusting the pressure in the pneumatic mattress).

Thus, with the home electric appliance control system of this configuration example, various home electric appliances A1 to A6 can be operated in coordination with the sleep sensor 501 to provide the test subject with comfortable awakening.

Although FIG. 40 shows, as an example, a configuration where the home electric appliances A1 to A6 are controlled directly from the sleep sensor 501, this is not meant to limit the configuration of a home electric appliance control system; for example, in a case where there is provided an information terminal 502 (see FIG. 39) which analyzes various kinds of measurement data acquired by the sleep sensor 501, the home electric appliances A1 to A6 may be controlled from the information terminal 502.

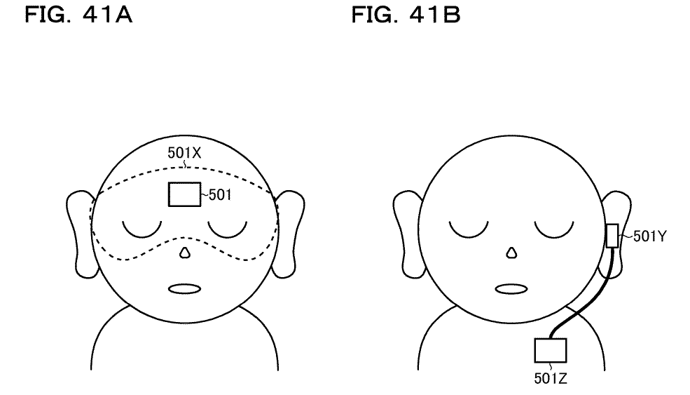

FIG. 41A is a schematic diagram showing a first example of how the sleep sensor 501 (of a type worn on the forehead) is worn. In FIG. 41A, a body of the sleep sensor 501 is arranged in a central part (where it abuts on the test subject's glabella) of an eye mask-type housing 501X (see the broken line in the figure). With the sleep sensor 501 arranged in this way on the glabella where blood capillaries concentrate, it is possible to stably measure pulse waves and blood oxygen saturation level with the optical sensor 511, and this helps enhance the accuracy of sleep condition monitoring. Moreover, the eye mask-type housing 501X also functions as a light-shielding member which covers the sleep sensor 501. With this configuration, the optical sensor 511 is less likely to be influenced by outer light, and this makes it possible to stably perform sleep condition monitoring. Moreover, the eye mask-type housing 501X has its inherent function of relaxing the test subject, and thus the test subject can go through sleep condition monitoring without feeling excessive stress during.

FIG. 41B is a schematic diagram showing a second example of how the sleep sensor 501 (of a type worn on an ear) is worn. In FIG. 41B, a sensor unit 501Y which is worn on the test subject's outer ear and a main unit 501Z which is worn on the test subject's collar or chest are provided separately, as discrete units. The sensor unit 501Y houses various sensors 511 to 514, and the main unit 501Z houses other components 515 to 521. This configuration helps make compact the sensor unit 501Y which is worn on the test subject's outer ear, preventing the test subject from feeling awkward. In particular, the outer ear is a part of the body subject to less motion than a finger or an arm; thus, the output signal of the optical sensor 511 is less likely to be effected by body motion noise, and this permits high-accuracy measurement of pulse waves and blood oxygen saturation level. As to the design of the sensor unit 501Y, it is possible to adopt any of the designs of common earphones (inner ear-type, canal type, clip type, etc.), or to adopt an earplug-type design for insertion into the external ear canal (see FIGS. 36A to 36D and 37).

### <Studies on Output Wavelength>

Experiments were conducted with a so-called reflection-type pulse wave sensor to study its behavior with its light emitter operated to emit at output wavelengths of λ1 (infrared, 940 nm), λ2 (green, 630 nm), and λ3 (blue, 468 nm), at each of output strengths (drive current levels) of 1 mA, 5 mA, and 10 mA. The results revealed that, in a visible region of the spectrum, at wavelengths of about 600 nm or less, the coefficient of oxygenated hemoglobin HbO₂ absorption is so high, and thus the peak strength of the measured pulse waves is so high, that the waveform of pulse waves is comparatively easy to acquire.

Incidentally, in pulse oximeters, which are used to detect oxygen saturation level in arterial blood, the light emitter is typically operated at output wavelengths in a near-infrared region of the spectrum (around 700 nm) at which the difference is largest between the coefficient of oxygenated hemoglobin HbO₂ absorption (a solid line) and the coefficient of deoxygenated hemoglobin Hb absorption (a broken line). However, from the viewpoint of use as a pulse wave sensor (in particular, a so-called reflection-type pulse wave sensor), it can be said that it is preferable that the light emitter be operated at an output wavelength in a visible region of the spectrum, at wavelengths of 600 nm or less.

However, in a case where a single optical sensor is used to detect both pulse waves and blood oxygen saturation level, it may be operated at a wavelength in a near-infrared region of the spectrum as conventionally practiced.

### Pulse Wave Sensor

### Fourth Embodiment

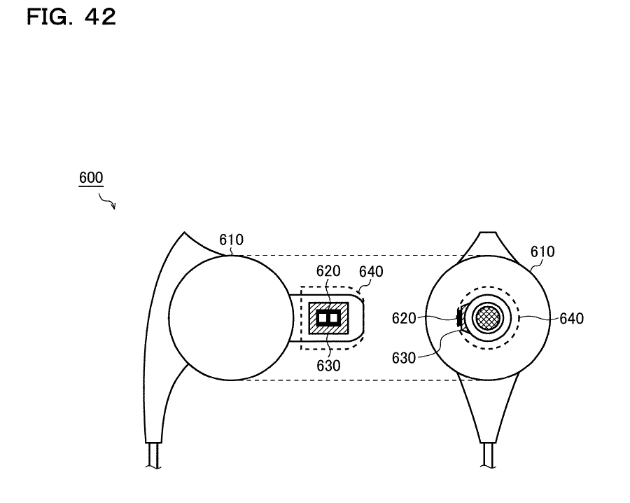

FIG. 42 is a block diagram showing a pulse wave sensor according to a fourth embodiment of the present invention. The pulse wave sensor 600 of the fourth embodiment is, like that of the third embodiment, of an ear-worn type (e.g., a canal type as shown in FIG. 36B), and includes a housing 610, an optical sensor 620, a damping member 630, and an close-contact member 640.

In particular, with a view to achieving more accurate pulse wave measurement in activities and outdoors, the pulse wave sensor 600 adopts a novel body motion noise suppression structure and a novel outside light suppression structure which have been developed for application to an ear-worn type. Accordingly, the following description is focused on the novel structures adopted in the pulse wave sensor 600, and with the understanding that, otherwise, whichever of the configurations and operations described thus far are suitable can be applied here as well, no overlapping description will be repeated.

The housing 610 is a member which is worn on the outer ear E (see FIG. 33). The housing 610 is connected, on a wired or a wireless basis, to a main unit (not illustrated) which analyzes and records pulse wave data. In a case where the pulse wave sensor 600 is offered as an earphone equipped with a pulse wave measurement function, a sound outputting means (a speaker, a driver, etc.) is incorporated in the housing 610 as necessary.

The optical sensor 620 is provided in the housing 610 (e.g., in a protruding portion which is inserted into the external ear canal E5); it acquires pulse wave data by irradiating a predetermined part of the outer ear E (e.g., the inner wall of the external ear canal E5) with light from a light emitter and detecting with a light receiver the intensity of the light returning after passing through the living body. To reduce the influence of outside light, it is preferable that the light receiver be arranged closer to the external ear canal E5 than the light emitter is.

The damping member 630 is a highly vibration-absorbent (flexible, or elastic) member which is provided between the housing 610 and the optical sensor 620. As the damping member 630, urethane sponge can be suitably used. This, however, is not meant to limit the material of the damping member 630; a gel material or a rubber material may instead be used. Providing the damping member 630 helps alleviate propagation of vibration from the housing 610 to the optical sensor 620; this helps reduce variation in the optical distance between the optical sensor 620 and the outer ear E, and thus helps reduce body motion noise. It is thus possible to perform stable pulse wave measurement not only with the test subject at rest but also with the test subject in activity.

In particular, to enhance the effect of vibration propagation suppression, it is preferable that the damping member 630 be provided between the housing 610 and the optical sensor 620 with the damping member 630 compressed in its height direction. With consideration given both to accuracy of pulse wave measurement (specific measurement results will be presented later) and to ease of wearing on the outer ear E, it is preferable that the damping member 630 be designed to have, when uncompressed, a height of 2.5±1.0 cm.

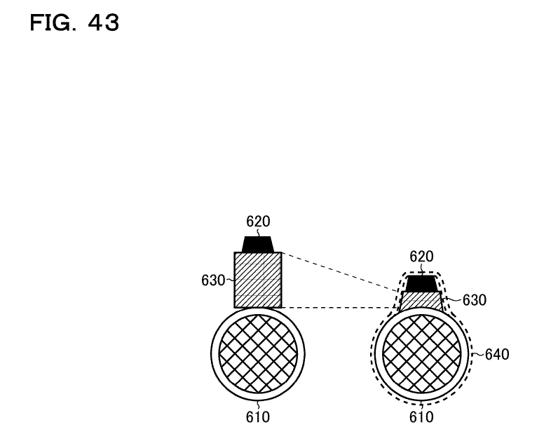

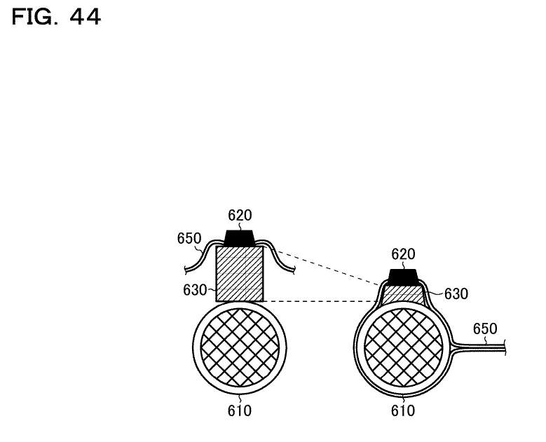

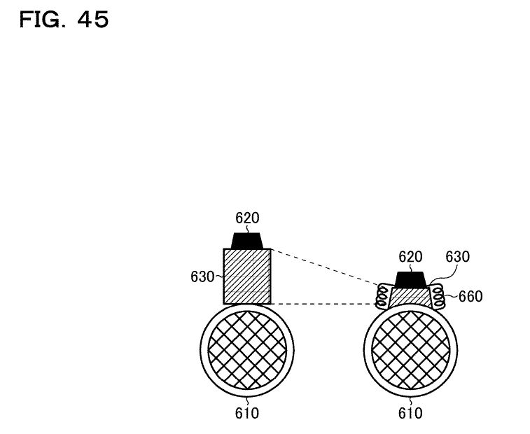

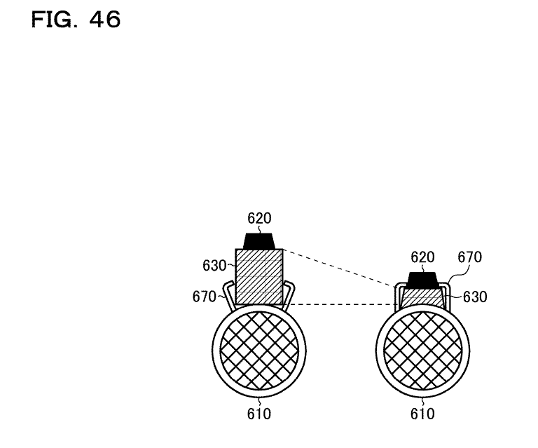

As a method for compressing the damping member 630, it is possible to use, for example, a method in which the damping member 630 is compressed by the contracting force of the close-contact member 640 which covers the optical sensor 620 (see FIG. 43); a method in which the damping member 630 is compressed by a binding force of leads 650 laid from opposite ends of the optical sensor 620 (see FIG. 44); a method in which the damping member 630 is compressed by the contracting force of an elastic member 660 (e.g., a spring) that couples the housing 610 and the optical sensor 620 together (see FIG. 45); a method in which the damping member 630 is compressed by the locking force of protruding members 670 that couple the housing 610 and the optical sensor 620 together (see FIG. 46); or any combination of the methods just enumerated.

The close-contact member 640 is a member for enhancing the ease of wearing on the outer ear E (a so-called earpiece). As the close-contact member 640, a material that provides close contact with the living body, such as silicone rubber, can be suitably used. In particular, in the pulse wave sensor 600, the close-contact member 640 transmits light at the light emission wavelength (meaning that it transmits the light exiting from and entering the optical sensor 620), and the optical sensor 620 is arranged at a position where it is covered by the close-contact member 640. This configuration helps enhance the closeness of contact between the optical sensor 620 and the outer ear E; this helps reduce the optical distance between the optical sensor 620 and the outer ear E, and thus helps reduce body motion noise. It is thus possible to perform stable pulse wave measurement not only with the test subject at rest but also with the test subject in activity.

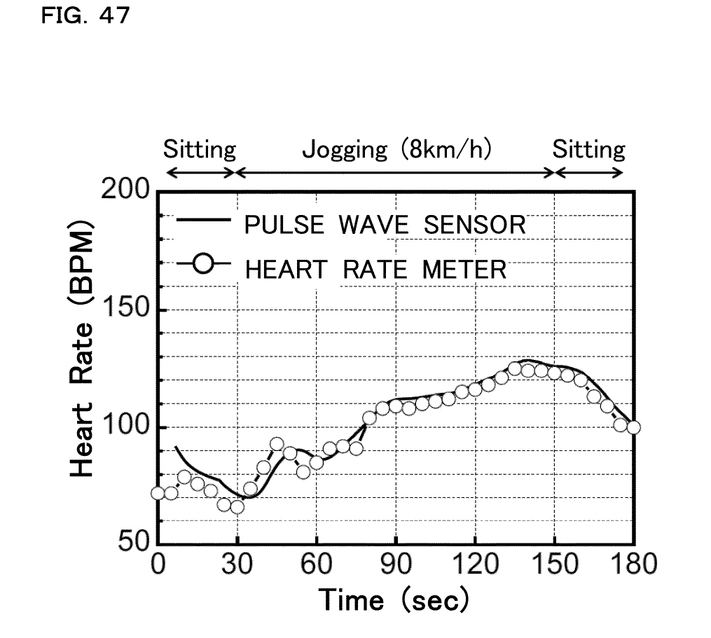

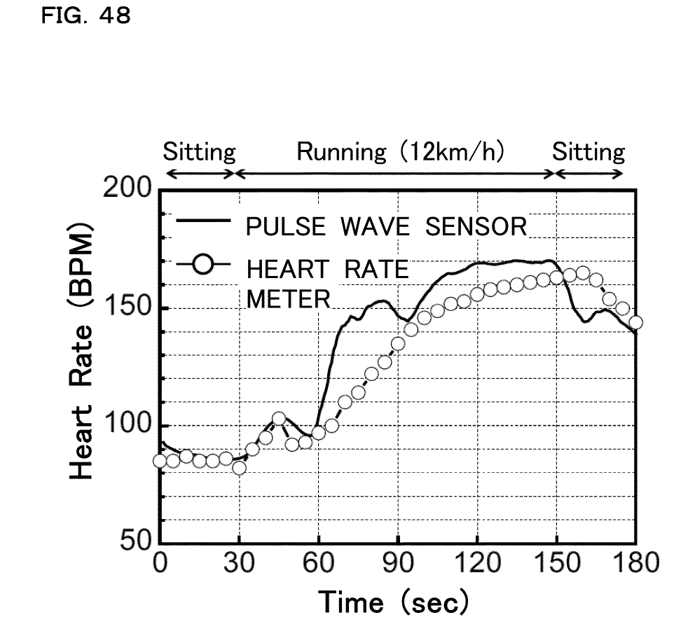

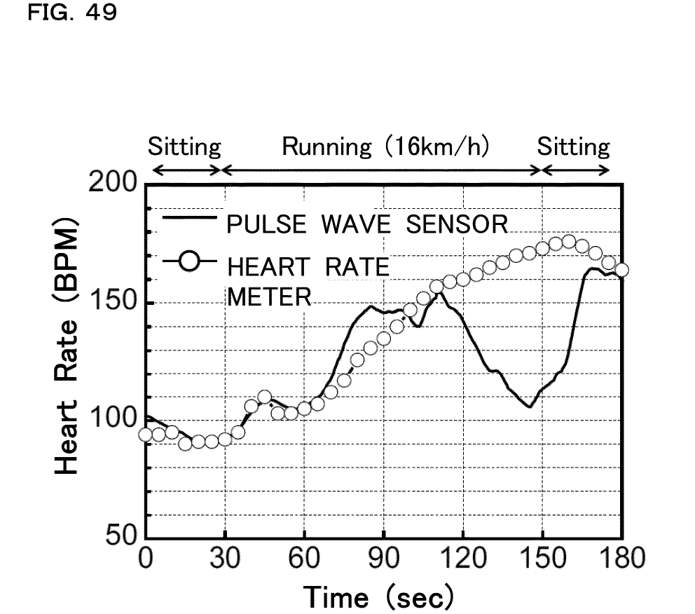

FIGS. 47 to 49 show results of pulse wave measurement done at different traveling speeds (8 km/h, 12 km/h, and 16 km/h respectively) under a first condition: with no earpiece (close-contact member 640) and with no sponge (damping member 630).

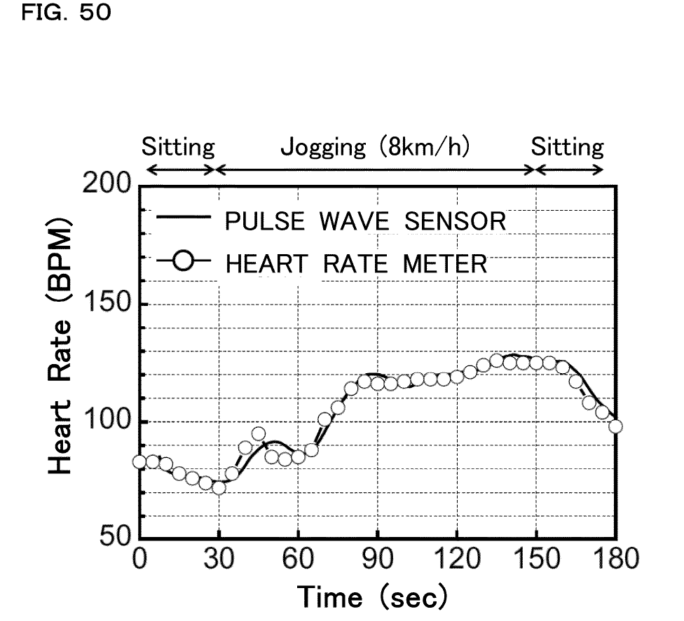

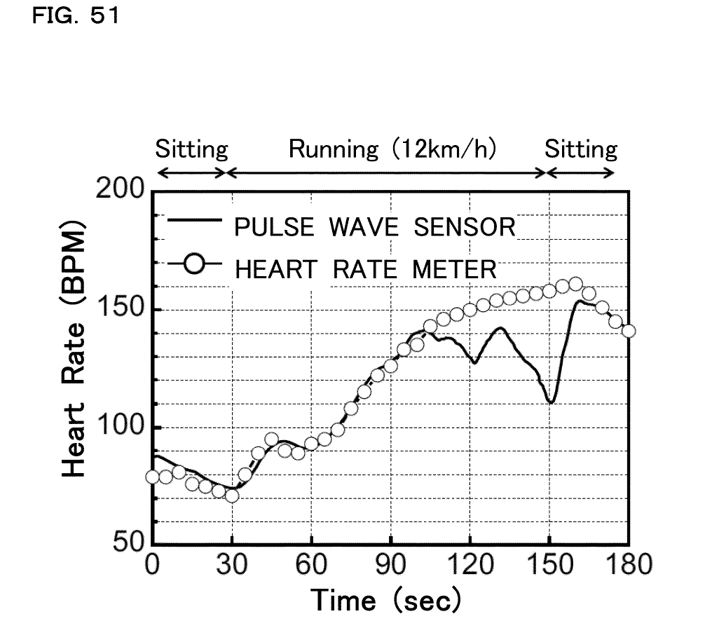

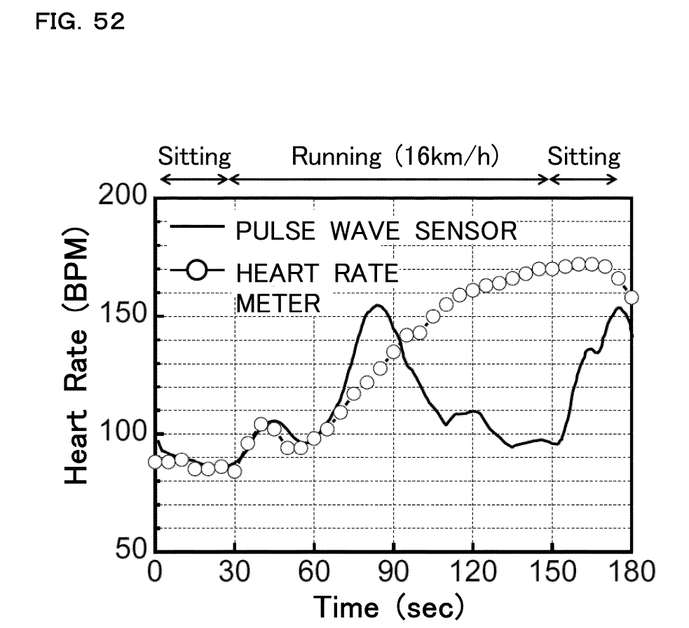

FIGS. 50 to 52 show results of pulse wave measurement done at different traveling speeds (8 km/h, 12 km/h, and 16 km/h respectively) under a second condition: with an earpiece (close-contact member 640) but with no sponge (damping member 630).

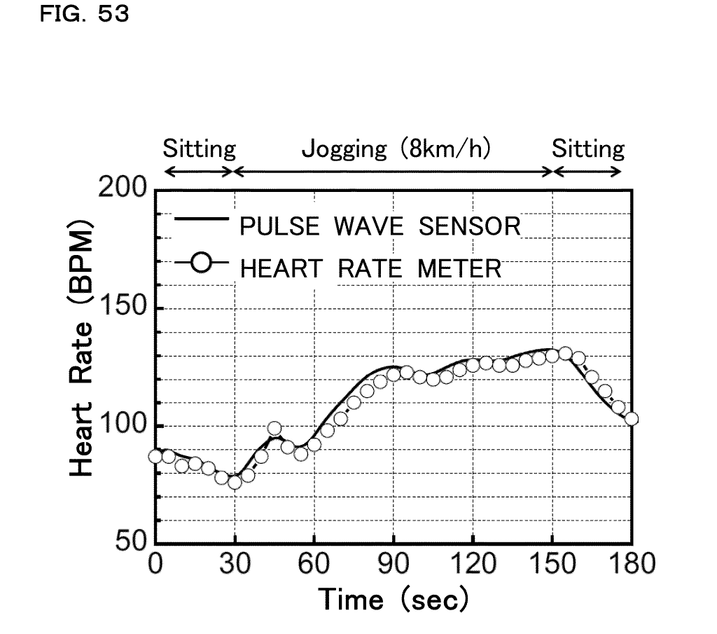

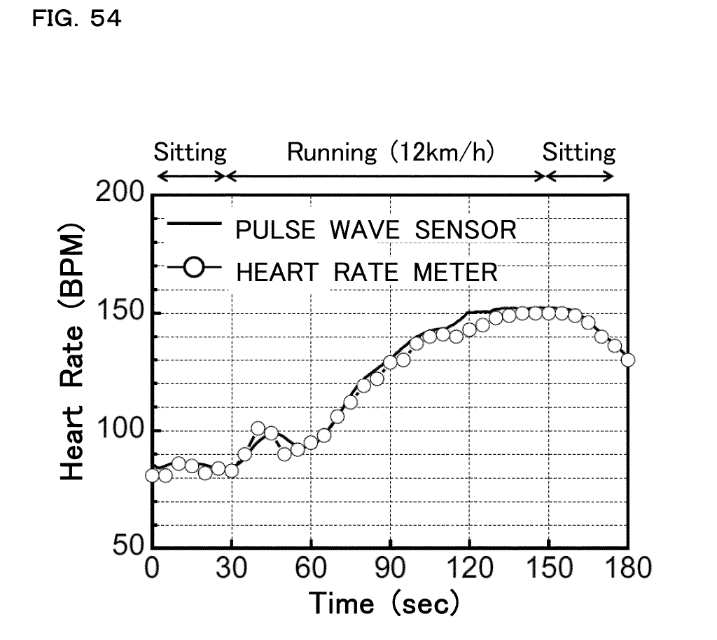

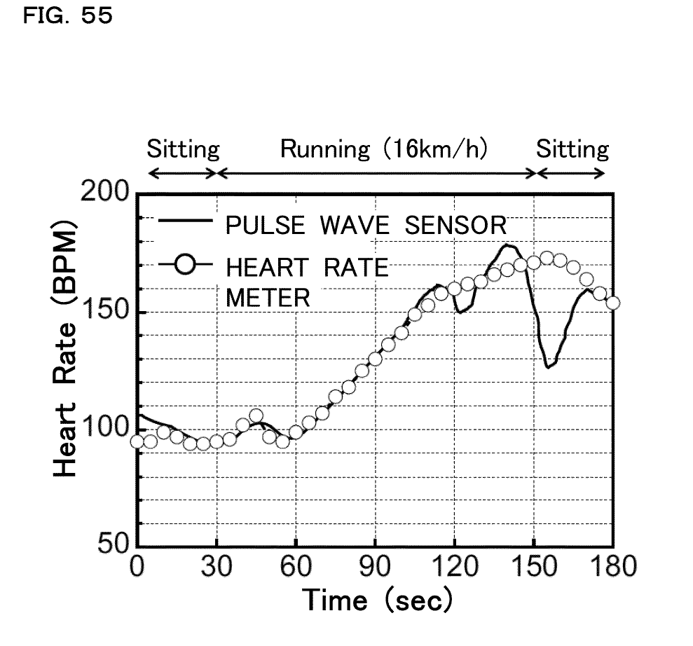

FIGS. 53 to 55 show results of pulse wave measurement done at different traveling speeds (8 km/h, 12 km/h, and 16 km/h respectively) under a third condition: with an earpiece (close-contact member 640) and with a 1 cm thick sponge (damping member 630).

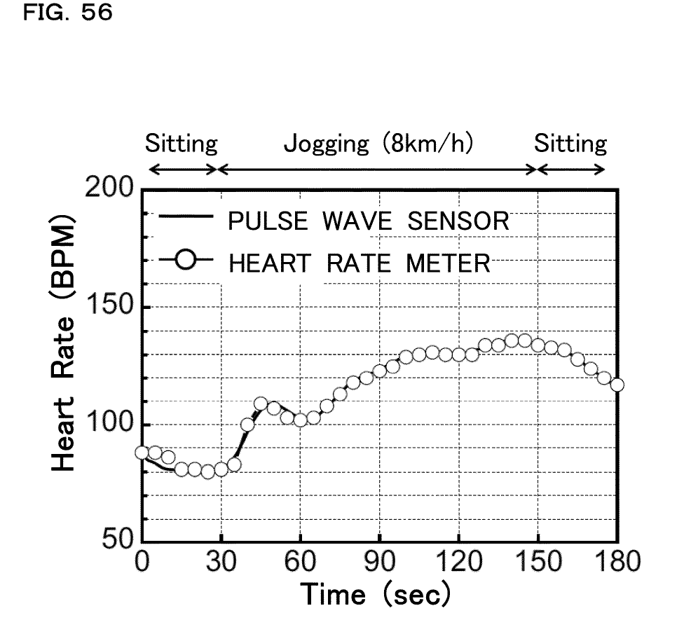

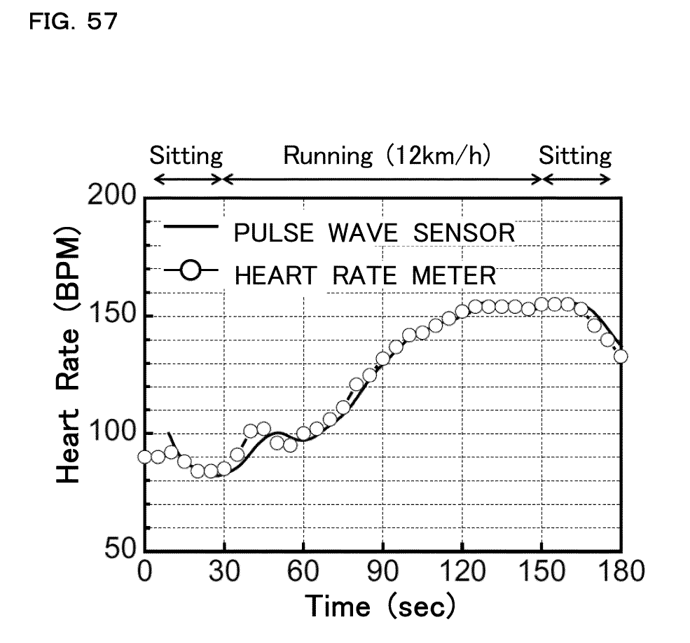

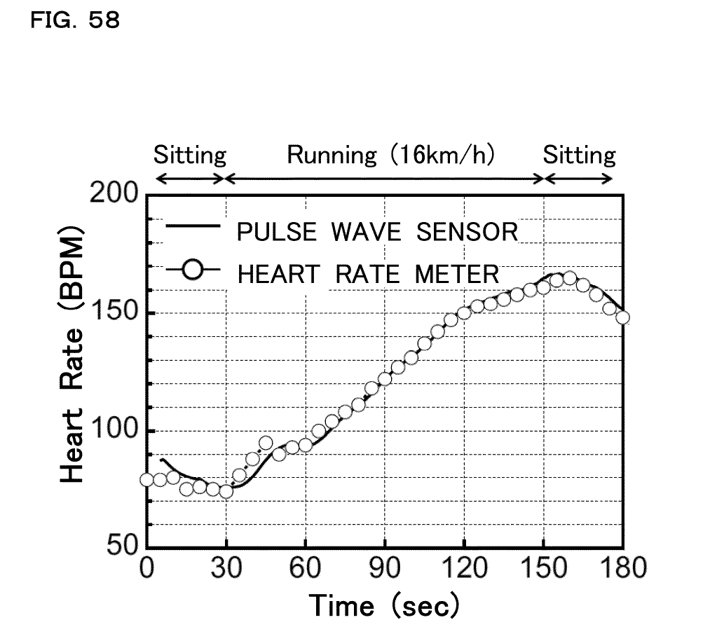

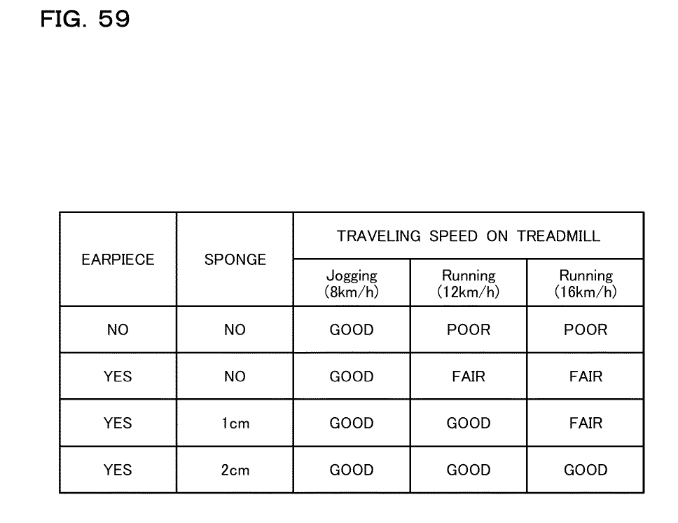

FIGS. 56 to 58 show results of pulse wave measurement done at different traveling speeds (8 km/h, 12 km/h, and 16 km/h respectively) under a fourth condition: with an earpiece (close-contact member 640) and with a 2 cm thick sponge (damping member 630).

In all the charts, a solid line represents measurement results with the pulse wave sensor 600, and circles represent, for comparison, measurement results with a chest belt-worn heart rate meter (commercially available). All the activities (running) involved in pulse wave measurement were performed indoors, on a treadmill.

As shown in FIGS. 47 to 49, under the first condition, stable pulse wave measurement was possible with the test subject at rest (in a sitting posture) and with the test subject jogging (8 km/h), but not with the test subject running (12 km/h and 16 km/h).

As shown in FIGS. 50 to 52, under the second condition, stable pulse wave measurement was possible with the test subject at rest (in a sitting posture) and with the test subject jogging (8 km/h), but not with the test subject running (12 km/h and 16 km/h), though a slight improvement was observed compared with the first condition.

As shown in FIGS. 53 to 55, under the third condition, stable pulse wave measurement was possible not only with the test subject at rest (in a sitting posture) and with the test subject jogging (8 km/h) but also with the test subject running (12 km/h). However, with the test subject running at a higher speed (16 km/h), pulse wave measurement was slightly less stable.

As shown in FIGS. 56 to 58, under the fourth condition, stable pulse wave measurement was possible not only with the test subject at rest (in a sitting posture) and with the test subject jogging (8 km/h) but with the test subject running (12 km/h and 16 km/h).

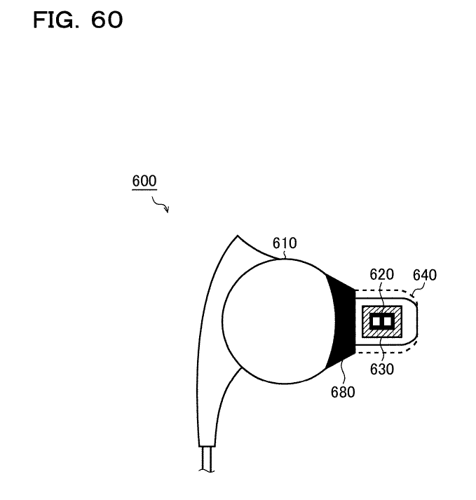

FIG. 60 shows a table that summarizes the results of the above-mentioned measurements done under different conditions. The results shown there verify that providing the damping member 630 and the close-contact member 640 enables stable pulse wave measurement not only with the test subject at rest but also with the test subject in activity.

FIG. 60 is an exterior view of a first modified example of the fourth embodiment. The pulse wave sensor 600 of the first modified example further has a light-shielding member 680 (e.g., a black sheet) for preventing entry of outside light into the optical sensor 620. With this configuration, it is possible to prevent outside light from leaking into the optical sensor 620, and thus to perform high-accuracy detection of pulse waves not only indoors but also outdoors, where extraneous disturbing light is abundant.

As shown in FIG. 60, it is preferable that the light-shielding member 680 be arranged outward of the optical sensor 620 (on the far side with respect to the external ear canal E5) so as to stop the open end of the close-contact member 640. It is also effective to surround the optical sensor 620 with a black sheet. However, to prevent the light-shielding member 680 from acting as a vibration propagation path from the housing 610 to the optical sensor 620, it is preferable that the housing 610 and the light-shielding member 680 not be fastened together.

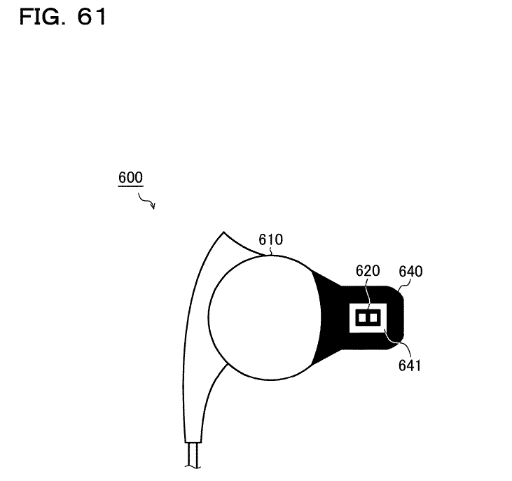

FIG. 61 is an exterior view of a second modified example of the fourth embodiment. The pulse wave sensor 600 of the second modified example is a further development of the first modified example described previously. Here, the close-contact member 640 transmits light only in a part thereof, serving as a measurement window 641, that covers the optical sensor 620, and is made back elsewhere to function as a light-shielding member. With this configuration, the close-contact member 640 functions as a light-shielding member as well, and this helps reduce the number of components.

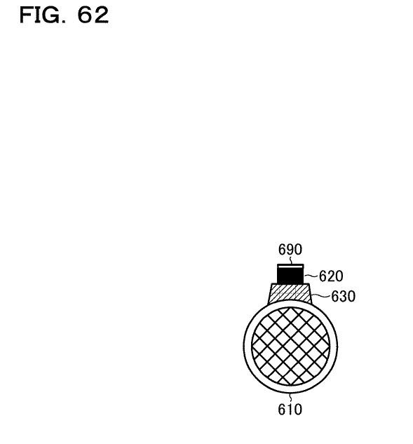

FIG. 62 is an exterior view of a third modified example of the fourth embodiment. The pulse wave sensor 600 of the third modified example adopts, instead of a configuration where a close-contact member 640 provided as an earpiece covers the optical sensor 620, a configuration where a close-contact member 690 for enhancing the closeness of contact between the optical sensor 620 and the outer ear E is provided on the surface of an optical sensor 620. With this configuration, for example, even in a case where the optical sensor 620 is provided at a position difficult to cover with an earpiece, it is possible to enhance the closeness of contact between the optical sensor 620 and the outer ear E and thereby to reduce body motion noise. The close-contact member 690 can be formed by various methods such as by coating with silicone resin or by affixing a silicone resin sheet.

It is particularly preferable to provide all of the damping member 630, the light-shielding member 680, and the close-contact member 690 described above in combination. Needless to say, however, depending on the use of the pulse wave sensor 600, each of them may be implemented individually, or part of them may be implemented in combination.

### <Recapitulation>

To follow is a recapitulation of various aspects of the present invention disclosed herein.

### [First Aspect of the Invention]

Of the various aspects of the present invention disclosed herein, according to a first aspect, a pulse wave sensor can be configured as one including an optical sensor which acquires pulse wave data by irradiating a living body with light from a light emitter and detecting with a light receiver the intensity of the light that has passed through the living body, wherein the optical sensor includes a box-shaped case; and a light-shielding wall which divides the case into a first region, where the light emitter is mounted, and a second region, where the light receiver is mounted (Configuration 1-1).

The pulse wave sensor of Configuration 1-1 can be so configured that, between the height H1 of the light-shielding wall and the height H2 of the light emitter, the relationship H1>H2 holds (Configuration 1-2).

The pulse wave sensor of Configuration 1-2 can be so configured that the offset distance ΔH (=H1−H2) calculated by subtracting the height H2 of the light emitter from the height H1 of the light-shielding wall is in the range of 0 mm<ΔH<2 mm (Configuration 1-3).

The pulse wave sensor of Configuration 1-2 or 1-3 can be so configured that, between the height H2 of the light emitter and the height H3 of the light receiver, the relationship H2>H3 holds (Configuration 1-4).

The pulse wave sensor of any of Configurations 1-1 to 1-4 can be so configured that the chip-to-chip distance W1 between the light emitter and the light receiver is the range of 0.2 mm≦W1≦0.8 mm (Configuration 1-5).

The pulse wave sensor of any of Configurations 1-1 to 1-5 can be so configured that the optical sensor has a condenser lens over the light emitter (Configuration 1-6).

The pulse wave sensor of any of Configurations 1-1 to 1-6 can be so configured that the first region is covered by a first lid member having a first opening smaller than the light emission region of the light emitter (Configuration 1-7).

The pulse wave sensor of any of Configurations 1-1 to 1-7 can be so configured that the second region is covered by a second lid member having a second opening larger than the light reception region of the light receiver (Configuration 1-8).

The pulse wave sensor of any of Configurations 1-1 to 1-8 can be so configured that at least one of the light emitter and light receiver has a color filter that selectively transmits a predetermined wavelength component (Configuration 1-9).

The pulse wave sensor of any of Configurations 1-1 to 1-9 can be so configured that the light emitter and the light receiver each include a substrate, a light-emitting chip or a light-receiving chip mounted on the substrate, and a seal which seals the light-emitting or -receiving chip (Configuration 1-10).

The pulse wave sensor of any of Configurations 1-1 to 1-10 can be so configured that the case is buried in a body which holds the optical sensor, in such a way that the case protrudes from the body (Configuration 1-11).

The pulse wave sensor of any of Configurations 1-1 to 1-11 can be so configured that the output wavelength of the light emitter is in a visible region of the spectrum, about 600 nm or less (Configuration 1-12).

### [Second Aspect of the Invention]

Of the different aspects of the present invention disclosed herein, according to a second aspect, a pulse wave sensor can be configured as one having an optical sensor which acquires pulse wave data by irradiating a living body with light from a light emitter and detecting with a light receiver the intensity of the light that has passed through the living body; and a body which holds the optical sensor, wherein the body is a member which, when the pulse wave sensor is worn on the living body, is given a pressing force toward the living body, and the optical sensor is arranged on the surface of the body, near the force application point where the pressing force toward the living body is strongest (Configuration 2-1).

The pulse wave sensor of Configuration 2-1 can be so configured that a belt is connected to opposite ends of the body, and the optical sensor is arranged at a distance of 10 mm or less from where the belt is connected to the body (Configuration 2-2).

The pulse wave sensor of Configuration 2-1 can be so configured that a spring hinge is connected to a first end of the body and a second end of the body is left as an open end, with the optical sensor arranged at a distance of 10 mm or less from the second end of the body (Configuration 2-3).

The pulse wave sensor of any of Configurations 2-1 to 2-3 can be so configured that the optical sensor comprises a plurality of optical sensors which are arranged on the surface of the body, in a region near the force application point where the pressing force toward the living body is strongest (Configuration 2-4).

The pulse wave sensor of any of Configurations 2-1 to 2-4 can be so configured that the output wavelength of the light emitter is in a visible region of the spectrum, about 600 nm or less (Configuration 2-5).

### [Third Aspect of the Invention]

Of the different aspects of the present invention disclosed herein, according to a third aspect, a pulse wave sensor can be configured as one having an optical sensor which acquires pulse wave data by irradiating a living body with light from a light emitter and detecting with a light receiver the intensity of the light that has passed through the living body; and a filter which applies filtering to the output signal of the optical sensor, wherein the filter includes a high-pass filter circuit which eliminates a low-frequency component superimposed on the output signal of the optical sensor; a voltage follower circuit which delivers the output signal of the high-pass filter circuit to the succeeding stage; a low-pass filter circuit which eliminates a high-frequency component superimposed on the output signal of the voltage follower circuit; a first amplifier circuit which amplifies the output signal of the low-pass filter circuit; a band-pass filter circuit which eliminates a low-frequency component and a high-frequency component superimposed on the output signal of the first amplifier circuit; and a second amplifier circuit which amplifies the output signal of the band-pass filter circuit (Configuration 3-1).

The pulse wave sensor of Configuration 3-1 can be so configured that the high-pass filter circuit is a first-order CR high-pass filter circuit having a cut-off frequency of 0.66 Hz (Configuration 3-2).

The pulse wave sensor of Configuration 3-1 or 3-2 can be so configured that the low-pass filter circuit is a second-order CR low-pass filter circuit having a cut-off frequency of 0.26 Hz (Configuration 3-3).

The pulse wave sensor of any of Configurations 3-1 to 3-3 can be so configured that the band-pass filter circuit is a sixth-order band-pass filter circuit having a pass band of 0.80 Hz to 2.95 Hz (Configuration 3-4).

The pulse wave sensor of any of Configurations 3-1 to 3-4 can be so configured that the filter includes an intermediate voltage generator circuit which divides a supply voltage to produce an intermediate voltage, and the high-pass filter circuit, the low-pass filter circuit, the first amplifier circuit, the band-pass filter circuit, and the second amplifier circuit all operate relative to the intermediate voltage as a reference voltage (Configuration 3-5).

The pulse wave sensor of any of Configurations 3-1 to 3-5 can be so configured that the output wavelength of the light emitter is in a visible region of the spectrum, about 600 nm or less (Configuration 3-6).

[Fourth Aspect of the Invention]

Of the different aspects of the present invention disclosed herein, according to a fourth aspect, a pulse wave sensor can be configured as one having a housing which is worn on an outer ear; and an optical sensor which is provided in the housing and which acquires pulse wave data by irradiating the outer ear with light from a light emitter and detecting with a light receiver the intensity of the light returning after passing through the living body (Configuration 4-1).

The pulse wave sensor of Configuration 4-1 can be so configured that the housing has a speaker (Configuration 4-2).

The pulse wave sensor of Configuration 4-2 can be so configured as to have a controller which controls output operation of the speaker according to the pulse wave data (Configuration 4-3).

The pulse wave sensor of any of Configurations 4-1 to 4-3 can be so configured as to have a communicator which transmits the pulse wave data to an information terminal (Configuration 4-4).

The pulse wave sensor of any of Configurations 4-1 to 4-4 can be so configured that the housing has a shape that fits the pit surrounded by the tragus and the antitragus (Configuration 4-5).

The pulse wave sensor of Configuration 4-5 can be so configured that the light receiver is arranged closer to the external ear canal than the light emitter is (Configuration 4-6).

The pulse wave sensor of any of Configurations 4-1 to 4-4 can be so configured that the housing has a shape that covers the auricle (Configuration 4-7).

The pulse wave sensor of Configuration 4-7 can be so configured that the housing has, on a face thereof facing the auricle, a protruding member which holds the optical sensor (Configuration 4-8).

The pulse wave sensor of any of Configurations 4-1 to 4-4 can be so configured that the housing has a clip member which is hooked on the auricle (Configuration 4-9).

The pulse wave sensor of Configuration 4-9 can be so configured that the clip member holds, in a part thereof abutting on the auricle, the optical sensor (Configuration 4-10).

The pulse wave sensor of Configuration 4-1 can be so configured that the housing has an earplug structure for measuring pulse waves inside the external ear canal (Configuration 4-11).

The pulse wave sensor of any of Configurations 4-1 to 4-11 can be so configured that the optical sensor has a box-shaped case; and a light-shielding wall which divides the case into a first region, where the light emitter is mounted, and a second region, where the light receiver is mounted (Configuration 4-12).

The pulse wave sensor of Configuration 4-12 can be so configured that, among the height H1 of the light-shielding wall, the height H2 of the light emitter, and the height H3 of the light receiver, the relationship H1>H2>H3 holds (Configuration 4-13).

The pulse wave sensor of Configuration 4-13 can be so configured that the case is buried in the housing such that the former protrudes from the latter (Configuration 4-14).

The pulse wave sensor of any of Configurations 4-1 to 4-14 can be so configured that the optical sensor has, between itself and the housing, a damping member (Configuration 4-16).

The pulse wave sensor of any of Configurations 4-1 to 4-15 can be so configured that that the output wavelength of the light emitter is in a visible region of the spectrum, about 600 nm or less (Configuration 4-16).

[Fifth Aspect of the Invention]

Of the different aspects of the present invention disclosed herein, according to a fifth aspect, a sleep sensor can be configured as one having an optical sensor which acquires measurement data on a test subject's pulse waves, or measurement data on a test subject's pulse waves and blood oxygen saturation level; a temperature sensor which acquires measurement data on the test subject's body temperature or body surface temperature; an acceleration sensor which acquires measurement data on the test subject's body motion; a microphone which acquires measurement data on the sound and voice produced by the test subject or on the ambient sound; a controller which controls the operation of the entire sleep sensor in a centralized fashion; a display which outputs images; a speaker which outputs sound; an operation panel which accepts input operations; a storage which stores the different measurement data; a communicator which transmits the different measurement data to an information terminal which analyzes the test subject's sleep condition; and a power supply which feeds electric power to the different parts of the sleep sensor (Configuration 5-1).

The sleep sensor of Configuration 5-1 can be so configured that the controller is furnished with a function of analyzing the test subject's sleep condition by analyzing the different measurement data (Configuration 5-2).

The sleep sensor of Configuration 5-2 can be so configured that the controller determines, based on the measurement data on the test subject's pulse waves, at least whether the test subject is in REM sleep or in non-REM sleep or the depth of the test subject's sleep, and accordingly drives the display, the speaker, or an external home electric appliance (Configuration 5-3).

The sleep sensor of Configuration 5-2 or 5-3 can be so configured that the controller determines, based on the measurement data on the test subject's blood oxygen saturation level, whether the test subject has an apnea syndrome, and accordingly drives the display, the speaker, or an external home electric appliance (Configuration 5-4).

The sleep sensor of any of Configurations 5-2 to 5-4 can be so configured that the controller determines, based on the measurement data on the test subject's body temperature or body surface temperature, the depth of the test subject's sleep, and accordingly drives the display, the speaker, or an external home electric appliance (Configuration 5-5).

The sleep sensor of any of Configurations 5-2 to 5-5 can be so configured that the controller determines, base on the measurement data on the test subject's body motion, the depth of the test subject's sleep, and accordingly drives the display, the speaker, or an external home electric appliance (Configuration 5-6).

The sleep sensor of any of Configurations 5-2 to 5-6 can be so configured that the controller determines, based on the measurement data on the sound and voice produced by the test subject or on the ambient sound, the test subject's condition, and accordingly drives the display, the speaker, or an external home electric appliance (Configuration 5-7).

The sleep sensor of any of Configurations 5-1 to 5-7 can be so configured that the optical sensor acquires measurement data on the test subject's pulse waves, or measurement data on the test subject's pulse waves and blood oxygen saturation level, by irradiating the test subject's living body with light from a light emitter and detecting with a light receiver the intensity of the light returning after passing through the living body (Configuration 5-8).

The sleep sensor of Configuration 5-8 can be so configured that the optical sensor has a box-shaped case; and a light-shielding wall which divides the case into a first region, where the light emitter is mounted, and a second region, where the light receiver is mounted (Configuration 5-9).

The sleep sensor of Configuration 5-9 can be so configured that, among the height H1 of the light-shielding wall, the height H2 of the light emitter, and the height H3 of the light receiver, the relationship H1>H2>H3 holds (Configuration 5-10).

The sleep sensor of Configuration 5-10 can be so configured that the case is buried in a housing which holds the optical sensor, in such a way that the case protrudes from the housing (Configuration 5-11).

The sleep sensor of Configuration 5-11 can be so configured that the optical sensor has, between itself and the housing, a damping member (Configuration 5-12).

The sleep sensor of any of Configurations 5-8 to 5-12 can be so configured that that the output wavelength of the light emitter is in a visible region of the spectrum, about 600 nm or less (Configuration 5-13).

Moreover, according to the fifth aspect of the present invention, a physical condition management system can have a sleep sensor of any one of Configurations 5-1 to 5-13 and an information terminal which analyzes and takes a log on the measurement data acquired by the sleep sensor (Configuration 5-14).

Furthermore, according to the fifth aspect of the present invention, a home appliance control system can have a sleep sensor of any one of Configurations 5-1 to 5-13 and a home electric appliance that is driven according to the test subject's sleep condition as determined by use of the sleep sensor or the input terminal (Configuration 5-15).

The home appliance control system of Configuration 5-15 can be so configured that the home electric appliance is at least one of an electrically-operated curtain, an audio appliance, a lighting appliance, a television, an air conditioner, and a bed appliance (Configuration 5-16).

[Sixth Aspect of the Invention]

Of the different aspects of the present invention disclosed herein, according to a sixth aspect, a pulse wave sensor can be configured as one having an optical sensor which irradiates a living body with light from a light emitter to detect with a light receiver the intensity of the light that has passed through the living body; a body which holds the optical sensor; a belt which is attached to the body and is wound around the living body; and a damping member which is provided between the optical sensor and the body (Configuration 6-1).

The pulse wave sensor of Configuration 6-1 can be so configured as to further have a printed circuit board on which the optical sensor is mounted, with the damping member arranged between the printed circuit board and the body (Configuration 6-2).

The pulse wave sensor of Configuration 6-1 or 6-2 can be so configured as to further have a close-contact member which is provided around the optical sensor to achieve close contact with the living body (Configuration 6-3).

The pulse wave sensor of Configuration 6-3 can be so configured that the close-contact member is arranged with a gap left from the optical sensor (Configuration 6-4).

The pulse wave sensor of any of Configurations 6-2 to 6-4 can be so configured as to further have a protective member which covers at least one of the obverse and reverse faces of the printed circuit board (Configuration 6-5).

The pulse wave sensor of Configuration 6-5 can be so configured that at least one of the close-contact member and the protective member is black in color (Configuration 6-6).

The pulse wave sensor of any of Configurations 6-2 to 6-6 can be so configured that the belt and the printed circuit board are attached to the body with such a gap left in between as to prevent mutual contact (Configuration 6-7).

The pulse wave sensor of any of Configurations 6-1 to 6-7 can be so configured that the body is given a low-center-of-gravity structure (Configuration 6-8).

The pulse wave sensor of any of Configurations 6-1 to 6-8 can be so configured as to have a filter which applies filtering to the output signal of the optical sensor (Configuration 6-9).

The pulse wave sensor of Configuration 6-9 can be so configured that the filter has a band-pass filter circuit which eliminates a low-frequency component and a high-frequency component from the output signal of the optical sensor (Configuration 6-10).

The pulse wave sensor of Configuration 6-10 can be so configured that the band-pass filter circuit is a sixth-order operational amplifier multiple-feedback band-path filter circuit having a pass band of 0.7 Hz to 3.0 Hz (Configuration 6-11).

According to the sixth aspect of the present invention, a pulse wave sensor can instead be configured as one having an optical sensor which irradiates a living body with light from a light emitter and detects with a light receiver the intensity of the light that has passed through the living body; a pulse driver which pulse-drives the light emitter with higher luminance than outside light; and a filter which applies detection to the output signal of the optical sensor to extract a pulse wave signal (Configuration 6-12).

The pulse wave sensor of Configuration 6-12 can be so configured that the wavelength characteristics of the light receiver match the wavelength characteristics of the light emitter (Configuration 6-13).

The pulse wave sensor of Configuration 6-12 or 6-13 can be so configured that the pulse driver pulse-drives the light emitter at a duty ratio of 1/10 to 1/100 (Configuration 6-14).

The pulse wave sensor of any of Configurations 6-12 to 6-14 can be so configured that the filter has a detector circuit which applies detection to the output signal of the optical sensor; a first amplifier circuit which amplifies the output signal of the detector circuit; a band-pass filter circuit which eliminates a low-frequency component and a high-frequency component from the output signal of the first amplifier circuit; a low-pass filter circuit which eliminates a high-frequency component from the output signal of the band-pass filter circuit; and a second amplifier circuit which amplifies the output signal of the low-pass filter circuit (Configuration 6-15).

The pulse wave sensor of Configuration 6-15 can be so configured that the band-pass filter circuit is a sixth-order operational amplifier multiple-feedback band-path filter circuit having a pass band of 0.7 Hz to 3.0 Hz (Configuration 6-16).

The pulse wave sensor of Configuration 6-15 or 6-16 can be so configured that the low-pass filter circuit is a first-order CR low-pass filter circuit having a cut-off frequency of 1.45 Hz (Configuration 6-17).

The pulse wave sensor of any of Configurations 6-15 to 6-17 can be so configured that the filter includes an intermediate voltage generator circuit which divides a supply voltage to produce an intermediate voltage, and the detector circuit, the first amplifier circuit, the band-pass filter circuit, the low-pass filter circuit, and the second amplifier circuit all operate relative to the intermediate voltage as a reference voltage (Configuration 6-18).

The pulse wave sensor of any of Configurations 6-1 to 6-18 can be so configured that the output wavelength of the light emitter is in a visible region of the spectrum, about 600 nm or less (Configuration 6-19).

[Seventh Aspect of the Invention]

Of the different aspects of the present invention disclosed herein, according to a seventh aspect, a pulse wave sensor can be configured as one having a housing which is worn on the outer ear; an optical sensor which acquires pulse wave data by irradiating the outer ear with light from a light emitter and detecting with a light receiver the intensity of the light returning after passing through the living body; and a damping member which is provided between the housing and the optical sensor (Configuration 7-1).

The pulse wave sensor of Configuration 7-1 can be so configured as to further have a close-contact member which enhances the ease of wearing on the outer ear (Configuration 7-2).

The pulse wave sensor of Configuration 7-2 can be so configured that the optical sensor is arranged at a position where the optical sensor is covered by the close-contact member, which transmits light (Configuration 7-3).

The pulse wave sensor of Configuration 7-3 can be so configured that the damping member is arranged between the housing and the optical sensor with the damping member compressed in its height direction (Configuration 7-4).

The pulse wave sensor of Configuration 7-4 can be so configured that the damping member is compressed by the contracting force of the close-contact member which covers the optical sensor (Configuration 7-5).

The pulse wave sensor of Configuration 7-4 or 7-5 can be so configured that the damping member is compressed by the binding force of leads laid from opposite ends of the optical sensor (Configuration 7-6).

The pulse wave sensor of any of Configurations 7-4 to 7-6 can be so configured that the damping member is compressed by the contracting force of an elastic member which couples the housing and the optical sensor together (Configuration 7-7).

The pulse wave sensor of any of Configurations 7-4 to 7-7 can be so configured that the damping member is compressed by the locking force of a protruding member which couples the housing and the optical sensor together (Configuration 7-8).

The pulse wave sensor of any of Configurations 7-4 to 7-8 can be so configured that the damping member, when uncompressed, has a height of 2.5±1.0 cm (Configuration 7-9).

The pulse wave sensor of any of Configurations 7-4 to 7-9 can be so configured as to further have a light-shielding member which prevents outside light from entering the optical sensor (Configuration 7-10).

The pulse wave sensor of Configuration 7-10 can be so configured that the close-contact member transmits light at the light emission wavelength only in a part of the close-contact member covering the optical sensor to serve as a measurement window, and elsewhere functions as the light-shielding member (Configuration 7-11).

The pulse wave sensor of any of Configurations 7-1 to 7-11 can be so configured that the damping member is formed of urethane sponge (Configuration 7-12).

The pulse wave sensor of any of Configurations 7-1 to 7-12 can be so configured that the light receiver is arranged closer to the external ear canal than the light emitter is (Configuration 7-13).

The pulse wave sensor of any of Configurations 7-1 to 7-13 can be so configured that the output wavelength of the light emitter is in a visible region of the spectrum, about 600 nm or less (Configuration 7-14).

According to the seventh aspect of the present invention, a pulse wave sensor can instead be configured as one having a housing which is worn on the outer ear; an optical sensor which is provided in the housing and which acquires pulse wave data by irradiating the outer ear with light from a light emitter and detecting with a light receiver the intensity of the light returning after passing through the living body; and a close-contact member which enhances the closeness of contact between the optical sensor and the outer ear (Configuration 7-15).

According to the seventh aspect of the present invention, a pulse wave sensor can instead be configured as one having a housing which is worn on the outer ear; an optical sensor provided in the housing and which acquires pulse wave data by irradiating the outer ear with light from a light emitter and detecting with a light receiver the intensity of the light returning after passing through the living body; and a light-shielding member which prevents outside light from entering the optical sensor (Configuration 7-16).

### Other Modified Examples

The different configurations according to the present invention disclosed herein, described by way of embodiments above, allow for various modifications without departing from the spirit of the invention. That is, the embodiments described above should be understood to be in every aspect merely illustrative and not restrictive; the technical scope of the present invention is defined not by the description of those specific embodiments but by the appended claims, and should be understood to encompass any modifications made in the sense and scope equivalent to those of the claims.

### INDUSTRIAL APPLICABILITY

The different aspects of the present invention disclosed herein can be exploited as a technology for enhancing the usability of pulse wave sensors and sleep sensors, and find applications in a variety of fields, such as health care support appliances, game appliances, music appliances, pet communication tools, and appliances for preventing vehicle drivers' drowsiness.

### LIST OF REFERENCE SIGNS

1 pulse wave sensor
2 living body (wrist, ear, etc.)
10 main unit
10a body
10b printed circuit board
10c damping member
10d close-contact member
10e protective member
11 optical sensor
11a case
11b light-shielding wall
11c condenser lens
11d, 11e lid member
11f damping member (rubber, synthetic sponge, etc.)
11z light-transmitting plate
11A light-emitting diode (light emitter)
11B phototransistor (light receiver)
12 filter
13 controller
14 display
15 communicator
16 power supply
17 pulse driver (modulator circuit)
20 belt
30 spring hinge
x light emitter (light-emitting chip)
y light receiver (light-receiving chip)
X light emitter
X1 substrate
X2 light-emitting chip
X3 seal
X4 wire
X5 conductor
X6 color filter
Y light receiver
Y1 substrate
Y2 light-receiving chip
Y3 seal
Y4 wire
Y5 conductor
Y6 color filter
100 current/voltage converter circuit
110 first-order CR high-pass filter circuit
120 amplifier circuit
130 first-order CR low-pass filter circuit
140 amplifier circuit
200 current/voltage converter circuit
210 first-order CR high-pass filter circuit
220 voltage follower circuit
230 second-order CR low-pass filter circuit
240 amplifier circuit
250 sixth-order band-pass filter circuit
260 amplifier circuit
270 intermediate voltage generator circuit
300 current/voltage converter circuit
310 detector circuit (demodulator circuit)
320 amplifier circuit
330 sixth-order band-pass filter circuit
340 first-order CR low-pass filter circuit
350 amplifier circuit
360 intermediate voltage generator circuit
R1-R55 resistor
C1-C43 capacitor
D1, D2 diode
OP1-0P14 operational amplifier
P1 P-channel MOS field-effect transistor
IC1 semiconductor device
ST1-ST3 Schmitt trigger
E outer ear
E1 scaphoid fossa
E2 helix
E3 antihelix
E4 antitragus
E5 external ear canal (external acoustic meatus)
E6 superior antihelical crus
E7 triangular fossa
E8 inferior antihelical crus
E9 concha auriculae
E10 tragus
E11 intertragic notch
E12 lobule
401 pulse wave sensor (portable audio player, hearing aid)
401X earphone (headphone)
401Y main unit
402 information terminal (data server, personal computer, etc.)
403 network
410 housing
410x protruding member
410y clip member
411 optical sensor
411A light emitter
411B light receiver
412 speaker
413 driver
414 cord
415 connector
420 housing
421 controller
422 operation panel
423 display
424 storage
425 communicator
426 power supply
427 filter
501 sleep sensor
501X eye mask-type housing
501Y sensor unit
501Z main unit
502 information terminal (data server, personal computer, etc.)
511 optical sensor
512 temperature sensor
513 acceleration sensor
514 microphone
515 controller
516 display
517 speaker
518 operation panel
519 storage
520 communicator
521 power supply
A1 electrically-operated curtain
A2 audio appliance
A3 lighting appliance
A4 television
A5 air conditioner
A6 bed appliance (electrically-operated bed, pneumatic mattress, etc.)
600 pulse wave sensor
610 housing
620 optical sensor
630 damping member
640 close-contact member (earpiece)
641 measurement window
650 lead
660 elastic member
670 protruding member
680 light-shielding member
690 close-contact member

1. A pulse wave sensor comprising:
a housing worn on an outer ear;
an optical sensor provided in the housing, the optical sensor acquiring pulse wave data by irradiating the outer ear with light from a light emitter and detecting, with a light receiver, intensity of light returning after passing through a living body; and
a damping member provided between the housing and the optical sensor.

2. The pulse wave sensor according to claim 1, further comprising a close-contact member configured to enhance ease of wearing on the outer ear.

3. The pulse wave sensor according to claim 2, wherein the optical sensor is arranged at a position where the optical sensor is covered by the close-contact member, which transmits light.

4. The pulse wave sensor according to claim 3, wherein the damping member is arranged between the housing and the optical sensor with the damping member compressed in a height direction thereof.

5. The pulse wave sensor according to claim 4, wherein the damping member is compressed by a contracting force of the close-contact member which covers the optical sensor.

6. The pulse wave sensor according to claim 4, wherein the damping member is compressed by a binding force of leads laid from opposite ends of the optical sensor.

7. The pulse wave sensor according to claim 4, wherein the damping member is compressed by a contracting force of an elastic member coupling the housing and the optical sensor together.

8. The pulse wave sensor according to claim 4, wherein the damping member is compressed by a locking force of a protruding member coupling the housing and the optical sensor together.

9. The pulse wave sensor according to claim 4, wherein the damping member, when uncompressed, has a height of 2.5±1.0 cm.

10. The pulse wave sensor according to claim 4, further comprising a light-shielding member preventing outside light from entering the optical sensor.

11. The pulse wave sensor according to claim 10, wherein the close-contact member transmits light at a light emission wavelength only in a part of the close-contact member covering the optical sensor to serve as a measurement window, and elsewhere functions as the light-shielding member.

12. The pulse wave sensor according to claim 1, wherein the damping member is formed of urethane sponge.

13. The pulse wave sensor according to claim 1, wherein the light receiver is arranged closer to an external ear canal than the light emitter is.

14. The pulse wave sensor according claim 1, wherein an output wavelength of the light emitter is in a visible region of a spectrum, about 600 nm or less.

15. A pulse wave sensor comprising:
a housing worn on an outer ear;
an optical sensor provided in the housing, the optical sensor acquiring pulse wave data by irradiating the outer ear with light from a light emitter and detecting, with a light receiver, intensity of light returning after passing through a living body; and
a close-contact member configured to enhance closeness of contact between the optical sensor and the outer ear.

16. A pulse wave sensor comprising:
a housing worn on an outer ear;
an optical sensor provided in the housing, the optical sensor acquiring pulse wave data by irradiating the outer ear with light from a light emitter and detecting, with a light receiver, intensity of light returning after passing through a living body; and
a light-shielding member preventing outside light from entering the optical sensor.
